(19) United States
(12) Patent Application Publication
(10) Pub. No.: US 2022/0396567 A1
(43) Pub. Date: Dec. 15, 2022

(54) SUBSTITUTED PYRIMIDINE FOR THE TREATMENT AND PROPHYLAXIS OF HEPATITIS B VIRUS INFECTION

(71) Applicant: Hoffmann-La Roche Inc., Little Falls, NJ (US)

(72) Inventors: Song FENG, Shanghai (CN); Xingchun HAN, Shanghai (CN); Chungen LIANG, Shanghai (CN); Kun MIAO, Shanghai (CN); Hong SHEN, Shanghai (CN); Xuefei TAN, Shanghai (CN); Jianping WANG, Shanghai (CN); Li WANG, Shanghai (CN)

(73) Assignee: Hoffmann-La Roche Inc., Little Falls, NJ (US)

(21) Appl. No.: 17/764,933

(22) PCT Filed: Sep. 28, 2020
(86) PCT No.: PCT/EP2020/077028

§ 371 Date: Mar. 29, 2022

(30) Foreign Application Priority Data
Sep. 30, 2019 (CN) PCT/CN2019/109509

(51) Int. Cl. C07D 405/04; C07D 405/14; C07D 409/04

(52) U.S. Cl. CPC C07D 405/04; C07D 405/14; C07D 409/04

(57) ABSTRACT

The present invention provides novel compounds having the general formula: wherein ^{R1} to R⁴, L₁, L₂ and X are as described herein, compositions including the compounds and methods of using the compounds.

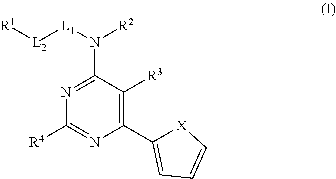

The present invention relates to organic compounds useful for therapy and/or prophylaxis of HBV infection in a mammal, and in particular to cccDNA (covalently closed circular DNA) inhibitors useful for treating HBV infection.

### FIELD OF THE INVENTION

The present invention relates to substituted pyrimidine having pharmaceutical activity, their manufacture, pharmaceutical compositions containing them and their potential use as medicaments.

The present invention relates to compounds of formula (I)

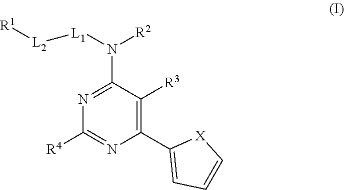

wherein R¹ to R⁴, L₁, L₂ and X are as described below, or a pharmaceutically acceptable salt thereof.

Hepatitis B virus (HBV) infection is one of the most prevalent viral infections and is a leading cause of chronic hepatitis. It is estimated that worldwide, around 2 billion people have evidence of past or present infection with HBV. Over 250 million individuals are currently chronically infected with HBV and are therefore at high risk to develop liver fibrosis, cirrhosis and hepatocellular carcinoma (HCC). There are data to indicate ˜800,000 deaths per year are directly linked to HBV infection (Lozano, R. et al., Lancet (2012), 380 (9859), 2095-2128; Goldstein, S. T. et al., Int J Epidemiol (2005), 34 (6), 1329-1339).

Many countries in the world administer hepatitis B vaccine starting at birth or in early childhood, which has greatly reduced the incidence and prevalence of hepatitis B in most endemic regions over the past few decades. However, the vaccine has no impact on people who were infected before the widely use of the vaccine in developing end-stage liver disease or HCC (Chen, D. S., J Hepatol (2009), 50 (4), 805-816). Vaccination at birth of infants born to HBV positive mothers is usually not sufficient for protecting vertical transmission and combination with hepatitis B immune globulin is needed (L₁, X. M. et al., World J Gastroenterol (2003), 9 (7), 1501-1503).

Currently FDA-approved treatments for chronic hepatitis B include two type 1 interferons (IFN) which are IFNalfa-2b and pegylated IFN alfa-2a and six nucleos(t)ide analogues (NAs) which are lamivudine (3TC), tenofovir disoproxil fumarate (TDF), adefovir (ADV), telbivudine (LdT), entecavir (ETV), and vemlidy (tenofovir alafenamide (TAF)). IFN treatment is finite, but it is known to have severe side effects, and only a small percentage of patients showed a sustained virological response, measured as loss of hepatitis B surface antigen (HBsAg). NAs are inhibitors of the HBV reverse transcriptase, profoundly reduce the viral load in vast majority of treated patients, and lead to improvement of liver function and reduced incidence of liver failure and hepatocellular carcinoma. However, the treatment of NAs is infinite (Ahmed, M. et al., Drug Discov Today (2015), 20 (5), 548-561; Zoulim, F. and Locarnini, S., Gastroenterology (2009), (5), 1593-1608 e1591-1592).

HBV chronic infection is caused by persistence of covalently closed circular (ccc) DNA, which exists as an episomal form in hepatocyte nuclei. cccDNA serves as the template for viral RNA transcription and subsequent viral DNA generation. Only a few copies of cccDNA per liver cell can establish or re-initiate viral replication. Therefore, a complete cure of chronic hepatitis B will require elimination of cccDNA or permanently silencing of cccDNA. However, cccDNA is intrinsically very stable and currently available therapeutics could not eliminate cccDNA or permanently silence cccDNA (Nassal, M., Gut (2015), 64 (12), 1972-1984; Gish, R. G. et al., Antiviral Res (2015), 121, 47-58; Levrero, M. et al., J Hepatol (2009), 51 (3), 581-592). The current SoC could not eliminate the cccDNA which are already present in the infected cells. There is an urgent need to discover and develop new anti-HBV reagents to eliminate or permanently silence cccDNA, the source of chronicity (Ahmed, M. et al., Drug Discov Today (2015), 20 (5), 548-561; Nassal, M., Gut (2015), 64 (12), 1972-1984).

### SUMMARY OF THE INVENTION

Objects of the present invention are compounds of formula (I), their manufacture, medicaments based on a compound in accordance with the invention and their production as well as the use of compounds of formula (I) as cccDNA inhibitors and for the treatment or prophylaxis of HBV infection. The compounds of formula (I) show superior anti-HBV activity. In addition, the compounds of formula (I) also show good PK profiles.

The present invention relates to a compound of formula (I)

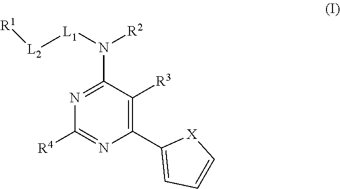

wherein
R¹ is C₁₋₆ alkyl, C₃₋₇ cycloalkyl, phenyl or 4-6 membered heterocyclyl; wherein C₁₋₆ alkyl, C₃₋₇ cycloalkyl, phenyl and 4-6 membered heterocyclyl are unsubstituted or substituted by one or two or three or four substituents independently selected from C₁₋₆ alkyl, C₁₋₆ alkoxy, halogen, haloC₁₋₆ alkyl, C₁₋₆ alkylsulfonyl and amino;
R² is H, halogen, C₁₋₆ alkyl or haloC₁₋₆ alkyl;
R³ is H, halogen, C₁₋₆ alkyl, haloC₁₋₆ alkyl, CN, C₁₋₆ alkoxy, carboxy, C₁₋₆ alkoxycarbonyl, (4-6 membered heterocyclyl)carbonyl or C₁₋₆ alkoxycarbonyl(4-6 membered heterocyclyl)carbonyl;
R⁴ is H, halogen, C₁₋₆ alkyl, haloC₁₋₆ alkyl, C₁₋₆ alkoxy, amino, C₁₋₆ alkylamino, (C₁₋₆ alkyl)₂ amino, haloC₁₋₆ alkylamino, C₁₋₆ alkoxyC₁₋₆ alkylamino, C₁₋₆ alkylsulfanyl or 2-azaspiro[3.3]heptanyl;
L₁ is a bond, —CH₂—, —CH₂CH₂—, —CH(CH₃)—, —CH₂CH₂CH₂— or —CH₂CH (halogen)CH₂—;
L₂ is a bond, 4-6 membered heterocyclyl or C₃₋₇ cycloalkyl;
X is NH, O or S;
or a pharmaceutically acceptable salt thereof.

### DETAILED DESCRIPTION OF THE INVENTION

### Definitions

As used herein, the term “C₁₋₆ alkyl” alone or in combination signifies a saturated, linear- or branched chain alkyl group containing 1 to 6, particularly 1 to 4 carbon atoms, for example methyl, ethyl, propyl, isopropyl, butyl, isobutyl, tert-butyl and the like. Particular “C₁₋₆ alkyl” groups are methyl, ethyl, propyl, isopropyl, isobutyl and tert-butyl. Most particular “C₁₋₆ alkyl” group is methyl and ethyl.

The term “C₁₋₆ alkoxy” alone or in combination signifies a group C₁₋₆ alkyl-O—, wherein the “C₁-6 alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆ alkoxy” groups are methoxy, ethoxy or butoxy.

The term “C₃₋₇ cycloalkyl” denotes to a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇ cycloalkyl” groups are cyclopropyl, cyclobutyl, cyclopentyl or cyclohexyl.

The term “halogen” and “halo” are used interchangeably herein and denote fluoro, chloro, bromo, or iodo.

The term “haloC₁₋₆ alkyl” denotes an alkyl group wherein at least one of the hydrogen atoms of the alkyl group is replaced by same or different halogen atoms, particularly fluoro atoms. Examples of haloC₁₋₆ alkyl include monochloro-, difluoro- or trifluoro-methyl, -ethyl or -propyl, for example difluoromethyl and trifluoromethyl.

The term “carbonyl” alone or in combination refers to the group —C(O)—.

The term “sulfonyl” alone or in combination refers to the group —S(O)₂—.

The term “sulfanyl” alone or in combination refers to the group —S—.

“Heterocyclyl” refers to any mono-, bi-, tricyclic or spiro, saturated or unsaturated, aromatic (heteroaryl) or non-aromatic (e.g., heterocycloalkyl), ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocyclyl, regardless of the point of attachment of the cyclic system to the rest of the molecule. In one example, heterocyclyl includes 3-11 ring atoms (“members”) and includes monocycles, bicycles, tricycles and spiro ring systems, wherein the ring atoms are carbon, where at least one atom in the ring or ring system is a heteroatom selected from nitrogen, sulfur or oxygen. In one example, heterocyclyl includes 3- to 7-membered monocycles having 1, 2, 3 or 4 heteroatoms selected from nitrogen, sulfur or oxygen. In another example, heterocyclyl includes 4-, 5- or 6-membered monocycles having 1, 2, 3 or 4 heteroatoms selected from nitrogen, sulfur or oxygen. Examplary heterocyclyls are azetidinyl, pyrrolidinyl, piperidyl, morpholino, piperazinyl, tetrahydropyranyl and 2-azaspiro[3.3]heptanyl. Heterocyclyl may be optionally substituted by halogen, OH, SH, cyano, NH₂, NHCH₃, N(CH₃)₂, NO₂, N₃, C(O)CH₃, COOH, CO₂CH₃, C₁₋₆ alkyl, C₁₋₆ alkoxy, oxo, haloC₁₋₆ alkyl, hydroxyC₁₋₆ alkoxy, C₁₋₆ alkylsulfonyl, C₁₋₆ alkoxycarbonylphenyl, carboxyC₁₋₆ alkoxyC₁₋₆ alkoxy, carboxyC₃₋₇ cycloalkylC₁₋₆ alkoxy, phenyl or heterocyclyl.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

### HBV Inhibitors

The present invention provides (i) a compound having the general formula (I):

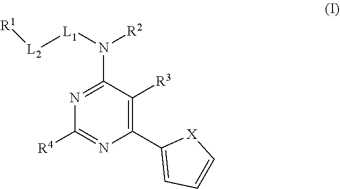

wherein
R¹ is C₁₋₆ alkyl, C₃₋₇ cycloalkyl, phenyl or 4-6 membered heterocyclyl; wherein C₁₋₆ alkyl, C₃₋₇ cycloalkyl, phenyl and 4-6 membered heterocyclyl are unsubstituted or substituted by one or two or three or four substituents independently selected from C₁₋₆ alkyl, C₁₋₆ alkoxy, halogen, haloC₁₋₆ alkyl, C₁₋₆ alkylsulfonyl and amino;
R² is H, halogen, C₁₋₆ alkyl or haloC₁₋₆ alkyl;
R³ is H, halogen, C₁₋₆ alkyl, haloC₁₋₆ alkyl, CN, C₁₋₆ alkoxy, carboxy, C₁₋₆ alkoxycarbonyl, (4-6 membered heterocyclyl)carbonyl or C₁₋₆ alkoxycarbonyl(4-6 membered heterocyclyl)carbonyl;
R⁴ is H, halogen, C₁₋₆ alkyl, haloC₁₋₆ alkyl, C₁₋₆ alkoxy, amino, C₁₋₆ alkylamino, (C₁₋₆ alkyl)₂ amino, haloC₁₋₆ alkylamino, C₁₋₆ alkoxyC₁₋₆ alkylamino, C₁₋₆ alkylsulfanyl or 2-azaspiro[3.3]heptanyl;
L₁ is a bond, —CH₂—, —CH₂CH₂—, —CH(CH₃)—, —CH₂CH₂CH₂— or —CH₂CH (halogen)CH₂—;
L₂ is a bond, 4-6 membered heterocyclyl or C₃₋₇ cycloalkyl;
X is NH, O or S;
or a pharmaceutically acceptable salt thereof.

A further embodiment of the present invention is (ii) a compound of formula (I) according to (i), or a pharmaceutically acceptable salt thereof, wherein R¹ is C₁₋₆ alkyl, C₃₋₇ cycloalkyl, phenyl or tetrahydropyranyl; wherein C₃₋₇ cycloalkyl, phenyl and tetrahydropyranyl are unsubstituted or substituted by one or two or three or four substituents independently selected from C₁₋₆ alkyl, C₁₋₆ alkoxy, halogen, haloC₁₋₆ alkyl, C₁₋₆ alkylsulfonyl and amino.

A further embodiment of the present invention is (iii) a compound of formula (I) according to (i) or (ii), or a pharmaceutically acceptable salt thereof, wherein R¹ is C₃₋₇ cycloalkyl, phenyl or tetrahydropyranyl; wherein phenyl and tetrahydropyranyl are unsubstituted or substituted by one or two or three or four substituents independently selected from C₁₋₆ alkyl, halogen, haloC₁₋₆ alkyl, C₁₋₆ alkylsulfonyl and amino.

A further embodiment of the present invention is (iv) a compound of formula (I) according to any one of (i) to (iii), or a pharmaceutically acceptable salt thereof, wherein R¹ is cyclopentyl, phenyl or tetrahydropyranyl; wherein phenyl and tetrahydropyranyl are unsubstituted or substituted by one or two or three or four substituents independently selected from methyl, Cl, CF₃, methylsulfonyl and amino.

A further embodiment of the present invention is (v) a compound of formula (I) according to any one of (i) to (iv), or a pharmaceutically acceptable salt thereof, wherein R² is H or C₁₋₆ alkyl.

A further embodiment of the present invention is (vi) a compound of formula (I) according to any one of (i) to (v), or a pharmaceutically acceptable salt thereof, wherein R² is H.

A further embodiment of the present invention is (vii) a compound of formula (I) according to any one of (i) to (vi), or a pharmaceutically acceptable salt thereof, wherein R³ is H, CN, C₁₋₆ alkoxy, carboxy, C₁₋₆ alkoxycarbonyl, azetidinylcarbonyl, pyrrolidinylcarbonyl, piperidylcarbonyl, morpholinocarbonyl or C₁₋₆ alkoxycarbonylpiperazinylcarbonyl.

A further embodiment of the present invention is (viii) a compound of formula (I) according to any one of (i) to (vii), or a pharmaceutically acceptable salt thereof, wherein R³ is CN or carboxy.

A further embodiment of the present invention is (ix) a compound of formula (I) according to any one of (i) to (viii), or a pharmaceutically acceptable salt thereof, wherein R⁴ is H, C₁₋₆ alkyl, C₁-6 alkoxy, amino, C₁-6 alkylamino, (C₁-6 alkyl)₂ amino, haloC₁₋₆ alkylamino, C₁-6 alkoxyC₁₋₆ alkylamino, C₁-6 alkylsulfanyl or 2-azaspiro[3.3]heptanyl.

A further embodiment of the present invention is (x) a compound of formula (I) according to any one of (i) to (ix), or a pharmaceutically acceptable salt thereof, wherein R⁴ is H, C₁₋₆ alkyl, C₁₋₆ alkoxy, amino, C₁₋₆ alkylamino or C₁₋₆ alkoxyC₁₋₆ alkylamino.

A further embodiment of the present invention is (xi) a compound of formula (I) according to any one of (i) to (x), or a pharmaceutically acceptable salt thereof, wherein R⁴ is H, methyl, butoxy, amino, ethylamino or methoxyethylamino.

A further embodiment of the present invention is (xii) a compound of formula (I) according to any one of (i) to (xi), or a pharmaceutically acceptable salt thereof, wherein L₁ is a bond, —CH₂—, —CH₂CH₂—, —CH(CH₃)— or —CH₂CH(F)CH₂—.

A further embodiment of the present invention is (xiii) a compound of formula (I) according to any one of (i) to (xii), or a pharmaceutically acceptable salt thereof, wherein L₁ is a bond, —CH₂— or —CH(CH₃)—.

A further embodiment of the present invention is (xiv) a compound of formula (I) according to any one of (i) to (xiii), or a pharmaceutically acceptable salt thereof, wherein L₂ is a bond or C₃₋₇ cycloalkyl.

A further embodiment of the present invention is (xv) a compound of formula (I) according to any one of (i) to (xiv), or a pharmaceutically acceptable salt thereof, wherein L₂ is a bond or cyclopropyl.

A further embodiment of the present invention is (xvi) a compound of formula (I) according to any one of (i) to (xv), or a pharmaceutically acceptable salt thereof, wherein X is O or S.

A further embodiment of the present invention is (xvii) a compound of formula (I) according to any one of (i) to (xvi), or a pharmaceutically acceptable salt thereof, wherein X is O.

A further embodiment of the present invention is (xviii) a compound of formula (I) according to (i), wherein
R¹ is C₁₋₆ alkyl, C₃₋₇ cycloalkyl, phenyl or tetrahydropyranyl; wherein C₃₋₇ cycloalkyl, phenyl and tetrahydropyranyl are unsubstituted or substituted by one or two or three or four substituents independently selected from C₁₋₆ alkyl, C₁₋₆ alkoxy, halogen, haloC₁₋₆ alkyl, C₁₋₆ alkylsulfonyl and amino;
R² is H or C₁₋₆ alkyl;
R³ is H, CN, C₁₋₆ alkoxy, carboxy, C₁₋₆ alkoxycarbonyl, azetidinylcarbonyl, pyrrolidinylcarbonyl, piperidylcarbonyl, morpholinocarbonyl or C₁₋₆ alkoxycarbonylpiperazinylcarbonyl;
R⁴ is H, C₁₋₆ alkyl, C₁₋₆ alkoxy, amino, C₁₋₆ alkylamino, (C₁₋₆ alkyl)₂ amino, haloC₁₋₆ alkylamino, C₁₋₆ alkoxyC₁₋₆ alkylamino, C₁₋₆ alkylsulfanyl or 2-azaspiro[3.3]heptanyl;
L₁ is a bond, —CH₂—, —CH₂CH₂—, —CH(CH₃)— or —CH₂CH(F)CH₂—;
L₂ is a bond or C₃₋₇ cycloalkyl;
X is O or S;
or a pharmaceutically acceptable salt thereof.

A further embodiment of the present invention is (xix) a compound of formula (I) according to (i), wherein
R¹ is C₃₋₇ cycloalkyl, phenyl or tetrahydropyranyl; wherein phenyl and tetrahydropyranyl are unsubstituted or substituted by one or two or three or four substituents independently selected from C₁₋₆ alkyl, halogen, haloC₁₋₆ alkyl, C₁₋₆ alkylsulfonyl and amino;
R² is H;
R³ is CN or carboxy;
R⁴ is H, C₁₋₆ alkyl, C₁₋₆ alkoxy, amino, C₁₋₆ alkylamino or C₁₋₆ alkoxyC₁₋₆ alkylamino;
L₁ is a bond, —CH₂— or —CH(CH₃)—;
L₂ is a bond or C₃₋₇ cycloalkyl;
X is O;
or a pharmaceutically acceptable salt thereof.

A further embodiment of the present invention is (xx) a compound of formula (I) according to (i), wherein
R¹ is cyclopentyl, phenyl or tetrahydropyranyl; wherein phenyl and tetrahydropyranyl are unsubstituted or substituted by one or two or three or four substituents independently selected from methyl, Cl, CF₃, methylsulfonyl and amino;
R² is H;
R³ is CN or carboxy;
R⁴ is H, methyl, butoxy, amino, ethylamino or methoxyethylamino;
L₁ is a bond, —CH₂— or —CH(CH₃)—;
L₂ is a bond or cyclopropyl;
X is O;
or a pharmaceutically acceptable salt thereof.

In another embodiment (xxi) of the present invention, particular compounds of the present invention are selected from:
2-amino-4-(2-furyl)-6-(1-phenylethylamino)pyrimidine-5-carbonitrile;
2-amino-4-[(3-bromo-4-isopropoxy-phenyl)methylamino]-6-(2-furyl)pyrimidine-5-carbonitrile;
2-amino-4-(2-furyl)-6-[(2,2,6,6-tetramethyltetrahydropyran-4-yl)methylamino]pyrimidine-5-carbonitrile;
2-amino-4-[[3-(2-chlorophenyl)-2-fluoro-propyl]amino]-6-(2-furyl)pyrimidine-5-carbonitrile;
2-amino-4-(2-furyl)-6-[1-[3-(trifluoromethyl)phenyl]ethylamino]pyrimidine-5-carbonitrile;
2-amino-4-(2-furyl)-6-[(3-methoxyphenyl)methyl-methyl-amino]pyrimidine-5-carbonitrile;
2-amino-4-(2-furyl)-6-[[1-(2-methylsulfonylphenyl)cyclopropyl]amino]pyrimidine-5-carbonitrile;
2-amino-4-[(3,3-difluorocyclopentyl)methylamino]-6-(2-furyl)pyrimidine-5-carbonitrile;
2-amino-4-(cyclopentylamino)-6-(2-furyl)pyrimidine-5-carbonitrile;
2-amino-4-(cyclohexylmethylamino)-6-(2-furyl)pyrimidine-5-carbonitrile;
4-(2-furyl)-2-methylsulfanyl-6-[[3-(trifluoromethyl)phenyl]methylamino]pyrimidine-5-carbonitrile;
4-(2-furyl)-2-(2-methoxyethylamino)-6-[[3-(trifluoromethyl)phenyl]methylamino]pyrimidine-5-carbonitrile;
4-(2-furyl)-2-(2,2,2-trifluoroethylamino)-6-[[3-(trifluoromethyl)phenyl]methylamino]pyrimidine-5-carbonitrile;
2-(ethylamino)-4-(2-furyl)-6-[[3-(trifluoromethyl)phenyl]methylamino]pyrimidine-5-carbonitrile;
4-(2-furyl)-2-(propylamino)-6-[[3-(trifluoromethyl)phenyl]methylamino]pyrimidine-5-carbonitrile;
2-(dimethylamino)-4-(2-furyl)-6-[[3-(trifluoromethyl)phenyl]methylamino]pyrimidine-5-carbonitrile;
4-(2-furyl)-2-methoxy-6-[[3-(trifluoromethyl)phenyl]methylamino]pyrimidine-5-carbonitrile;
2-ethoxy-4-(2-furyl)-6-[[3-(trifluoromethyl)phenyl]methylamino]pyrimidine-5-carbonitrile;
2-butoxy-4-(2-furyl)-6-[[3-(trifluoromethyl)phenyl]methylamino]pyrimidine-5-carbonitrile;
4-[(6-bromo-2,3-dimethoxy-phenyl)methylamino]-6-(2-furyl)-2-methyl-pyrimidine-5-carbonitrile;
4-[(3-bromo-4-isopropoxy-phenyl)methylamino]-6-(2-furyl)-2-methyl-pyrimidine-5-carbonitrile;
4-(2-furyl)-2-methyl-6-[[1-(2-methylsulfonylphenyl)cyclopropyl]amino]pyrimidine-5-carbonitrile;
4-(benzylamino)-6-(2-furyl)-2-methyl-pyrimidine-5-carbonitrile;
4-[(2-chlorophenyl)methylamino]-6-(2-furyl)pyrimidine-5-carbonitrile;
4-(benzylamino)-6-(2-furyl)pyrimidine-5-carbonitrile;
4-(2-furyl)-6-(isobutylamino)pyrimidine-5-carbonitrile;
4-(cyclohexylmethylamino)-6-(2-furyl)pyrimidine-5-carbonitrile;
4-(2-furyl)-6-(2-phenylethylamino)pyrimidine-5-carbonitrile;
4-(cyclopentylmethylamino)-6-(2-furyl)pyrimidine-5-carbonitrile;
4-(2-furyl)-6-[(1-phenylcyclopentyl)methylamino]pyrimidine-5-carbonitrile;
4-[(2-aminophenyl)methylamino]-6-(2-furyl)pyrimidine-5-carbonitrile;
6-(2-furyl)-N2,N2-dimethyl-N4-[1-[3-(trifluoromethyl)phenyl]ethyl]pyrimidine-2,4-diamine;
N4-[(2-aminophenyl)methyl]-6-(2-furyl)-N2,N2-dimethyl-pyrimidine-2,4-diamine;
6-(2-furyl)-2-methoxy-N-[1-[3-(trifluoromethyl)phenyl]ethyl]pyrimidin-4-amine;
6-(2-furyl)-5-methoxy-N4-[[3-(trifluoromethyl)phenyl]methyl]pyrimidine-2,4-diamine;
N4-[(2-aminophenyl)methyl]-6-(2-thienyl)pyrimidine-2,4-diamine;
2-amino-4-[(4-chlorophenyl)methylamino]-6-(2-furyl)pyrimidine-5-carboxylic acid;
2-amino-4-(2-furyl)-6-[[3-(trifluoromethyl)phenyl]methylamino]pyrimidine-5-carboxylic acid;
2-amino-4-(2-furyl)-6-(o-tolylmethylamino)pyrimidine-5-carboxylic acid;
2-amino-4-(2-furyl)-6-[(2-methoxyphenyl)methylamino]pyrimidine-5-carboxylic acid;
2-amino-4-(cyclopentylmethylamino)-6-(2-furyl)pyrimidine-5-carboxylic acid;
2-amino-4-[benzyl(methyl)amino]-6-(2-furyl)pyrimidine-5-carboxylic acid;
4-(cyclopentylmethylamino)-2-(dimethylamino)-6-(2-furyl)pyrimidine-5-carboxylic acid;
ethyl 2-amino-4-[(6-bromo-2,3-dimethoxy-phenyl)methylamino]-6-(2-furyl)pyrimidine-5-carboxylate;
ethyl 4-(2-furyl)-2-methylsulfanyl-6-[[3-(trifluoromethyl)phenyl]methylamino]pyrimidine-5-carboxylate;
4-(2-furyl)-2-(2-methoxyethylamino)-6-[[3-(trifluoromethyl)phenyl]methylamino]pyrimidine-5-carboxylic acid;
ethyl 2-(2-azaspiro[3.3]heptan-2-yl)-4-(2-furyl)-6-[[3-(trifluoromethyl)phenyl]methylamino]pyrimidine-5-carboxylate;
2-(2-azaspiro[3.3]heptan-2-yl)-4-(2-furyl)-6-[[3-(trifluoromethyl)phenyl]methylamino]pyrimidine-5-carboxylic acid;
ethyl 4-(2-furyl)-2-(isobutylamino)-6-[[3-(trifluoromethyl)phenyl]methylamino]pyrimidine-5-carboxylate;
4-(2-furyl)-2-(isobutylamino)-6-[[3-(trifluoromethyl)phenyl]methylamino]pyrimidine-5-carboxylic acid;
4-(2-furyl)-2-(3-methoxypropylamino)-6-[[3-(trifluoromethyl)phenyl]methylamino]pyrimidine-5-carboxylic acid;
[2-amino-4-(2-furyl)-6-[[3-(trifluoromethyl)phenyl]methylamino]pyrimidin-5-yl]-(azetidin-1-yl)methanone;
[2-amino-4-(2-furyl)-6-[[3-(trifluoromethyl)phenyl]methylamino]pyrimidin-5-yl]-pyrrolidin-1-yl-methanone;
[2-amino-4-(2-furyl)-6-[[3-(trifluoromethyl)phenyl]methylamino]pyrimidin-5-yl]-(1-piperidyl)methanone;
[2-amino-4-(2-furyl)-6-[[3-(trifluoromethyl)phenyl]methylamino]pyrimidin-5-yl]-morpholino-methanone;
tert-butyl 4-[2-amino-4-(2-furyl)-6-[[3-(trifluoromethyl)phenyl]methylamino]pyrimidine-5-carbonyl]piperazine-1-carboxylate;
ethyl 4-[(2-chlorophenyl)methylamino]-6-(2-furyl)pyrimidine-5-carboxylate;
4-(2-furyl)-2-(2-methoxyethylamino)-6-[[1-[3-(trifluoromethyl)phenyl]cyclopropyl]amino]pyrimidine-5-carboxylic acid;
4-[(3-chlorophenyl)methylamino]-6-(2-furyl)-2-(2-methoxyethylamino)pyrimidine-5-carboxylic acid;
2-(ethylamino)-4-(2-furyl)-6-[[1-[3-(trifluoromethyl)phenyl]cyclopropyl]amino]pyrimidine-5-carboxylic acid;
ethyl 4-(2-furyl)-2-methyl-6-[[3-(trifluoromethyl)phenyl]methylamino]pyrimidine-5-carboxylate;
and ethyl 4-[(6-bromo-2,3-dimethoxy-phenyl)methylamino]-6-(2-furyl)-2-isopropyl-pyrimidine-5-carboxylate;
or a pharmaceutically acceptable salt thereof.

In another embodiment (xxii) of the present invention, particular compounds of the present invention are selected from:
2-amino-4-(2-furyl)-6-[(2,2,6,6-tetramethyltetrahydropyran-4-yl)methylamino]pyrimidine-5-carbonitrile;
2-amino-4-(2-furyl)-6-[1-[3-(trifluoromethyl)phenyl]ethylamino]pyrimidine-5-carbonitrile;
2-amino-4-(2-furyl)-6-[[1-(2-methylsulfonylphenyl)cyclopropyl]amino]pyrimidine-5-carbonitrile;
2-(ethylamino)-4-(2-furyl)-6-[[3-(trifluoromethyl)phenyl]methylamino]pyrimidine-5-carbonitrile;
2-butoxy-4-(2-furyl)-6-[[3-(trifluoromethyl)phenyl]methylamino]pyrimidine-5-carbonitrile;
4-(2-furyl)-2-methyl-6-[[1-(2-methylsulfonylphenyl)cyclopropyl]amino]pyrimidine-5-carbonitrile;
4-[(2-chlorophenyl)methylamino]-6-(2-furyl)pyrimidine-5-carbonitrile;
4-(benzylamino)-6-(2-furyl)pyrimidine-5-carbonitrile;
4-(cyclopentylmethylamino)-6-(2-furyl)pyrimidine-5-carbonitrile;
4-[(2-aminophenyl)methylamino]-6-(2-furyl)pyrimidine-5-carbonitrile;
2-amino-4-[(4-chlorophenyl)methylamino]-6-(2-furyl)pyrimidine-5-carboxylic acid;
2-amino-4-(2-furyl)-6-[[3-(trifluoromethyl)phenyl]methylamino]pyrimidine-5-carboxylic acid; and
4-[(3-chlorophenyl)methylamino]-6-(2-furyl)-2-(2-methoxyethylamino)pyrimidine-5-carboxylic acid;
   or a pharmaceutically acceptable salt thereof.

### SYNTHESIS

The compounds of the present invention can be prepared by any conventional means. Suitable processes for synthesizing these compounds as well as their starting materials are provided in the schemes below and in the examples. All substituents, in particular, R¹ to R⁷, L₁, L₂ and X are as defined above unless otherwise indicated. Furthermore, and unless explicitly otherwise stated, all reactions, reaction conditions, abbreviations and symbols have the meanings well known to a person of ordinary skill in organic chemistry.

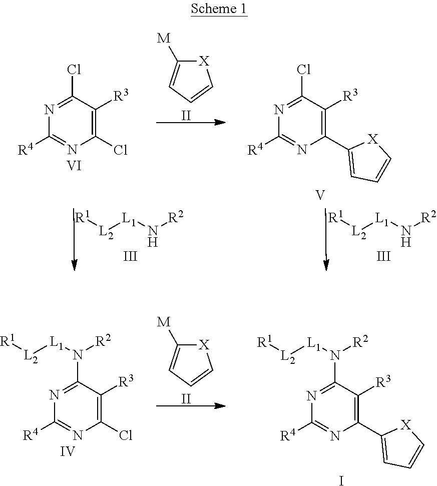

Wherein M is B(OH)₂ or SnBu₃.

Cross coupling reaction of a compound of formula VI with a compound of formula II in the presence of Pd catalyst, in a suitable solvent, such as DMF or 1,4-dioxane, at a suitable temperature, 100° C.-130° C., to give a compound of formula V. Substitution of the compound of formula V with a compound of formula III under room temperature, heat or microwave condition, in a suitable solvent, such as DMF, CH₃CN, DMSO or DCM, in the presence of a base, such as triethylamine, DIPEA or K₂CO₃, affords a compound of formula I.

The compound of formula I also can be prepared by reaction of the compound of formula VI with the compound of formula III at room temperature, in a suitable solvent, such as DCM or CH₃CN, followed by cross coupling reaction with the compound of formula II in the presence of Pd catalyst, in a suitable solvent, such as DMF or 1,4-dioxane at 90° C.-120° C.

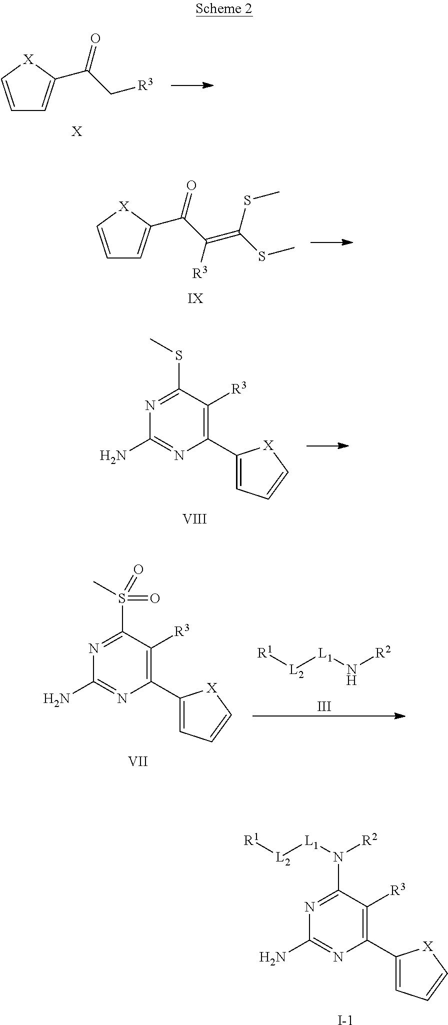

A compound of formula X reacts with CS₂ in the presence of NaH, followed by methylation in the presence of Mel, to give a compound of formula IX. The compound of formula IX reacts with guanidine carbonate in the presence of triethylamine, to give a compound of formula VIII. Oxidation of the compound of formula VIII in the presence of an oxidate, such as m-CPBA, in a suitable solvent, such as DCM, affords a compound of formula VII. Substitution of the compound of formula VII with a compound of formula III, in the presence of a suitable base, such as K₂CO₃, DIPEA or triethyl amine, in a suitable solvent, such as DMF or DMSO under heat or microwave condition, affords a compound of formula I-1.

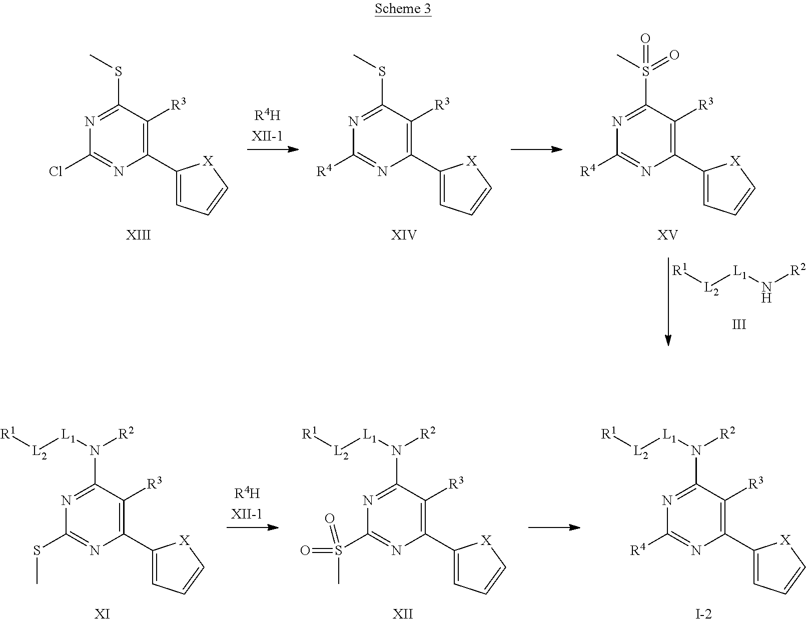

Oxidation of a compound of formula XI in the presence of an oxidate, such as m-CPBA, in a suitable solvent, such as DCM, affords a compound of formula XII. Substitution of the compound of formula XII with a compound of formula XII-1 under heat or microwave, in a suitable solvent, such as DMF or CH₃CN, gives a compound of formula I-2.

The compound of formula I-2 also can be prepared by the following steps. A compound of formula XIV can be prepared by a reaction of a compound of formula XIII and a compound of formula XII-1 under room temperature, heat or microwave condition, in a suitable solvent, such as DMF, CH₃CN or DCM. Oxidation of the compound of formula XIV in the presence of an oxidate, such as m-CPBA, in a suitable solvent, such as DCM, affords a compound of formula XV. Substitution of the compound of formula XV with the compound of formula III, in the presence of a suitable base, such as K₂CO₃, DIPEA or triethyl amine, in a suitable solvent, such as DMF or DMSO under heat or microwave condition, affords the compound of formula I-2.

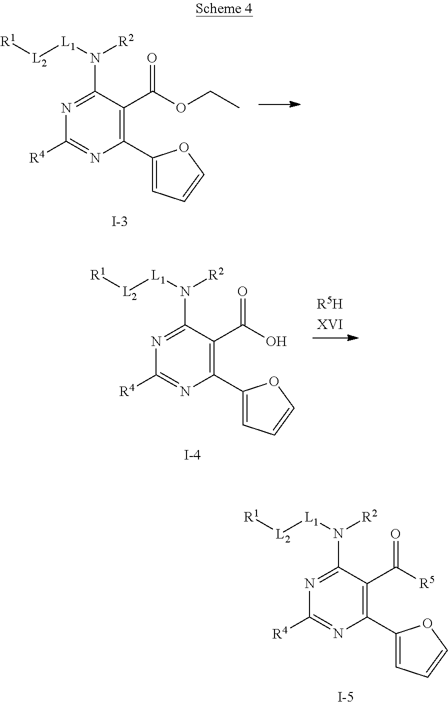

Wherein R⁵ is C₁₋₆ alkoxy, 4-6 membered heterocyclyl or C₁₋₆ alkoxycarbonyl(4-6 membered heterocyclyl); Hydrolysis of a compound of formula I-3 in the presence of a suitable base, such as KOH, LiOH or NaOH, in a suitable mixed solvent, such as THF/H₂O, CH₃CN/H₂O, DMF/H₂O or 1,4-dioxane/H₂O, affords a compound of formula I-4. Coupling of the compound of formula I-4 with a compound of formula XVI in the presence of a coupling reagent, such as EDCI, HATU or T₃P, and a base, such as DMAP, TEA or DIPEA, in a solvent, such as DMF or DCM, affords a compound of formula I-5.

This invention also relates to a process for the preparation of a compound of formula (I) comprising at least one of the following steps:
(a) Substitution of a compound of formula (V),

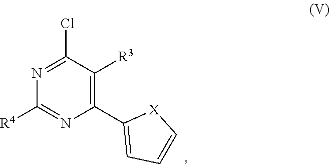

with a compound of formula (III),

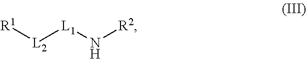

in the presence of a base;
(b) Coupling of a compound of formula (IV),

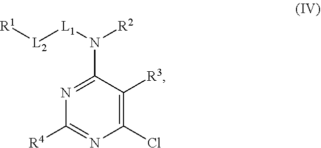

with a compound of formula (II),

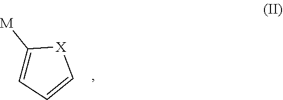

in the presence of Pd catalyst;
(c) Substitution of a compound of formula (XV).

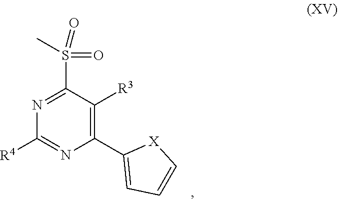

with a compound of formula (III), in the presence of a base;
(d) Substitution of a compound of formula (XII),

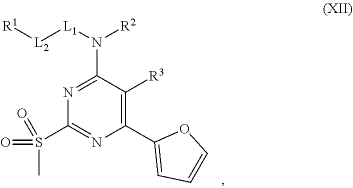

with a compound of formula (XII-1), R⁴H (XII-1);
(e) Hydrolysis of a compound of formula (I-3),

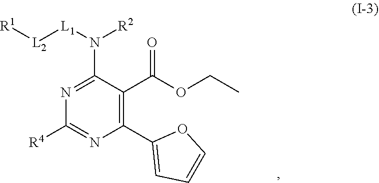

in the presence of a base;
(f) Coupling of a compound of formula (I-4),

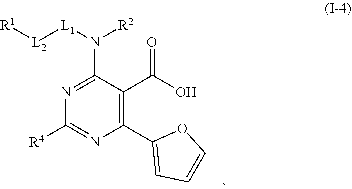

with a compound of formula (XVI), R⁵H (XVI), in the presence of a coupling reagent and a base;
wherein R⁵ is C₁₋₆ alkoxy, 4-6 membered heterocyclyl or C₁₋₆ alkoxycarbonyl(4-6 membered heterocyclyl); M is B(OH)₂ or SnBu₃.
The base in step (a) can be, for example, triethylamine, DIPEA or K₂CO₃.
The base in step (c) can be, for example, K₂CO₃, DIPEA or triethyl amine.
The base in step (e) can be, for example, KOH, LiOH or NaOH.
The coupling reagent in step (f) can be, for example, EDCI, HATU or T₃P.
The base in step (f) can be, for example, DMAP, TEA or DIPEA.

A compound of formula (I) when manufactured according to the above process is also an object of the invention.

### Pharmaceutical Compositions and Administration

The invention also relates to a compound of formula (I) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit cccDNA in HBV patients, consequently lead to the reduction of HBsAg and HBeAg (HBV e antigen) in serum. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of Formula (I), or pharmaceutically acceptable salt thereof.

In a further embodiment includes a pharmaceutical composition comprising a compound of formula (I), or pharmaceutically acceptable salt thereof, together with a pharmaceutically acceptable carrier or excipient.

Another embodiment includes a pharmaceutical composition comprising a compound of formula (I), or pharmaceutically acceptable salt thereof, for use in the treatment of HBV infection.

### Indications and Methods of Treatment

The compounds of the invention can inhibit cccDNA and have anti-HBV activity. Accordingly, the compounds of the invention are useful for the treatment or prophylaxis of HBV infection.

The invention relates to the use of a compound of formula (I) for the inhibition of cccDNA.

The invention also relates to the use of a compound of formula (I) for the inhibition of HBeAg.

The invention further relates to the use of a compound of formula (I) for the inhibition of HBsAg.

The invention relates to the use of a compound of formula (I) for the inhibition of HBV DNA.

The invention relates to the use of a compound of formula (I) for use in the treatment or prophylaxis of HBV infection.

The use of a compound of formula (I) for the preparation of medicaments useful in the treatment or prophylaxis diseases that are related to HBV infection is an object of the invention.

The invention relates in particular to the use of a compound of formula (I) for the preparation of a medicament for the treatment or prophylaxis of HBV infection.

Another embodiment includes a method for the treatment or prophylaxis of HBV infection, which method comprises administering an effective amount of a compound of Formula (I), or a pharmaceutically acceptable salts thereof.

### EXAMPLES

The invention will be more fully understood by reference to the following examples. They should not, however, be construed as limiting the scope of the invention.

Abbreviations used herein are as follows:
CDCl₃: deuterated chloroform
CD₃OD: deuterated methanol
DIPEA: N,N-diisopropylethylamine
DMF: dimethylformamide
DMSO-d₆: deuterated dimethylsulfoxide
EtOAc: ethyl acetate
EDCI: 1-(3-Dimethylaminopropyl)-3-ethylcarbodiimide Hydrochloride
HATU O-(7-aza-1H-benzotriazole-1-yl)-N,N,N′,N^{′}-tetramethyluronium hexafluorophosphate
HPLC: high performance liquid chromatography
Hr (hrs): hour (hours)
IC₅₀: the half maximal inhibitory concentration
LC/MS: Liquid chromatography/mass spectrometry
MeOH: methanol
M: molarity
m-CPBA: 3-chloroperoxybenzoic acid
MHz: megahertz
min: minute
mL: milliliter
mmol: millimole
MS (ESI⁺): mass spectroscopy (electron spray ionization)
NMP: N-methyl pyrrolidone
NMR: nuclear magnetic resonance
obsd. Observed
PPh₃: triphenylphosphine
rt: room temperature
SFC: supercritical fluid chromatography
TEA: triethylamine
TFA: trifluoroacetic acid
THF: tetrahydrofuran
TLC: thin layer chromatography
TMSI: iodotrimethylsilane
T₃P: 2,4,6-tripropyl-1,3,5,2,4,6-trioxatriphosphinane 2,4,6-trioxide
δ: chemical shift

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

LC/MS spectra were obtained using an Acquity Ultra Performance LC—3100 Mass Detector or Acquity Ultra Performance LC-SQ Detector. Standard LC/MS conditions were as follows (running time 3 minutes):

Acidic condition: A: 0.1% formic acid in H₂O; B: 0.1% formic acid in acetonitrile;

Basic condition: A: 0.05% NH₃.H₂O in H₂O; B: acetonitrile;

Neutral condition: A: H₂O; B: acetonitrile.

Mass spectra (MS): generally only ions which indicate the parent mass are reported, and unless otherwise stated the mass ion quoted is the positive mass ion (M+H)⁺.

The microwave assisted reactions were carried out in a Biotage Initiator Sixty or CEM Discover.

NMR Spectra were obtained using Bruker Avance 400 MHz.

All reactions involving air-sensitive reagents were performed under an argon atmosphere. Reagents were used as received from commercial suppliers without further purification unless otherwise noted.

### PREPARATIVE EXAMPLES

The invention will be more fully understood by reference to the following examples. They should not, however, be construed as limiting the scope of the invention.

### Intermediate Int-1

### 2-amino-4-chloro-6-(2-furyl)pyrimidine-5-carbonitrile

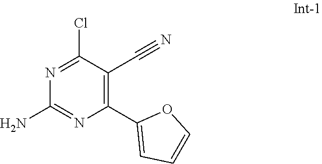 [SMILES: N#Cc1c(Cl)nc(N)nc1-c1ccco1]

The title compound was-prepared according to the following scheme

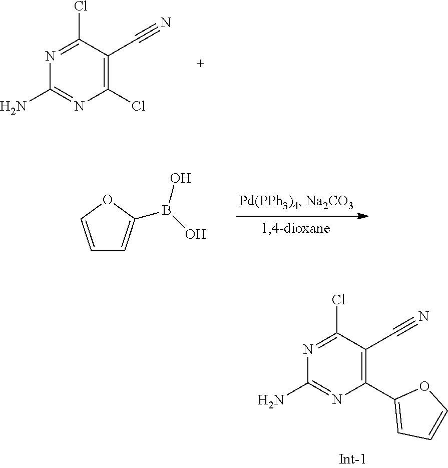 [SMILES: N#Cc1c(Cl)nc(N)nc1Cl ; OB(O)c1ccco1 ; N#Cc1c(Cl)nc(N)nc1-c1ccco1]

### Preparation of 2-amino-4-chloro-6-(2-furyl)pyrimidine-5-carbonitrile

To a mixture of 2-amino-4,6-dichloropyrimidine-5-carbonitrile (500 mg, 2.65 mmol) and furan-2-yl boronic acid (311 mg, 2.78 mmol) in dioxane (5 mL) was added Pd(Ph₃P)₄ (917 mg) and Na₂CO₃ (2.8 g, 2.65 mmol). After being stirred at 110° C. for 4 hrs, the reaction mixture was poured into 15 mL H₂O and extracted with DCM (3×15 mL). The organic layers were combined and dried over MgSO₄ and concentrated in vacuo. The crude material was purified by flash chromatography (silica gel, 40 g, 0% to 5% MeOH in DCM) to afford Int-1 (430 mg, 73.7%) as a yellow solid. MS obsd. (ESI⁺) [(M+H)⁺]: 221.

### Intermediate Int-2

### 4-chloro-6-(2-furyl)pyrimidine-5-carbonitrile

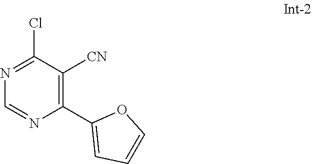 [SMILES: N#Cc1c(Cl)ncnc1-c1ccco1]

The title compound was prepared according to the following scheme:

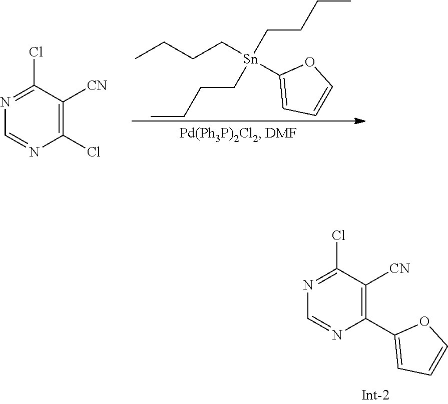 [SMILES: N#Cc1c(Cl)ncnc1Cl ; CCC[CH2][Sn]([CH2]CCC)([CH2]CCC)[c]1ccco1 ; N#Cc1c(Cl)ncnc1-c1ccco1]

### Preparation of 4-chloro-6-(2-furyl)pyrimidine-5-carbonitrile (Int-2)

To a mixture of 4,6-dichloropyrimidine-5-carbonitrile (5.85 g, 33.6 mmol) in DMF (25 mL) was added bis(triphenylphosphine)palladium(II) chloride (0.35 g, 0.5 mmol) and tributyl(2-furyl)stannane (5.29 mL, 16.8 mmol) under N₂. The mixture was stirred at rt for 16 hrs. After completion, the mixture was pour into saturated KF (50 mL) and EtOAc (50 mL) and filtered. The filtrate was concentrated and purified by flash column (eluting with EtOAc/PE=10%) to afford Int-2 (1.8 g, 26%). MS obsd. (ESI⁺) [(M+H)⁺]: 206.0.

### Intermediate Int-3

### 4-chloro-6-(2-furyl)-N,N-dimethyl-pyrimidin-2-amine

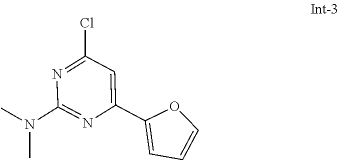

The title compound was prepared in analogy to the procedure described for the preparation of Int-1, by using 4,6-dichloro-N,N-dimethyl-pyrimidin-2-amine instead of 2-amino-4,6-dichloropyrimidine-5-carbonitrile. MS obsd. (ESI⁺) [(M+H)⁺]: 224.

### Intermediate Int-4

### 4-chloro-6-(2-furyl)-2-methoxy-pyrimidine

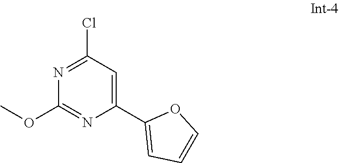

The title compound was prepared in analogy to the procedure described for the preparation of Int-1, by using 4,6-dichloro-2-methoxy-pyrimidine instead of 2-amino-4,6-dichloropyrimidine-5-carbonitrile. MS obsd. (ESI⁺) [(M+H)⁺]: 211.

### Intermediate Int-5

### 4-chloro-6-(2-furyl)-2-methyl-pyrimidine-5-carbonitrile

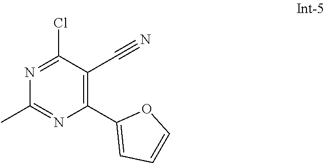

The title compound was prepared in analogy to the procedure described for the preparation of Int-1, by using 4,6-dichloro-2-methyl-pyrimidine instead of 2-amino-4,6-dichloropyrimidine-5-carbonitrile. MS obsd. (ESI⁺) [(M+H)⁺]: 220.

### Intermediate Int-6

### 4-chloro-6-(2-furyl)-5-methoxy-pyrimidin-2-amine

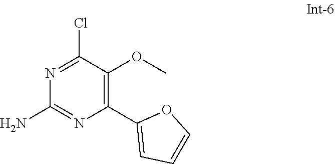

The title compound was prepared in analogy to the procedure described for the preparation of Int-1, by using 4,6-dichloro-5-methoxy-pyrimidin-2-amine instead of 2-amino-4,6-dichloropyrimidine-5-carbonitrile. MS obsd. (ESI⁺) [(M+H)⁺]: 226.

### Intermediate Int-7

### 4-chloro-6-(2-furyl)-2-methylsulfanyl-pyrimidine-5-carbonitrile

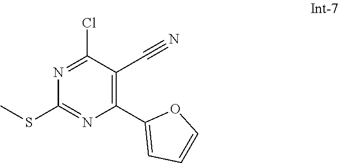 [SMILES: CSc1nc(Cl)c(C#N)c(-c2ccco2)n1]

The title compound was prepared in analogy to the procedure described for the preparation of Int-1, by using 4,6-dichloro-2-methylsulfanyl-pyrimidine-5-carbonitrile instead of 2-amino-4,6-dichloropyrimidine-5-carbonitrile. MS obsd. (ESI⁺) [(M+H)⁺]: 251.

### Intermediate Int-8

### Ethyl 4-chloro-6-(2-furyl)-2-methylsulfanyl-pyrimidine-5-carboxylate

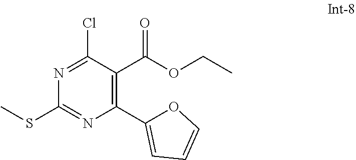 [SMILES: CCOC(=O)c1c(Cl)nc(SC)nc1-c1ccco1]

The title compound was prepared in analogy to the procedure described for the preparation of Int-1, by using ethyl 4,6-dichloro-2-methylsulfanyl-pyrimidine-5-carboxylate instead of 2-amino-4,6-dichloropyrimidine-5-carbonitrile. MS obsd. (ESI⁺) [(M+H)⁺]: 299.

### Intermediate Int-9

### 2-amino-4-(2-furyl)-6-methylsulfonyl-pyrimidine-5-carbonitrile

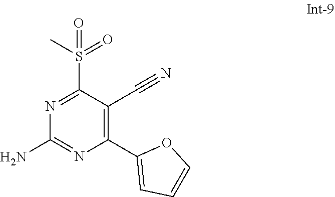 [SMILES: CS(=O)(=O)c1nc(N)nc(-c2ccco2)c1C#N]

The title compound was prepared according to the following scheme:

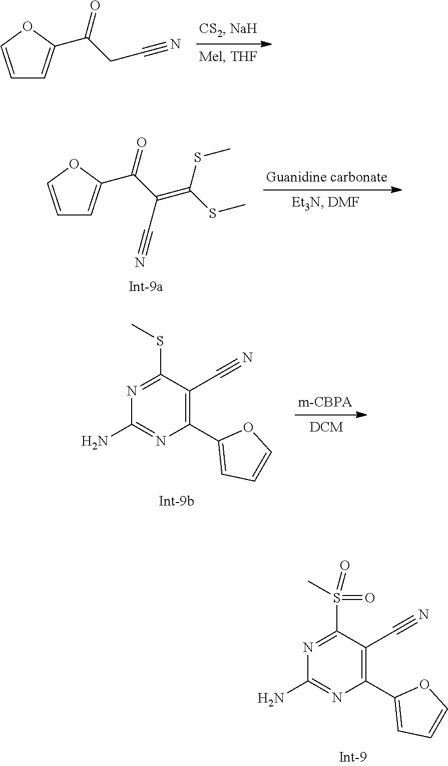 [SMILES: N#CCC(=O)c1ccco1 ; CSC(SC)=C(C#N)C(=O)c1ccco1 ; CSc1nc(N)nc(-c2ccco2)c1C#N ; CS(=O)(=O)c1nc(N)nc(-c2ccco2)c1C#N]

### Step 1: Preparation of 2-(furan-2-carbonyl)-3,3-bis(methylsulfanyl)prop-2-ene nitrile

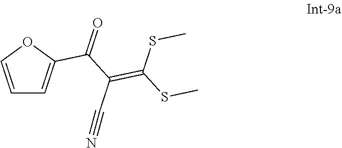

To a stirred solution of NaH (2.37 g, 59.2 mmol) in THF (200 ml) at 0° C. was added 3-(furan-2-yl)-3-oxopropanenitrile (4 g, 29.6 mmol) dropwise, the suspension was stirred for 20 min, and carbon disulfide (2.48 g, 2.02 ml, 32.6 mmol) was added dropwise. The reaction mixture was allowed to stir for 30 min, and Mel (9.24 g, 4.07 ml, 65.1 mmol) was added dropwise. The mixture was allowed to rt overnight. The reaction mixture was poured into ice water and was extracted with EtOAc, dried over anhydrous sodium sulfate, and the solvent was distilled under reduced pressure and the dithioacetal body was obtained. Recrystallization from n-hexane and EtOAc afforded Int-8a (5.7 g, 80.5%) as a light brown solid. MS obsd. (ESI⁺) [(M+H)⁺]: 240.

### Step 2: Preparation of 2-amino-4-(2-furyl)-6-methylsulfanyl-pyrimidine-5-carbonitrile

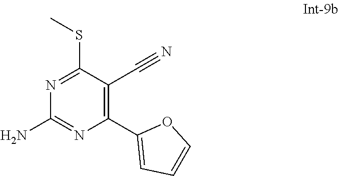 [SMILES: CSc1nc(N)nc(-c2ccco2)c1C#N]

To a solution of 2-(furan-2-carbonyl)-3,3-bis(methylthio)acrylonitrile (Int-9a, 2 g, 8.36 mmol) and guanidine carbonate (1.81 g, 10 mmol) in DMF (20 mL) was added Et₃N (2.11 g, 2.91 mL, 20.9 mmol). After being heated at reflux for 2 hrs, the reaction mixture was then cooled to rt. After addition of 30 mL water, a precipitate appeared which was collected by filtration and washed with water to afford Int-8b (1.5 g, 77.3%) as a yellow solid. MS obsd. (ESI⁺) [(M+H)⁺]: 233.

### Step 3: Preparation of 2-amino-4-(2-furyl)-6-methylsulfonyl-pyrimidine-5-carbonitrile

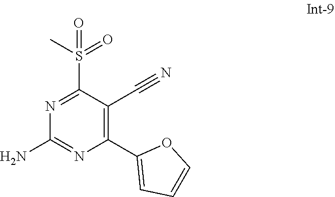 [SMILES: CS(=O)(=O)c1nc(N)nc(-c2ccco2)c1C#N]

To a solution of 2-amino-4-(furan-2-yl)-6-(methylthio)pyrimidine-5-carbonitrile (Int-9b, 1.5 g, 6.46 mmol) in CH₂Cl₂ (50 ml) was added m-CPBA (3.48 g, 16.1 mmol). After being stirred at 25° C. for 1 hr, the volume of reaction mixture was reduced in vacuo. The crude material was purified by flash chromatography (silica gel, 40 g, 0% to 40% EtOAc in hexanes) to afford Int-9 (1.5 g, 87.9%) as a yellow solid. MS obsd. (ESI⁺) [(M+H)⁺]: 265.

### Intermediate Int-10

### Ethyl 2-amino-4-(2-furyl)-6-methylsulfonyl-pyrimidine-5-carboxylate

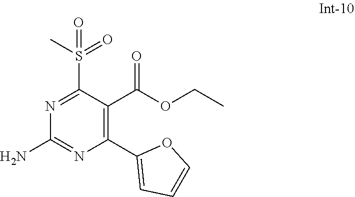 [SMILES: CCOC(=O)c1c(-c2ccco2)nc(N)nc1S(C)(=O)=O]

The title compound was prepared according to the following scheme:

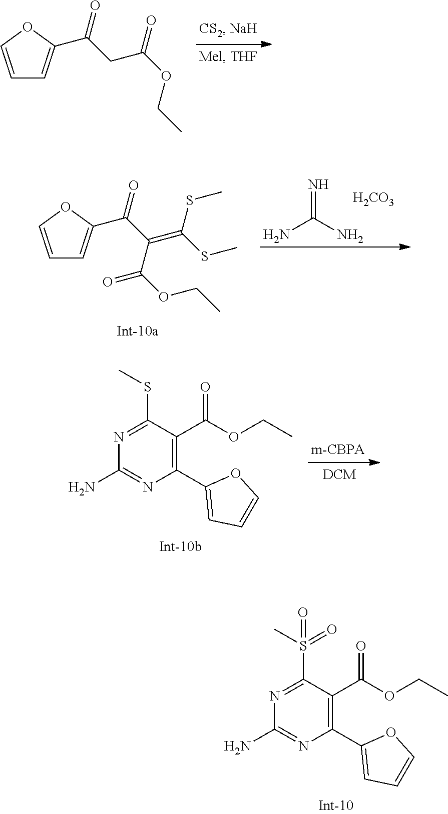

### Step 1: Preparation of ethyl 2-(furan-2-carbonyl)-3,3-bis(methylsulfanyl)prop-2-enoate

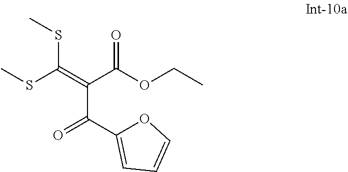 [SMILES: CCOC(=O)C(C(=O)c1ccco1)=C(SC)SC]

The title compound was prepared in analogy to the procedure described for the preparation of Int-9a, by using ethyl 3-(2-furyl)-3-oxo-propanoate instead of 23-(furan-2-yl)-3-oxopropanenitrile. The product was purified by preparative HPLC to afford Int-10a (1.33 g, 84.86%) as light yellow oil. MS obsd. (ESI⁺)[(M+H)⁺]: 309.0.

### Step 2: Preparation of ethyl 2-amino-4-(2-furyl)-6-methylsulfanyl-pyrimidine-5-carboxylate

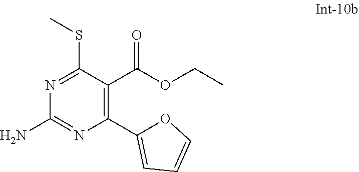 [SMILES: CCOC(=O)c1c(SC)nc(N)nc1-c1ccco1]

The title compound was prepared in analogy to the procedure described for the preparation of Int-9b, by using ethyl 2-(furan-2-carbonyl)-3,3-bis(methylsulfanyl)prop-2-enoate (Int-10a) instead of 2-(furan-2-carbonyl)-3,3-bis(methylthio)acrylonitrile (Int-9a). The product was purified by preparative HPLC to afford Int-10b (1.5 g, 5.37 mmol, 76.9%) as a white solid. MS obsd. (ESI⁺)[(M+H)⁺]: 280.1.

### Step 3: Preparation of ethyl 2-amino-4-(2-furyl)-6-methylsulfonyl-pyrimidine-5-carboxylate

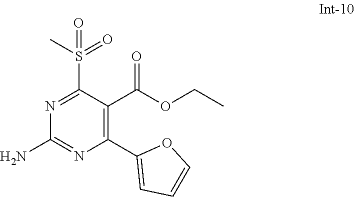 [SMILES: CCOC(=O)c1c(-c2ccco2)nc(N)nc1S(C)(=O)=O]

The title compound was prepared in analogy to the procedure described for the preparation of Int-9, by using ethyl 2-amino-4-(2-furyl)-6-methylsulfanyl-pyrimidine-5-carboxylate (Int-10b) instead of 2-amino-4-(furan-2-yl)-6-(methylthio)pyrimidine-5-carbonitrile (Int-9b). The product was purified by preparative HPLC to afford Int-10 (1.0 g, 44.9%) as a white solid. MS obsd. (ESI⁺)[(M+H)⁺]: 312.0. ¹H NMR (400 MHz, CDCl₃) δ ppm: 7.60 (s, 1H), 7.29 (d, J=3.5 Hz, 1H), 6.58 (dd, J=3.5, 1.6 Hz, 1H), 5.63 (s, 2H), 4.45 (q, J=7.2 Hz, 2H), 3.23 (s, 3H), 1.39 (t, J=7.2 Hz, 3H).

### Intermediate-11

### Ethyl 4-chloro-6-(2-furyl)-2-methyl-pyrimidine-5-carboxylate

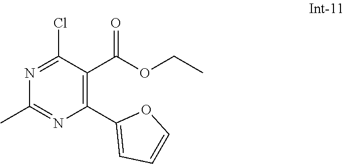 [SMILES: CCOC(=O)c1c(Cl)nc(C)nc1-c1ccco1]

The title compound was prepared according to the following scheme:

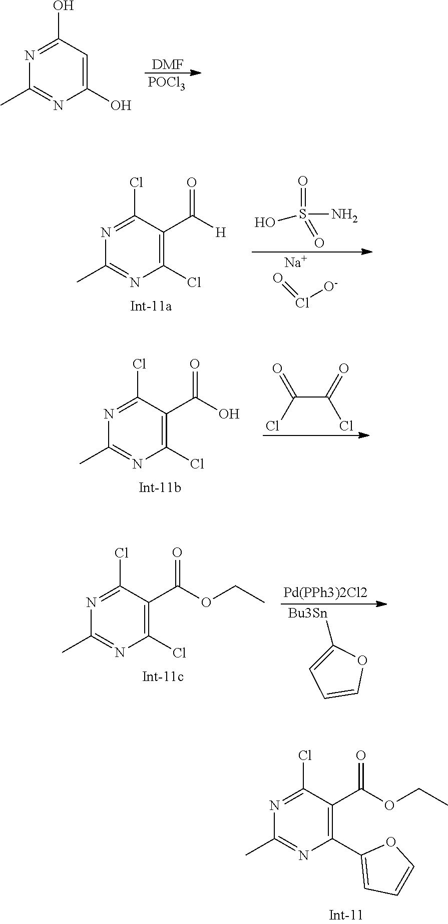

### Step 1: Preparation of 4,6-dichloro-2-methyl-pyrimidine-5-carbaldehyde

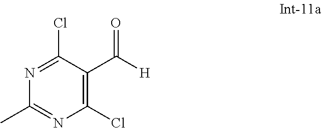

To a solution of phosphorus oxychloride (31.69 mL, 340 mmol) at 0° C. was added dimethylformamide (3.1 mL, 40 mmol) dropwise. After addition, the mixture was stirred at 25° C. for 1 hr, 4,6-dihydroxy-2-methylpyrimidine (5.04 g, 40 mmol) was added in small portions. The resulting yellow suspension was stirred at 120° C. for 16 hrs. After cooling to rt, the mixture was poured into ice (200 g) and then diluted with diethyl ether/EtOAc=4/1 (150 mL). The insoluble material was filtered off. The filtrate was extracted with diethyl ether/EtOAc=4/1 (100 mL×3). The combined organic layers were washed with water (100 mL), brine (100 mL), dried over anhydrous Na₂SO₄, filtered and concentrated in vacuo. The residue was purified by flash silica gel column chromatography (eluting with EtOAc/PE=5%) to afford Int-11a (2.1 g, 10.99 mmol, 25.94%) as a yellow solid. MS obsd. (ESI⁺)[(M+H)⁺]: 191.1.

### Step 2: Preparation of 4,6-dichloro-2-methyl-pyrimidine-5-carboxylic Acid

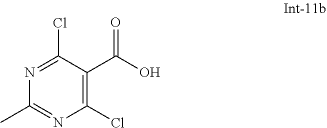

To a solution of 4,6-dichloro-2-methyl-pyrimidine-5-carbaldehyde (0.96 g, 4.72 mmol), sulfamic acid (0.58 mL, 5.66 mmol) in water (5 mL) and tert-butanol (20 mL) in a water bath was added a solution of sodium chlorite (7.92 mL, 5.66 mmol) in water (5 mL) dropwise (a 2-5° C. exotherm was observed during the addition). After being stirred at 25° C. for 3 hrs, the mixture was poured into water (100 mL) and extracted with EtOAc (50 mL×3). The combined organic layers were washed with brine (50 mL), dried over anhydrous Na₂SO₄, filtered and concentrated in vacuo to afford Int-11b (1 g, 88.26%) as a yellow solid.

### Step 3: Preparation of ethyl 4,6-dichloro-2-methyl-pyrimidine-5-carboxylate

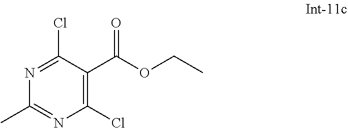

To a solution of 4,6-dichloro-2-methyl-pyrimidine-5-carboxylic acid (1.0 g, 4.16 mmol) in dry THE (10 mL) at 25° C. was added oxalyl chloride (0.84 mL, 6.25 mmol) dropwise. 3 drops of DMF was added successively. After being stirred at 25° C. for 2 hrs, the mixture was concentrated in vacuo to give a yellow oil. The residue was diluted with ethanol (10 mL), added with triethylamine (0.87 mL, 6.25 mmol) dropwise. The resulting brown mixture was stirred at 25° C. for 16 hrs. Then mixture was concentrated in vacuo and purified by silica gel chromatography (EtOAc/PE=5/95) to afford Int-11c (680 mg) as a white solid. MS obsd. (ESI⁺)[(M+H)⁺]: 235.1.

### Step 4: Preparation of ethyl 4-chloro-6-(2-furyl)-2-methyl-pyrimidine-5-carboxylate

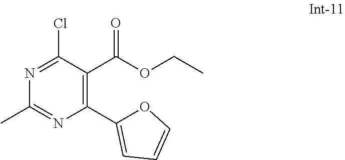 [SMILES: CCOC(=O)c1c(Cl)nc(C)nc1-c1ccco1]

The title compound was prepared in analogy to the procedure described for the preparation of Int-2a, by using ethyl 4,6-dichloro-2-methyl-pyrimidine-5-carboxylate (Int-11c) instead of 4,6-dichloropyrimidine-5-carbonitrile. The product was purified by preparative HPLC to afford Int-11 (60 mg, 17.65%) as a yellow solid. MS (ESI⁺) [(M+H)⁺]: 267.0.

### Intermediate Int-12

### Ethyl 4-chloro-6-(2-furyl)-2-isopropyl-pyrimidine-5-carboxylate

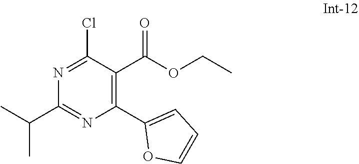 [SMILES: CCOC(=O)c1c(Cl)nc(C(C)C)nc1-c1ccco1]

The title compound was prepared according to the following scheme:

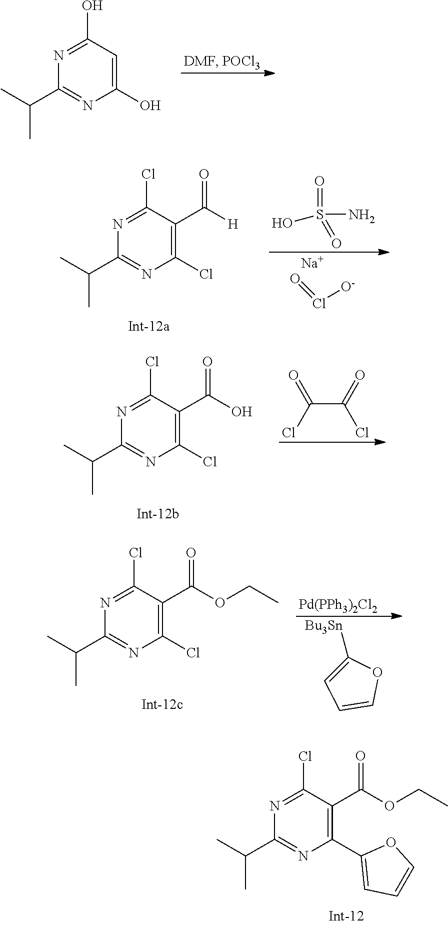

### Step 1: Preparation of 4,6-dichloro-2-isopropyl-pyrimidine-5-carbaldehyde

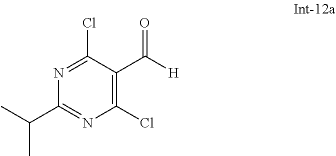

The title compound was prepared in analogy to the procedure described for the preparation of Int-11a, by using 2-isopropylpyrimidine-4,6-diol instead of 4,6-dihydroxy-2-methylpyrimidine. The product was purified by preparative HPLC to afford Int-12a (2.11 g, 48.21%) as a white solid. MS (ESI⁺) [(M+H)⁺]: 219.1.

### Step 2: Preparation of 4,6-dichloro-2-isopropyl-pyrimidine-5-carboxylic Acid

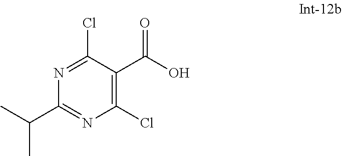

The title compound was prepared in analogy to the procedure described for the preparation of Int-11b, by using 4,6-dichloro-2-isopropyl-pyrimidine-5-carbaldehyde (Int-12a) instead of 4,6-dichloro-2-methyl-pyrimidine-5-carbaldehyde (Int-11a). The product was purified by preparative HPLC to afford Int-12b (1.3 g) as a yellow solid. MS (ESI⁺) [(M+H)⁺]: 235.0.

### Step 3: Preparation of ethyl 4,6-dichloro-2-isopropyl-pyrimidine-5-carboxylate

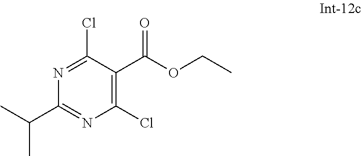 [SMILES: CCOC(=O)c1c(Cl)nc(C(C)C)nc1Cl]

The title compound was prepared in analogy to the procedure described for the preparation of Int-11c, by using 4,6-dichloro-2-isopropyl-pyrimidine-5-carboxylic acid (Int-12b) instead of 4,6-dichloro-2-methyl-pyrimidine-5-carboxylic acid (Int-11b). Ethyl 4,6-dichloro-2-isopropyl-pyrimidine-5-carboxylate (Int-12c, 590 mg) was obtained as a colorless oil. MS obsd. (ESI⁺)[(M+H)⁺]: 263.1 Step 4: Preparation of ethyl 4-chloro-6-(2-furyl)-2-isopropyl-pyrimidine-5-carboxylate

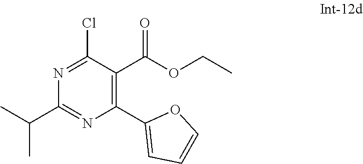 [SMILES: CCOC(=O)c1c(Cl)nc(C(C)C)nc1-c1ccco1]

The title compound was prepared in analogy to the procedure described for the preparation of Int-11d, by using ethyl 4,6-dichloro-2-isopropyl-pyrimidine-5-carboxylate (Int-12c) instead of ethyl 4,6-dichloro-2-methyl-pyrimidine-5-carboxylate (Int-11c). Ethyl 4-chloro-6-(2-furyl)-2-isopropyl-pyrimidine-5-carboxylate (Int-12d, 600 mg) was obtained as a colorless oil. MS obsd. (ESI⁺)[(M+H)⁺]: 295.1.

### Example 1

### 2-amino-4-(2-furyl)-6-(1-phenylethylamino)pyrimidine-5-carbonitrile

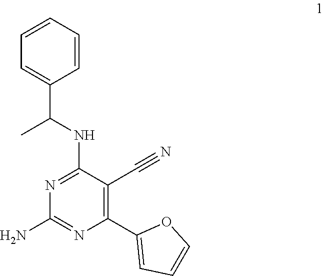 [SMILES: CC(Nc1nc(N)nc(-c2ccco2)c1C#N)c1ccccc1]

To a 10 mL microwave vial was added 2-amino-4-chloro-6-(2-furyl)pyrimidine-5-carbonitrile (Int-1, 6 mg), 1-phenylethanamine (6.59 mg) and K₂CO₃ (7.52 mg) in DMF (1 mL). The vial was capped and heated in the microwave at 150° C. for 30 min. After filtration, the crude filtrate was purified by preparative HPLC to afford Example 1 (4.7 mg, 56%) as a white solid. MS obsd. (ESI⁺) [(M+H)⁺]: 306. ¹H NMR (400 MHz, DMSO-d₆) δ ppm: 7.95 (dd, J=1.7, 0.7 Hz, 1H), 7.44 (d, J=7.9 Hz, 3H), 7.30-7.35 (m, 2H), 7.28 (dd, J=3.5, 0.6 Hz, 1H), 7.20-7.25 (m, 1H), 7.14 (br s, 1H), 7.02 (br s, 1H), 6.71 (dd, J=3.5, 1.8 Hz, 1H), 5.45 (quin, J=7.4 Hz, 1H), 1.53 (d, J=7.1 Hz, 3H).

### Example 2

### 2-amino-4-[(3-bromo-4-isopropoxy-phenyl)methylamino]-6-(2-furyl)pyrimidine-5-carbonitrile

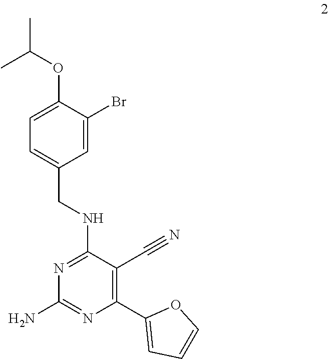 [SMILES: CC(C)Oc1ccc(CNc2nc(N)nc(-c3ccco3)c2C#N)cc1Br]

The title compound was prepared in analogy to the procedure described for the preparation of Example 1, by using (3-bromo-4-isopropoxyphenyl)methanamine hydrochloride instead of 1-phenylethanamine. The product was purified by preparative HPLC to afford Example 2 (6.7 mg, 34%) as a white solid. MS obsd. (ESI⁺) [(M+H)⁺]: 428. ¹H NMR (400 MHz, DMSO-d₆) δ ppm: 7.97 (d, J=1.0 Hz, 1H), 7.90 (br s, 1H), 7.56 (d, J=2.1 Hz, 1H), 7.25-7.36 (m, 2H), 7.20 (br d, J=7.8 Hz, 2H), 7.07 (d, J=8.7 Hz, 1H), 6.73 (dd, J=3.5, 1.8 Hz, 1H), 4.61 (dt, J=12.1, 6.0 Hz, 1H), 4.50 (d, J=6.1 Hz, 2H), 1.28 (s, 3H), 1.26 (s, 3H).

### Example 3

### 2-amino-4-(2-furyl)-6-[(2,2,6,6-tetramethyltetrahydropyran-4-yl)methylamino]pyrimidine-5-carbonitrile

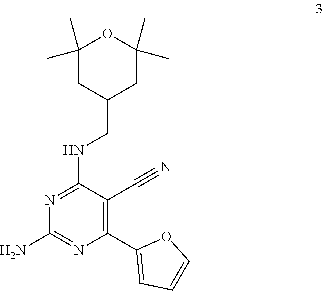 [SMILES: CC1(C)CC(CNc2nc(N)nc(-c3ccco3)c2C#N)CC(C)(C)O1]

The title compound was prepared in analogy to the procedure described for the preparation of Example 1, by using (2,2,6,6-tetramethyltetrahydro-2H-pyran-4-yl)methanamine hydrochloride instead of 1-phenylethanamine. The product was purified by preparative HPLC to afford Example 3 (5.8 mg, 24%) as a white solid. MS obsd. (ESI⁺) [(M+H)⁺]: 356. ¹H NMR (400 MHz, DMSO-d₆) δ ppm: 7.99 (d, J=1.0 Hz, 1H), 7.39 (br s, 1H), 7.33 (d, J=3.3 Hz, 1H), 7.20 (br d, J=13.4 Hz, 2H), 6.74 (dd, J=3.5, 1.7 Hz, 1H), 3.19-3.38 (m, 2H), 2.21-2.35 (m, 1H), 1.56 (dd, J=13.0, 2.9 Hz, 2H), 1.18 (s, 6H), 1.10 (s, 6H), 0.89 (t, J=12.7 Hz, 2H).

### Example 4

### 2-amino-4-[[3-(2-chlorophenyl)-2-fluoro-propyl]amino]-6-(2-furyl)pyrimidine-5-carbonitrile

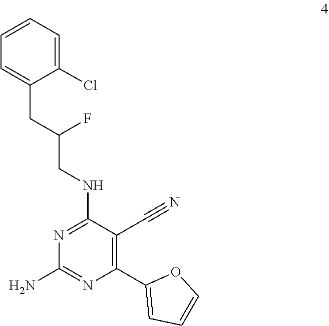 [SMILES: N#Cc1c(NCC(F)Cc2ccccc2Cl)nc(N)nc1-c1ccco1]

The title compound was prepared in analogy to the procedure described for the preparation of Example 1, by using 3-(2-chlorophenyl)-2-fluoropropan-1-amine hydrochloride instead of 1-phenylethanamine. The product was purified by preparative HPLC to afford Example 4 (2 mg, 8%) as an off-white solid. MS obsd. (ESI⁺) [(M+H)⁺]: 372. ¹H NMR (400 MHz, DMSO-d₆) δ ppm: 7.96 (d, J=1.0 Hz, 1H), 7.40-7.54 (m, 4H), 7.25-7.35 (m, 4H), 6.73 (dd, J=3.5, 1.8 Hz, 1H), 4.89-5.13 (m, 1H), 3.53-3.83 (m, 2H), 3.48-3.66 (m, 2H), 2.94-3.23 (m, 2H).

### Example 5

### 2-amino-4-(2-furyl)-6-[1-[3-(trifluoromethyl)phenyl]ethylamino]pyrimidine-5-carbonitrile

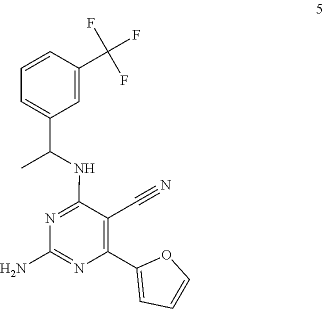 [SMILES: CC(Nc1nc(N)nc(-c2ccco2)c1C#N)c1cccc(C(F)(F)F)c1]

The title compound was prepared in analogy to the procedure described for the preparation of Example 1, by using 3-(2-chlorophenyl)-2-fluoropropan-1-amine hydrochloride instead of 1-phenylethanamine. The product was purified by preparative HPLC to afford Example 5 (5.3 mg, 17%) as an off-white solid. MS obsd. (ESI⁺) [(M+H)⁺]: 374. ¹H NMR (400 MHz, DMSO-d₆) S ppm: 7.95 (dd, J=1.6, 0.7 Hz, 1H), 7.84 (s, 1H), 7.76 (d, J=7.3 Hz, 1H), 7.69 (d, J=8.1 Hz, 1H), 7.54-7.61 (m, 2H), 7.28 (dd, J=3.5, 0.7 Hz, 1H), 7.19 (br s, 1H), 7.04 (br s, 1H), 6.71 (dd, J=3.5, 1.7 Hz, 1H), 5.49 (quin, J=7.4 Hz, 1H), 1.56 (d, J=7.1 Hz, 3H).

### Example 6

### 6-(2-furyl)-N4-[(3-methoxyphenyl)methyl]-N4-methyl-pyrimidine-2,4-diamine

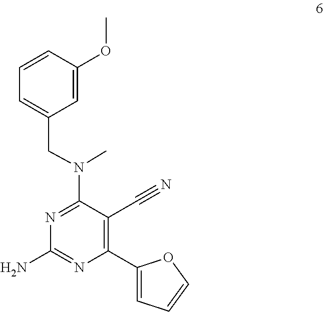 [SMILES: COc1cccc(CN(C)c2nc(N)nc(-c3ccco3)c2C#N)c1]

The title compound was prepared in analogy to the procedure described for the preparation of Example 1, by using 1-(3-methoxyphenyl)-N-methylmethanamine instead of 1-phenylethanamine. The product was purified by preparative HPLC to afford Example 6 (4 mg) as an off-white solid. MS obsd. (ESI⁺) [(M+H)⁺]: 336. ¹H NMR (400 MHz, METHANOL-d₄) δ=7.96-7.76 (m, 1H), 7.69-7.56 (m, 1H), 7.39-7.16 (m, 1H), 6.99-6.80 (m, 3H), 6.80-6.61 (m, 1H), 5.05-4.98 (m, 2H), 4.89 (s, 3H), 3.80 (s, 3H).

### Example 7

### 2-amino-4-(2-furyl)-6-[[1-(2-methylsulfonylphenyl)cyclopropyl]amino]pyrimidine-5-carbonitrile

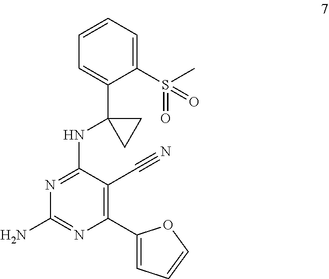 [SMILES: CS(=O)(=O)c1ccccc1C1(Nc2nc(N)nc(-c3ccco3)c2C#N)CC1]

The title compound was prepared in analogy to the procedure described for the preparation of Example 1, by using 2-amino-4-(2-furyl)-6-methylsulfonyl-pyrimidine-5-carbonitrile (Int-9) and 1-(2-(methylsulfonyl)phenyl)cyclopropan-1-amine hydrochloride instead of 2-amino-4-chloro-6-(furan-2-yl)pyrimidine-5-carbonitrile (Int-1) and 1-phenylethanamine. The product was purified by preparative HPLC to afford Example 7 (10.8 mg, 23.6%) as an off-white powder. MS obsd. (ESI⁺) [(M+H)⁺]: 396. ¹H NMR (400 MHz, DMSO-d₆) δ ppm: 8.33-8.17 (m, 1H), 8.00-7.88 (m, 2H), 7.71-7.59 (m, 1H), 7.59-7.48 (m, 1H), 7.38-7.17 (m, 3H), 7.17-7.09 (m, 1H), 6.73-6.62 (m, 1H), 3.35 (s, 3H), 1.56-1.15 (m, 4H).

### Example 8

### 2-amino-4-[(3,3-difluorocyclopentyl)methylamino]-6-(2-furyl)pyrimidine-5-carbonitrile

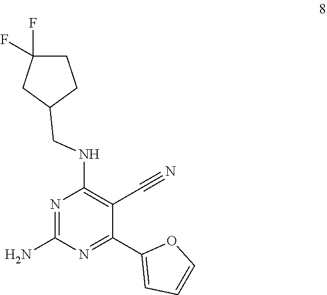 [SMILES: N#Cc1c(NCC2CCC(F)(F)C2)nc(N)nc1-c1ccco1]

The title compound was prepared in analogy to the procedure described for the preparation of Example 1), by using (3,3-difluorocyclopentyl)methanamine instead of 1-phenylethanamine. The product was purified by preparative HPLC to afford Example 8 (36.6 mg, 25.2%) as a white solid. MS obsd. (ESI⁺) [(M+H)⁺]: 320.1. ¹H NMR (400 MHz, CD₃OD) δ ppm: 7.76 (d, J=1.1 Hz, 1H), 7.41 (d, J=3.5 Hz, 1H), 6.65 (dd, J=3.5, 1.7 Hz, 1H), 3.50 (d, J=7.1 Hz, 2H), 2.48-2.62 (m, 1H), 1.78-2.28 (m, 6H), 1.50-1.64 (m, 1H). ¹H NMR (376 MHz, DMSO-d₆) δ ppm: −88.90 (q).

### Example 9

### 2-amino-4-(cyclopentylamino)-6-(2-furyl)pyrimidine-5-carbonitrile

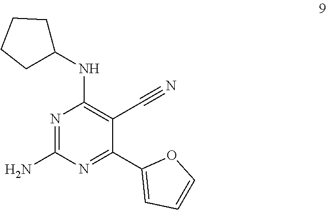 [SMILES: N#Cc1c(NC2CCCC2)nc(N)nc1-c1ccco1]

The title compound was prepared in analogy to the procedure described for the preparation of Example 1, by using cyclopentylamine instead of 1-phenylethanamine. The product was purified by preparative HPLC to afford Example 9 (26.8 mg, 43.91%) as a light yellow solid. MS obsd. (ESI⁺)[(M+H)⁺]: 270.1. ¹H NMR (400 MHz, CD₃OD) 5 ppm: 7.65 (d, J=1.0 Hz, 1H), 7.49 (d, J=3.5 Hz, 1H), 6.57 (dd, J=3.6, 1.7 Hz, 1H), 5.42 (d, J=6.6 Hz, 1H), 5.30 (s, 2H), 4.42 (dd, J=14.0, 7.0 Hz, 1H), 2.08 (dd, J=12.5, 5.5 Hz, 2H), 1.71-1.79 (m, 2H), 1.48 (m, 2H), 1.66 (m, 2H).

### Example 10

### 2-amino-4-(cyclohexylmethylamino)-6-(2-furyl)pyrimidine-5-carbonitrile

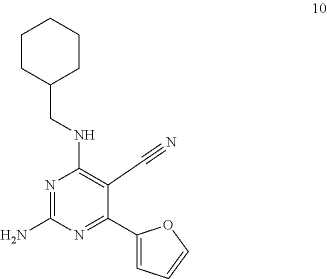 [SMILES: N#Cc1c(NCC2CCCCC2)nc(N)nc1-c1ccco1]

The title compound was prepared in analogy to the procedure described for the preparation of Example 1, by using cyclohexanemethylamine instead of 1-phenylethanamine. The product was purified by preparative HPLC to afford Example 10 (49.7 mg) as a white solid. MS obsd. (ESI⁺)[(M+H)⁺]: 298.1. ¹H NMR (400 MHz, DMSO-d₆) δ ppm: 7.94 (d, J=0.9 Hz, 1H), 7.26 (d, J=3.4 Hz, 1H), 7.16 (br, 2H) 7.20 (m, 1H), 6.70 (dd, J=3.5, 1.7 Hz, 1H), 3.25 (m, 2H), 1.66-1.69 (m, 6H), 1.17 (m, 3H), 0.89-0.91 (m, 2H).

### Example 11

### 4-(2-furyl)-2-methylsulfanyl-6-[[3-(trifluoromethyl)phenyl]methylamino]pyrimidine-5-carbonitrile

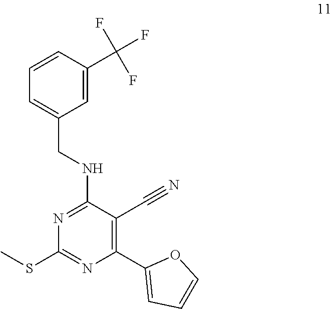 [SMILES: CSc1nc(NCc2cccc(C(F)(F)F)c2)c(C#N)c(-c2ccco2)n1]

The title compound was prepared in analogy to the procedure described for the preparation of Example 1, by using 4-chloro-6-(furan-2-yl)-2-(methylthio)pyrimidine-5-carbonitrile (Int-7) and (3-(trifluoromethyl)phenyl)methanamine instead of 2-amino-4-chloro-6-(2-furyl)pyrimidine-5-carbonitrile (Int-1) and 1-phenylethanamine. The product was purified by preparative HPLC to afford Example 11 (1 g) as an off-white powder. MS obsd. (ESI⁺) [(M+H)⁺]: 391. ¹H NMR (400 MHz, DMSO-d₆) δ ppm: 8.78-8.64 (m, 1H), 8.06 (s, 1H), 7.75 (s, 1H), 7.71-7.54 (m, 3H), 7.44 (d, J=3.4 Hz, 1H), 6.81-6.74 (m, 1H), 4.71 (d, J=5.9 Hz, 2H), 2.40 (s, 3H).

### Example 12

### 4-(2-furyl)-2-(2-methoxyethylamino)-6-[[3-(trifluoromethyl)phenyl]methylamino]pyrimidine-5-carbonitrile

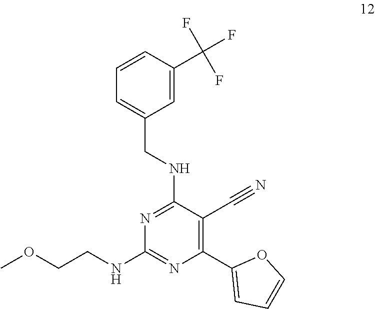 [SMILES: COCCNc1nc(NCc2cccc(C(F)(F)F)c2)c(C#N)c(-c2ccco2)n1]

The title compound was prepared according to the following scheme:

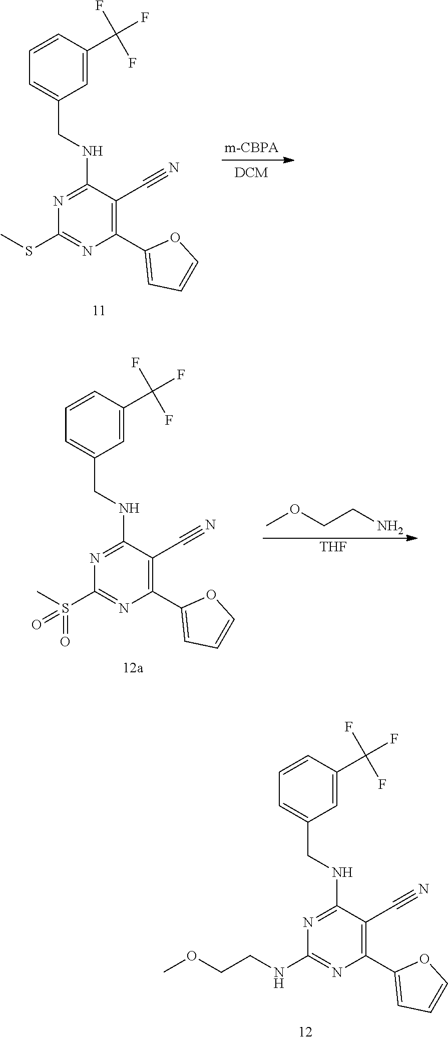

### Step 1: Preparation of 4-(2-furyl)-2-methylsulfonyl-6-[[3-(trifluoromethyl)phenyl]methylamino]pyrimidine-5-carbonitrile

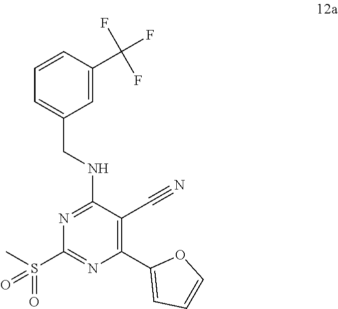 [SMILES: CS(=O)(=O)c1nc(NCc2cccc(C(F)(F)F)c2)c(C#N)c(-c2ccco2)n1]

To a solution of 4-(furan-2-yl)-2-(methylthio)-6-((3-(trifluoromethyl)benzyl)amino)pyrimidine-5-carbonitrile (Example 11, 140 mg) in CH₂Cl₂ (50 ml) was added 3-chlorobenzoperoxoic acid (221 mg). After being stirred at 25° C. for 1 hr, the volume of reaction mixture was reduced in vacuo. The crude material was purified by flash chromatography (silica gel, 40 g, 0% to 40% EtOAc in hexanes) to afford 4-(2-furyl)-2-methylsulfonyl-6-[[3-(trifluoromethyl)phenyl]methylamino]pyrimidine-5-carbonitrile (Compound 12a, 140 mg, 92.4%) as an off-white Solid. MS obsd. (ESI⁺) [(M+H)⁺]: 423.

### Step 2: Preparation of 4-(2-furyl)-2-(2-methoxyethylamino)-6-[[3-(trifluoromethyl)phenyl]methylamino]pyrimidine-5-carbonitrile

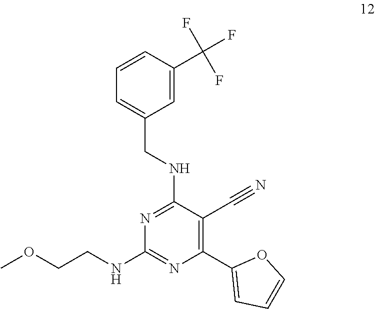 [SMILES: COCCNc1nc(NCc2cccc(C(F)(F)F)c2)c(C#N)c(-c2ccco2)n1]

A mixture of 4-(furan-2-yl)-2-(methylsulfonyl)-6-((3-(trifluoromethyl)benzyl)amino)pyrimidine-5-carbonitrile (Compound 12a, 50 mg) and 2-methoxyethan-1-amine (17.8 mg) in THF (1 ml) was stirred at rt for 2 hrs. The crude reaction mixture was concentrated in vacuo. The crude material was purified by preparative HPLC to afford Example 12 (35 mg) as a white powder. MS obsd. (ESI⁺) [(M+H)⁺]: 418. ¹H NMR (400 MHz, DMSO-d₆) δ ppm: 8.25 (br t, J=5.7 Hz, 1H), 7.97 (s, 1H), 7.76-7.69 (m, 2H), 7.65-7.54 (m, 3H), 7.36-7.28 (m, 1H), 6.73 (dd, J=1.7, 3.4 Hz, 1H), 4.65 (br d, J=5.9 Hz, 2H), 3.37-3.28 (m, 2H), 3.28-3.19 (m, 2H), 3.12 (s, 3H).

### Example 13

### 4-(2-furyl)-2-(2,2,2-trifluoroethylamino)-6-[[3-(trifluoromethyl)phenyl]methylamino]pyrimidine-5-carbonitrile

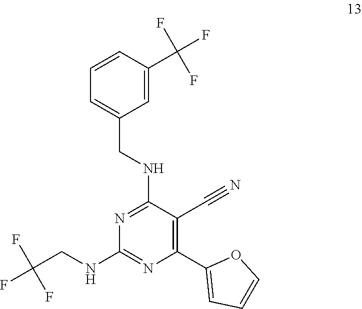 [SMILES: N#Cc1c(NCc2cccc(C(F)(F)F)c2)nc(NCC(F)(F)F)nc1-c1ccco1]

The title compound was prepared in analogy to the procedure described for the preparation of Example 12, by using 2,2,2-trifluoroethan-1-amine instead of 2-methoxyethan-1-amine. The product was purified by preparative HPLC to afford Example 13 (21 mg, 39.4%) as a light yellow powder. MS obsd. (ESI⁺) [(M+H)⁺]: 442. ¹H NMR (400 MHz, DMSO-d₆) δ ppm: 8.40-8.22 (m, 1H), 8.07-7.95 (m, 1H), 7.75-7.49 (m, 5H), 7.37-7.28 (m, 1H), 6.74 (dd, J=1.7, 3.4 Hz, 1H), 4.74-4.62 (m, 2H), 4.11-3.93 (m, 2H).

### Example 14

### 2-(ethylamino)-4-(2-furyl)-6-[[3-(trifluoromethyl)phenyl]methylamino]pyrimidine-5-carbonitrile

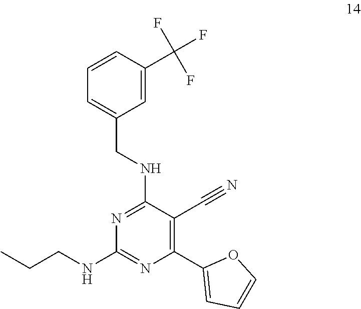 [SMILES: CCCNc1nc(NCc2cccc(C(F)(F)F)c2)c(C#N)c(-c2ccco2)n1]

The title compound was prepared in analogy to the procedure described for the preparation of Example 12, by using ethanamine instead of 2-methoxyethan-1-amine. The product was purified by preparative HPLC to afford Example 14 (16 mg, 34.2%) as a white powder. MS obsd. (ESI⁺) [(M+H)⁺]: 388. ¹H NMR (400 MHz, DMSO-d₆) δ ppm: 8.18 (s, 1H), 7.95 (s, 1H), 7.76-7.64 (m, 3H), 7.64-7.53 (m, 2H), 7.27 (d, J=3.4 Hz, 1H), 6.71 (dd, J=1.7, 3.2 Hz, 1H), 4.63 (br d, J=6.0 Hz, 2H), 3.28-3.15 (m, 2H), 0.94 (t, J=7.1 Hz, 3H).

### Example 15

### 4-(2-furyl)-2-(propylamino)-6-[[3-(trifluoromethyl)phenyl]methylamino]pyrimidine-5-carbonitrile

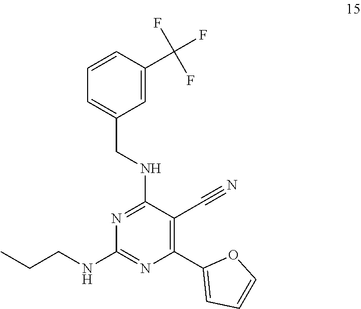 [SMILES: CCCNc1nc(NCc2cccc(C(F)(F)F)c2)c(C#N)c(-c2ccco2)n1]

The title compound was prepared in analogy to the procedure described for the preparation of Example 12, by using propan-1-amine instead of 2-methoxyethan-1-amine. The product was purified by preparative HPLC to afford Example 15 (16 mg, 33.3%) as a white powder. MS obsd. (ESI⁺) [(M+H)⁺]: 402. ¹H NMR (400 MHz, DMSO-d₆) δ ppm: 8.15 (s, 1H), 7.94 (d, J=1.0 Hz, 1H), 7.76 (s, 1H), 7.72-7.62 (m, 2H), 7.61-7.49 (m, 2H), 7.27 (d, J=3.1 Hz, 1H), 6.75-6.68 (m, 1H), 4.63 (br d, J=6.0 Hz, 2H), 3.10 (br d, J=7.1 Hz, 2H), 1.35 (d, J=7.2 Hz, 2H), 0.74 (t, J=7.4 Hz, 3H).

### Example 16

### 2-(dimethylamino)-4-(2-furyl)-6-[[3-(trifluoromethyl)phenyl]methylamino]pyrimidine-5-carbonitrile

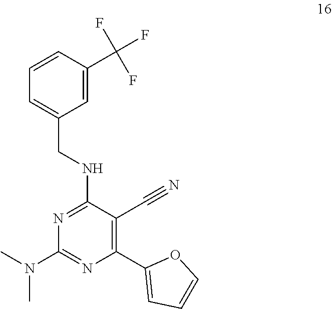 [SMILES: CN(C)c1nc(NCc2cccc(C(F)(F)F)c2)c(C#N)c(-c2ccco2)n1]

The title compound was prepared in analogy to the procedure described for the preparation of Example 12, by using dimethylamine instead of 2-methoxyethan-1-amine. The product was purified by preparative HPLC to afford Example 16 (15.8 mg, 34.2%) as an off-white powder. MS obsd. (ESI⁺) [(M+H)⁺]: 388. ¹H NMR (400 MHz, DMSO-d₆) δ ppm: 8.18-8.10 (m, 1H), 7.98-7.95 (m, 1H), 7.75 (s, 1H), 7.71-7.64 (m, 1H), 7.62-7.52 (m, 2H), 7.34 (dd, J=0.7, 3.5 Hz, 1H), 6.75-6.69 (m, 1H), 4.63 (d, J=5.9 Hz, 2H), 3.15 (br s, 3H), 3.09-2.98 (m, 3H).

### Example 17

### 4-(2-furyl)-2-methoxy-6-[[3-(trifluoromethyl)phenyl]methylamino]pyrimidine-5-carbonitrile

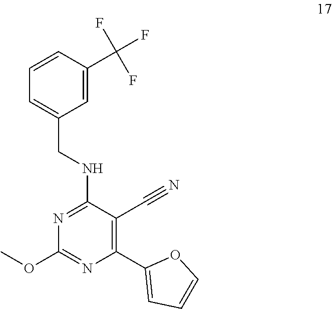 [SMILES: COc1nc(NCc2cccc(C(F)(F)F)c2)c(C#N)c(-c2ccco2)n1]

The title compound was prepared in analogy to the procedure described for the preparation of Example 12, by using methanol and sodium hydride instead of 2-methoxyethan-1-amine. The product was purified by preparative HPLC to afford Example 17 (10 mg, 44.7%) as a white powder. MS obsd. (ESI⁺) [(M+H)⁺]: 375. ¹H NMR (400 MHz, DMSO-d₆) δ ppm: 8.67 (s, 1H), 8.05 (d, J=0.9 Hz, 1H), 7.75 (s, 1H), 7.68 (d, J=7.6 Hz, 1H), 7.64-7.55 (m, 2H), 7.43 (d, J=3.1 Hz, 1H), 6.77 (dd, J=1.7, 3.5 Hz, 1H), 4.69 (d, J=6.0 Hz, 2H), 3.85 (s, 3H).

### Example 18

### 2-ethoxy-4-(2-furyl)-6-[[3-(trifluoromethyl)phenyl]methylamino]pyrimidine-5-carbonitrile

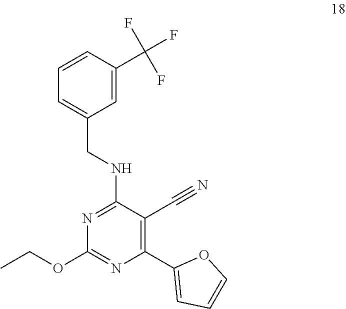 [SMILES: CCOc1nc(NCc2cccc(C(F)(F)F)c2)c(C#N)c(-c2ccco2)n1]

The title compound was prepared in analogy to the procedure described for the preparation of Example 12, by using ethanol and sodium hydride instead of 2-methoxyethan-1-amine. The product was purified by preparative HPLC to afford Example 18 (11 mg, 23.4%) as a light yellow powder. MS obsd. (ESI⁺) [(M+H)⁺]: 389. ¹H NMR (400 MHz, DMSO-d₆) δ ppm: 8.65 (s, 1H), 8.05 (d, J=1.0 Hz, 1H), 7.74 (s, 1H), 7.69-7.53 (m, 3H), 7.42 (d, J=3.5 Hz, 1H), 6.80-6.73 (m, 1H), 4.68 (d, J=5.9 Hz, 2H), 4.28 (d, J=7.1 Hz, 2H), 1.22 (t, J=7.1 Hz, 3H).

### Example 19

### 2-butoxy-4-(2-furyl)-6-[[3-(trifluoromethyl)phenyl]methylamino]pyrimidine-5-carbonitrile

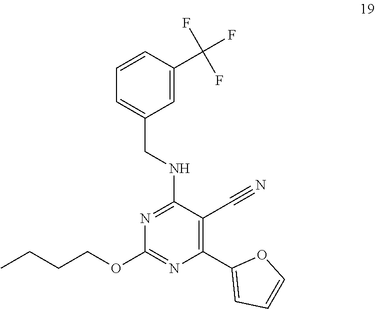 [SMILES: CCCCOc1nc(NCc2cccc(C(F)(F)F)c2)c(C#N)c(-c2ccco2)n1]

The title compound was prepared in analogy to the procedure described for the preparation of Example 12, by using butan-1-ol and sodium hydride instead of 2-methoxyethan-1-amine. The product was purified by preparative HPLC to afford Example 19 (7.6 mg, 30.2%) as an off-white powder. MS obsd. (ESI⁺) [(M+H)⁺]: 417. ¹H NMR (400 MHz, DMSO-d₆) δ ppm: 8.65 (t, J=6.0 Hz, 1H), 8.05 (dd, J=0.7, 1.7 Hz, 1H), 7.73 (s, 1H), 7.67-7.54 (m, 3H), 7.42 (dd, J=0.6, 3.5 Hz, 1H), 6.77 (dd, J=1.7, 3.5 Hz, 1H), 4.69 (d, J=5.9 Hz, 2H), 4.22 (t, J=6.6 Hz, 2H), 1.63-1.53 (m, 2H), 1.36-1.23 (m, 2H), 0.86 (t, J=7.4 Hz, 3H).

### Example 20

### 4-[(6-bromo-2,3-dimethoxy-phenyl)methylamino]-6-(2-furyl)-2-methyl-pyrimidine-5-carbonitrile

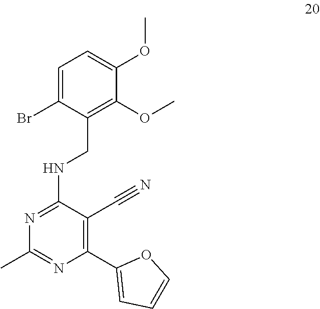

The title compound was prepared in analogy to the procedure described for the preparation of Example 1, by using 4-chloro-6-(2-furyl)-2-methyl-pyrimidine-5-carbonitrile (Int-5) and (6-bromo-2,3-dimethoxyphenyl)methanamine hydrochloride instead of 2-amino-4-chloro-6-(furan-2-yl)pyrimidine-5-carbonitrile and 1-phenylethanamine. The product was purified by preparative HPLC to afford Example 20 (2.7 mg, 6.9%). MS obsd. (ESI⁺) [(M+H)⁺]: 429. ¹H NMR (400 MHz, DMSO-d₆) δ ppm: 8.04 (d, J=1.0 Hz, 1H), 7.83 (t, J=4.8 Hz, 1H), 7.43 (d, J=3.5 Hz, 1H), 7.35 (d, J=8.8 Hz, 1H), 7.02 (d, J=8.9 Hz, 1H), 6.77 (dd, J=3.6, 1.8 Hz, 1H), 4.77 (d, J=4.9 Hz, 2H), 3.82 (s, 3H), 3.78 (s, 3H), 2.49 (s, 3H).

### Example 21

### 4-[(3-bromo-4-isopropoxy-phenyl)methylamino]-6-(2-furyl)-2-methyl-pyrimidine-5-carbonitrile

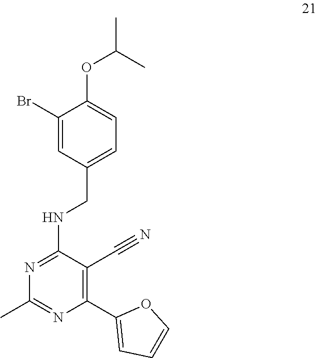 [SMILES: Cc1nc(NCc2ccc(OC(C)C)c(Br)c2)c(C#N)c(-c2ccco2)n1]

The title compound was prepared in analogy to the procedure described for the preparation of Example 1, by using 4-chloro-6-(2-furyl)-2-methyl-pyrimidine-5-carbonitrile (Int-5) and (1-phenylcyclopentyl)methanamine instead of 2-amino-4-chloro-6-(furan-2-yl)pyrimidine-5-carbonitrile and 1-phenylethanamine. The product was purified by preparative HPLC to afford Example 21 (7.3 mg, 18%). MS obsd. (ESI⁺) [(M+H)⁺]: 427. ¹H NMR (400 MHz, DMSO-d₆) δ ppm: 8.41 (t, J=6.1 Hz, 1H), 8.05 (dd, J=1.7, 0.8 Hz, 1H), 7.59 (d, J=2.2 Hz, 1H), 7.43 (dd, J=3.5, 0.7 Hz, 1H), 7.31 (dd, J=8.5, 2.1 Hz, 1H), 7.08 (d, J=8.7 Hz, 1H), 6.77 (dd, J=3.5, 1.7 Hz, 1H), 4.58-4.66 (m, 1H), 4.56 (d, J=6.0 Hz, 2H), 2.45 (s, 3H), 1.28 (s, 3H), 1.26 (s, 3H).

### Example 22

### 4-(2-furyl)-2-methyl-6-[[1-(2-methylsulfonylphenyl)cyclopropyl]amino]pyrimidine-5-carbonitrile

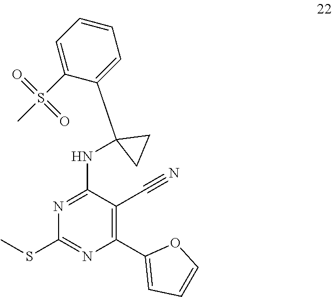 [SMILES: CSc1nc(NC2(c3ccccc3S(C)(=O)=O)CC2)c(C#N)c(-c2ccco2)n1]

The title compound was prepared in analogy to the procedure described for the preparation of Example 1, by using 4-chloro-6-(2-furyl)-2-methyl-pyrimidine-5-carbonitrile (Int-5) and (1-phenylcyclopentyl)methanamine instead of 2-amino-4-chloro-6-(furan-2-yl)pyrimidine-5-carbonitrile and 1-phenylethanamine. The product was purified by preparative HPLC to afford Example 22 (8.4 mg, 23%). MS obsd. (ESI⁺) [(M+H)⁺]: 395. ¹H NMR (400 MHz, DMSO-d₆) δ ppm: 8.08 (dd, J=7.8, 1.2 Hz, 1H), 8.03 (dd, J=1.7, 0.7 Hz, 1H), 7.97 (dd, J=7.9, 1.2 Hz, 1H), 7.70 (td, J=7.6, 1.3 Hz, 1H), 7.57 (dd, J=7.7, 1.2 Hz, 1H), 7.55 (s, 1H), 7.38 (dd, J=3.5, 0.7 Hz, 1H), 6.74 (dd, J=3.6, 1.8 Hz, 1H), 3.37 (s, 3H), 2.52-2.54 (m, 3H), 1.49 (br s, 2H), 1.37 (br s, 2H).

### Example 23

### 4-(benzylamino)-6-(2-furyl)-2-methyl-pyrimidine-5-carbonitrile

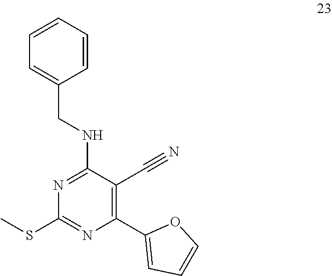 [SMILES: CSc1nc(NCc2ccccc2)c(C#N)c(-c2ccco2)n1]

The title compound was prepared in analogy to the procedure described for the preparation of Example 1, by using 4-chloro-6-(2-furyl)-2-methyl-pyrimidine-5-carbonitrile (Int-5) and phenylmethanamine instead of 2-amino-4-chloro-6-(furan-2-yl)pyrimidine-5-carbonitrile and 1-phenylethanamine. The product was purified by preparative HPLC to afford Example 23 (9 mg, 34%). MS obsd. (ESI⁺) [(M+H)⁺]: 291. ¹H NMR (400 MHz, DMSO-d₆) δ ppm: 8.44 (t, J=6.1 Hz, 1H), 8.05 (dd, J=1.6, 0.7 Hz, 1H), 7.43 (dd, J=3.5, 0.6 Hz, 1H), 7.28-7.37 (m, 4H), 7.22-7.28 (m, 1H), 6.77 (dd, J=3.5, 1.7 Hz, 1H), 4.66 (d, J=6.0 Hz, 2H), 2.43 (s, 3H).

### Example 24

### 4-[(2-chlorophenyl)methylamino]-6-(2-furyl)pyrimidine-5-carbonitrile

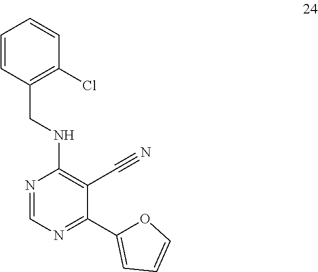 [SMILES: N#Cc1c(NCc2ccccc2Cl)ncnc1-c1ccco1]

### Step 1: Preparation of 4-chloro-6-[(2-chlorophenyl)methylamino]pyrimidine-5-carbonitrile

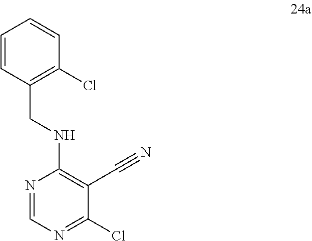 [SMILES: N#Cc1c(Cl)ncnc1NCc1ccccc1Cl]

To a mixture of 4,6-dichloropyrimidine-5-carbonitrile (100 mg, 0.57 mmol) in DCM (1 mL) was added 2-chlorobenzylamine (0.07 mL, 0.57 mmol) and triethylamine (0.16 mL, 1.15 mmol. After being stirred at 20° C. for 2 hrs. The resulting mixture was concentrated to dryness. The residue was purified by column (eluting with EtOAc/PE=10%) to afford 4-chloro-6-[(2-chlorophenyl)methylamino]pyrimidine-5-carbonitrile (Compound 24a, 100 mg) as a white solid.

### Step 2: Preparation of 4-[(2-chlorophenyl)methylamino]-6-(2-furyl)pyrimidine-5-carbonitrile

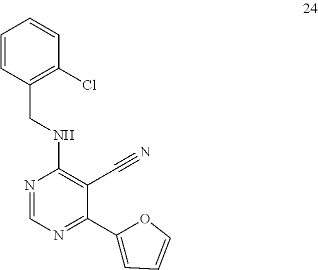 [SMILES: N#Cc1c(NCc2ccccc2Cl)ncnc1-c1ccco1]

The title compound was prepared in analogy to the procedure described for the preparation of Int-2, by using 4-chloro-6-[(2-chlorophenyl)methylamino]pyrimidine-5-carbonitrile (Compound 24a) instead of 4,6-dichloropyrimidine-5-carbonitrile. The product was purified by preparative HPLC to afford Example 24 (30 mg, 26.7%) as a white solid. MS obsd. (ESI⁺) [(M+H)⁺]: 311. ¹H NMR (400 MHz, CDCl₃) S ppm: 8.67 (s, 1H) 7.72 (d, J=0.98 Hz, 1H), 7.53 (d, J=3.67 Hz, 1H), 7.38-7.48 (m, 2H), 7.28-7.32 (m, 2H), 6.63 (dd, J=3.67, 1.71 Hz, 1H), 6.18 (s, 1H), 4.90 (d, J=5.99 Hz, 2H).

### Example 25

### 4-(benzylamino)-6-(2-furyl)pyrimidine-5-carbonitrile

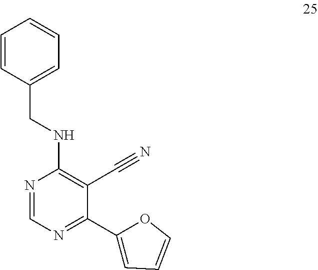 [SMILES: N#Cc1c(NCc2ccccc2)ncnc1-c1ccco1]

### Step 1: Preparation of 4-(benzylamino)-6-chloro-pyrimidine-5-carbonitrile

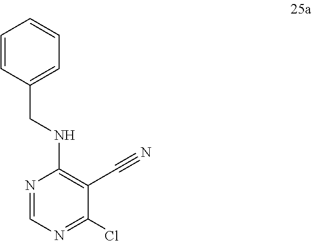 [SMILES: N#Cc1c(Cl)ncnc1NCc1ccccc1]

The title compound was prepared in analogy to the procedure described for the preparation of Compound 24a, by using benzylamine instead of 2-chlorobenzylamine. The product was purified by preparative HPLC to afford Compound 25a (85 mg, 29.9%) as a white solid.

### Step 2: Preparation of 4-(benzylamino)-6-(2-furyl)pyrimidine-5-carbonitrile

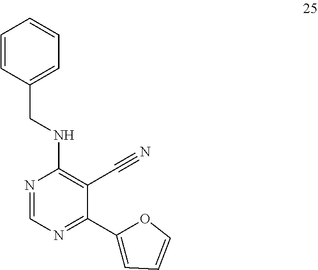 [SMILES: N#Cc1c(NCc2ccccc2)ncnc1-c1ccco1]

The title compound was prepared in analogy to the procedure described for the preparation of Example 24, by using 4-(benzylamino)-6-chloro-pyrimidine-5-carbonitrile instead of 4-chloro-6-[(2-chlorophenyl)methylamino]pyrimidine-5-carbonitrile. The product was purified by preparative HPLC to afford Example 25 (34 mg, 34.7%) as a white solid. MS obsd. (ESI⁺) [(M+H)⁺]: 277. ¹H NMR (400 MHz, DMSO-d₆) δ ppm: 8.60 (t, J=5.96 Hz, 1H), 8.56 (s, 1H), 8.08 (d, J=0.88 Hz, 1H), 7.46 (dd, J=3.51, 0.63 Hz, 1H), 7.28-7.36 (m, 4H), 7.18-7.26 (m, 1H), 6.79 (dd, J=3.51, 1.76 Hz, 1H), 4.67 (d, J=6.02 Hz, 2H).

### Example 26

### 4-(2-furyl)-6-(isobutylamino)pyrimidine-5-carbonitrile

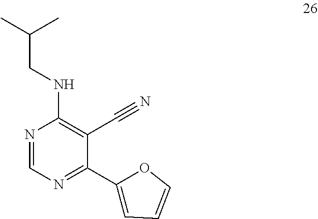 [SMILES: CC(C)CNc1ncnc(-c2ccco2)c1C#N]

### Step 1: Preparation of 4-chloro-6-(isobutylamino)pyrimidine-5-carbonitrile

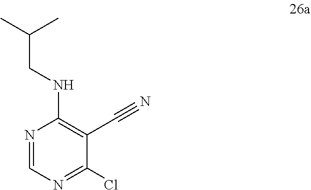

The title compound was prepared in analogy to the procedure described for the preparation of Compound 24a, by using isobutylamine instead of 2-chlorobenzylamine. The product was purified by preparative HPLC to afford Compound 26a (85 mg, 32.6%) as a white solid.

### Step 2: Preparation of 4-(2-furyl)-6-(isobutylamino)pyrimidine-5-carbonitrile

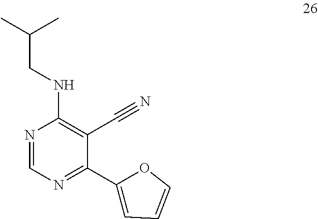 [SMILES: CC(C)CNc1ncnc(-c2ccco2)c1C#N]

The title compound was prepared in analogy to the procedure described for the preparation of Example 24, by using 4-chloro-6-(isobutylamino)pyrimidine-5-carbonitrile instead of 4-chloro-6-[(2-chlorophenyl)methylamino]pyrimidine-5-carbonitrile. The product was purified by preparative HPLC to afford Example 26 (34 mg, 33.0%) as a white solid. MS obsd. (ESI⁺) [(M+H)⁺]: 243. ¹H NMR (400 MHz, DMSO-d₆) δ ppm: 8.56 (s, 1H), 7.98-8.11 (m, 2H), 7.44 (dd, J=3.51, 0.63 Hz, 1H), 6.78 (dd, J=3.51, 1.76 Hz, 1H), 3.28 (dd, J=7.03, 6.15 Hz, 2H), 1.96 (dquin, J=13.59, 6.80, 6.80, 6.80, 6.80 Hz, 1H), 0.87 (d, J=6.65 Hz, 6H).

### Example 27

### 4-(cyclohexylmethylamino)-6-(2-furyl)pyrimidine-5-carbonitrile

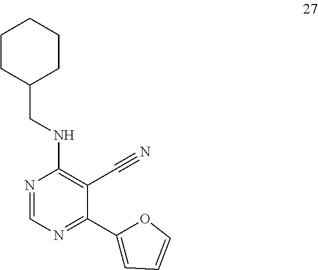 [SMILES: N#Cc1c(NCC2CCCCC2)ncnc1-c1ccco1]

### Step 1: Preparation of 4-chloro-6-(cyclohexylmethylamino)pyrimidine-5-carbonitrile

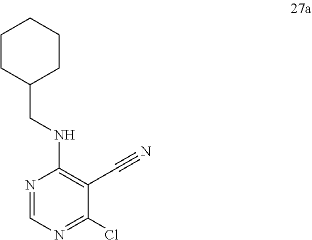 [SMILES: N#Cc1c(Cl)ncnc1NCC1CCCCC1]

The title compound was prepared in analogy to the procedure described for the preparation of Compound 24a, by using cyclohexanemethylamine instead of 2-chlorobenzylamine. The product was purified by preparative HPLC to afford Compound 27a (90 mg, 29.4%) as a white solid.

### Step 2: Preparation of 4-(cyclohexylmethylamino)-6-(2-furyl)pyrimidine-5-carbonitrile

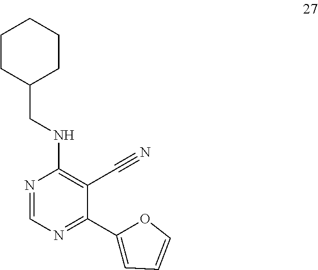 [SMILES: N#Cc1c(NCC2CCCCC2)ncnc1-c1ccco1]

The title compound was prepared in analogy to the procedure described for the preparation of Example 24, by using 4-chloro-6-(cyclohexylmethylamino)pyrimidine-5-carbonitrile (Compound 27a) instead of 4-chloro-6-[(2-chlorophenyl)methylamino]pyrimidine-5-carbonitrile (Compound 24a). The product was purified by preparative HPLC to afford Example 27 (43 mg, 41.2%) as a white solid. MS obsd. (ESI⁺) [(M+H)⁺]: 283. ¹H NMR (400 MHz, DMSO-d₆) δ ppm: 8.56 (s, 1H), 8.03-8.09 (m, 1H), 8.01 (t, J=5.71 Hz, 1H), 7.44 (dd, J=3.51, 0.63 Hz, 1H), 6.78 (dd, J=3.51, 1.76 Hz, 1H), 3.27-3.31 (m, 2H), 1.57-1.74 (m, 6H), 1.07-1.25 (m, 3H), 0.83-1.01 (m, 2H).

### Example 28

### 4-(2-furyl)-6-(2-phenylethylamino)pyrimidine-5-carbonitrile

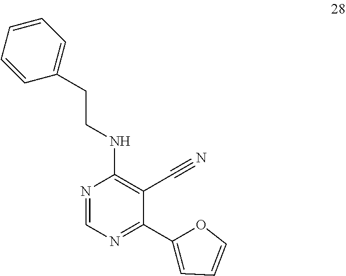 [SMILES: N#Cc1c(NCCc2ccccc2)ncnc1-c1ccco1]

### Step 1: Preparation of 4-chloro-6-(2-phenylethylamino)pyrimidine-5-carbonitrile

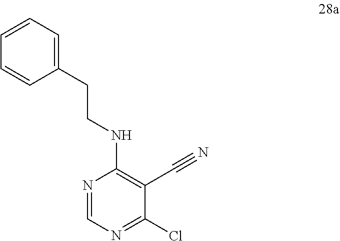 [SMILES: N#Cc1c(Cl)ncnc1NCCc1ccccc1]

The title compound was prepared in analogy to the procedure described for the preparation of Compound 24a, by using phenethylamine instead of 2-chlorobenzylamine. The product was purified by preparative HPLC to afford Compound 28a (120 mg, 39.95%) as a white solid. MS obsd. (ESI⁺) [(M+H)⁺]: 259. ¹H NMR (400 MHz, CDCl₃) δ ppm: 8.49 (s, 1H), 7.32-7.39 (m, 2H), 7.25-7.30 (m, 1H), 7.18-7.25 (m, 2H), 5.77 (br s, 1H), 3.80-3.90 (m, 2H), 2.95 (t, J=7.09 Hz, 2H).

### Step 2: Preparation of 4-(2-furyl)-6-(2-phenylethylamino)pyrimidine-5-carbonitrile

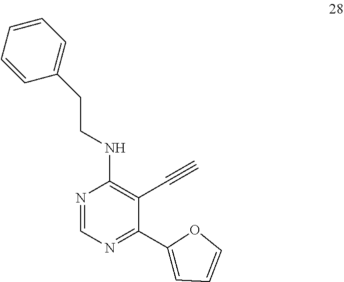 [SMILES: C#Cc1c(NCCc2ccccc2)ncnc1-c1ccco1]

The title compound was prepared in analogy to the procedure described for the preparation of Example 24, by using 4-chloro-6-(2-phenylethylamino)pyrimidine-5-carbonitrile (Compound 28a) instead of 4-chloro-6-[(2-chlorophenyl)methylamino]pyrimidine-5-carbonitrile (Compound 24a). The product was purified by preparative HPLC to afford Example 28 (35.4 mg, 31.6%) as a white solid. MS obsd. (ESI⁺) [(M+H)⁺]: 291. ¹H NMR (400 MHz, CD₃OD) δ ppm: 8.52 (s, 1H), 7.85 (d, J=0.98 Hz, 1H), 7.52 (d, J=3.55 Hz, 1H), 7.15-7.33 (m, 5H), 6.71 (dd, J=3.61, 1.77 Hz, 1H), 3.73-3.84 (m, 2H), 2.88-3.00 (m, 2H).

### Example 29

### 4-(cyclopentylmethylamino)-6-(2-furyl)pyrimidine-5-carbonitrile

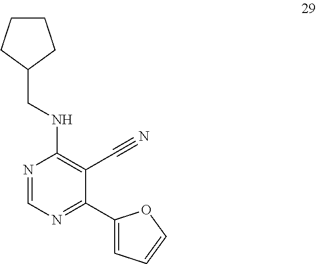 [SMILES: N#Cc1c(NCC2CCCC2)ncnc1-c1ccco1]

### Step 1: Preparation of 4-chloro-6-(cyclopentylmethylamino)pyrimidine-5-carbonitrile

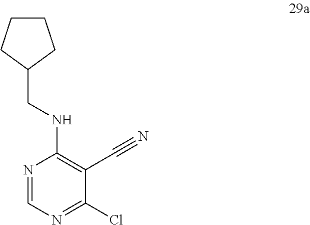 [SMILES: N#Cc1c(Cl)ncnc1NCC1CCCC1]

The title compound was prepared in analogy to the procedure described for the preparation of Compound 24a, by using aminomethylcyclopentane hydrochloride instead of 2-chlorobenzylamine. The product was purified by preparative HPLC to afford Compound 29a (100 mg, 36.8%) as a white solid.

### Step 2: Preparation of 4-(cyclopentylmethylamino)-6-(2-furyl)pyrimidine-5-carbonitrile

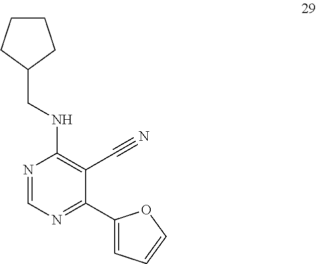 [SMILES: N#Cc1c(NCC2CCCC2)ncnc1-c1ccco1]

The title compound was prepared in analogy to the procedure described for the preparation of Example 24, by using 4-chloro-6-(cyclopentylmethylamino)pyrimidine-5-carbonitrile (Compound 29a) instead of 4-chloro-6-[(2-chlorophenyl)methylamino]pyrimidine-5-carbonitrile (Compound 24a). The product was purified by preparative HPLC to afford Example 29 (59 mg, 49.5%) as a white solid. MS obsd. (ESI⁺) [(M+H)⁺]: 269. ¹H NMR (400 MHz, DMSO-d₆) δ ppm: 8.56 (s, 1H), 7.99-8.12 (m, 2H), 7.44 (dd, J=3.58, 0.69 Hz, 1H), 6.78 (dd, J=3.64, 1.76 Hz, 1H), 3.37 (dd, J=7.28, 6.15 Hz, 2H), 2.21-2.29 (m, 1H), 1.45-1.71 (m, 6H), 1.19-1.33 (m, 2H).

### Example 30

### 4-(2-furyl)-6-[(1-phenylcyclopentyl)methylamino]pyrimidine-5-carbonitrile

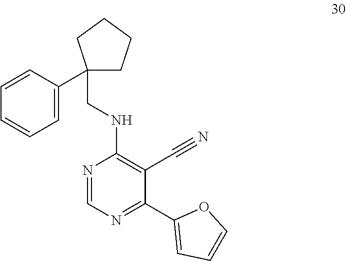 [SMILES: N#Cc1c(NCC2(c3ccccc3)CCCC2)ncnc1-c1ccco1]

The title compound was prepared in analogy to the procedure described for the preparation of Example 1, by using (1-phenylcyclopentyl)methanamine instead of phenylethanamine. The product was purified by preparative HPLC to afford Example 30 (35 mg, 25.32%) as a white solid. MS obsd. (ESI⁺)[(M+H)⁺]: 345.2. ¹H NMR (400 MHz, DMSO-d₆) δ ppm: 8.38 (s, 1H), 8.06 (d, J=0.9 Hz, 1H), 1.63 (s, 2H), 7.41 (d, J=3.5 Hz, 1H), 7.28 (dt, J=12.9, 7.5 Hz, 4H), 7.16 (d, J=7.1 Hz, 2H), 6.77 (dd, J=3.5, 1.7 Hz, 1H), 3.67 (d, J=6.3 Hz, 2H), 2.05 (s, 2H), 1.81 (s, 4H).

### Example 31

### 4-[(2-aminophenyl)methylamino]-6-(2-furyl)pyrimidine-5-carbonitrile

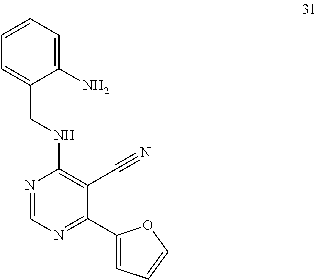 [SMILES: N#Cc1c(NCc2ccccc2N)ncnc1-c1ccco1]

The title compound was prepared in analogy to the procedure described for the preparation of Example 1, by using 4-chloro-6-(2-furyl)pyrimidine-5-carbonitrile (Int-2) and 2-aminobenzylamine instead of 2-amino-4-chloro-6-(furan-2-yl)pyrimidine-5-carbonitrile (Int-1) and 1-phenylethanamine. The product was purified by preparative HPLC to afford Example 31 (20.9 mg, 18.6%) as a white solid. MS obsd. (ESI⁺)[(M+H)⁺]: 292.1. ¹H NMR (400 MHz, DMSO-d₆) δ ppm: 8.58 (s, 1H), 8.38 (s, 1H), 8.08 (s, 1H), 7.46 (d, J=3.0 Hz, 1H), 7.08 (d, J=7.3 Hz, 1H), 6.96 (t, J=7.4 Hz, 1H), 6.79 (s, 1H), 6.64 (d, J=7.8 Hz, 1H), 6.50 (t, J=7.2 Hz, 1H), 5.22 (s, 2H), 4.49 (d, J=4.9 Hz, 2H).

### Example 32

### 6-(2-furyl)-N2,N2-dimethyl-N4-[1-[3-(trifluoromethyl)phenyl]ethyl]pyrimidine-2,4-diamine

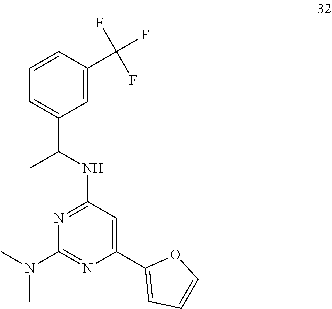 [SMILES: CC(Nc1cc(-c2ccco2)nc(N(C)C)n1)c1cccc(C(F)(F)F)c1]

The title compound was prepared in analogy to the procedure described for the preparation of Example 1, by using 4-chloro-6-(2-furyl)-N,N-dimethyl-pyrimidin-2-amine (Int-3) and 1-(3-(trifluoromethyl)phenyl)ethan-1-amine instead of 2-amino-4-chloro-6-(furan-2-yl)pyrimidine-5-carbonitrile (Int-1) and 1-phenylethanamine. The product was purified by preparative HPLC to afford Example 32 (31 mg, 77%). MS obsd. (ESI⁺) [(M+H)⁺]: 377. ¹H NMR (400 MHz, DMSO-d₆) δ ppm: 7.90 (br s, 1H), 7.54-7.75 (m, 4H), 7.20 (br s, 1H), 6.68 (br s, 1H), 6.28 (br s, 1H), 5.23 (br s, 1H), 3.05 (br s, 6H), 1.49 (d, J=7.1 Hz, 3H).

### Example 33

### N4-[(2-aminophenyl)methyl]-6-(2-furyl)-N2,N2-dimethyl-pyrimidine-2,4-diamine

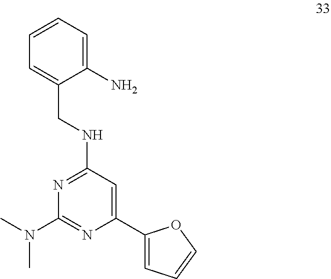 [SMILES: CN(C)c1nc(NCc2ccccc2N)cc(-c2ccco2)n1]

The title compound was prepared in analogy to the procedure described for the preparation of Example 1, by using 4-chloro-6-(2-furyl)-N,N-dimethyl-pyrimidin-2-amine (Int 3) and 2-(aminomethyl)aniline instead of 2-amino-4-chloro-6-(furan-2-yl)pyrimidine-5-carbonitrile (Int 1) and 1-phenylethanamine. The product was purified by preparative HPLC to afford Example 33 (26 mg, 47%) as a white solid. MS obsd. (ESI⁺) [(M+H)⁺]: 310. ¹H NMR (400 MHz, DMSO-d₆) δ ppm: 7.80 (s, 1H), 7.53 (br s, 1H), 7.06 (br d, J=7.3 Hz, 2H), 6.97 (t, J=7.2 Hz, 1H), 6.59-6.67 (m, 2H), 6.53 (t, J=7.3 Hz, 1H), 6.14 (br s, 1H), 5.24 (br s, 1H), 4.42 (br s, 2H), 3.04-3.14 (m, 6H).

### Example 34

### 6-(2-furyl)-2-methoxy-N-[1-[3-(trifluoromethyl)phenyl]ethyl]pyrimidin-4-amine

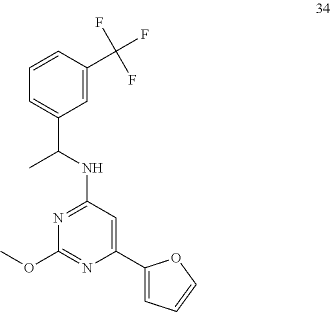 [SMILES: COc1nc(NC(C)c2cccc(C(F)(F)F)c2)cc(-c2ccco2)n1]

The title compound was prepared in analogy to the procedure described for the preparation of Example 1, by using 4-chloro-6-(2-furyl)-2-methoxy-pyrimidine (Int-4) and 1-(3-(trifluoromethyl)phenyl)ethan-1-amine instead of 2-amino-4-chloro-6-(furan-2-yl)pyrimidine-5-carbonitrile (Int-1) and 1-phenylethanamine. The product was purified by preparative HPLC to afford Example 34 (11 mg, 21%) as a white solid. MS obsd. (ESI⁺) [(M+H)⁺]: 364. ¹H NMR (400 MHz, DMSO-d₆) δ ppm: 8.22 (br s, 1H), 7.85 (s, 1H), 7.65-7.78 (m, 2H), 7.55-7.61 (m, 2H), 7.06 (br d, J=2.8 Hz, 1H), 6.64 (dd, J=3.4, 1.7 Hz, 1H), 6.54 (br s, 1H), 5.25 (br s, 1H), 3.74 (br s, 3H), 1.48 (d, J=7.1 Hz, 3H).

### Example 35

### 6-(2-furyl)-5-methoxy-N4-[[3-(trifluoromethyl)phenyl]methyl]pyrimidine-2,4-diamine

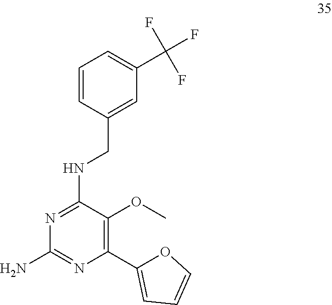 [SMILES: COc1c(NCc2cccc(C(F)(F)F)c2)nc(N)nc1-c1ccco1]

The title compound was prepared in analogy to the procedure described for the preparation of Example 1, by using 4-chloro-6-(2-furyl)-5-methoxy-pyrimidin-2-amine (Int-6) and (3-(trifluoromethyl)phenyl)methanamine instead of 2-amino-4-chloro-6-(furan-2-yl)pyrimidine-5-carbonitrile (Int-1) and 1-phenylethanamine. The product was purified by preparative HPLC to afford Example 35 (26.5 mg). MS obsd. (ESI⁺) [(M+H)⁺]: 365. ¹H NMR (400 MHz, DMSO-d₆) δ ppm: 8.05 (br s, 1H), 7.91 (s, 1H), 7.54-7.72 (m, 4H), 7.15 (br d, J=2.9 Hz, 1H), 6.68-6.76 (m, 1H), 6.34 (br s, 2H), 4.67 (d, J=6.2 Hz, 2H), 3.64 (s, 3H).

### Example 36

### N4-[(2-aminophenyl)methyl]-6-(2-thienyl)pyrimidine-2,4-diamine

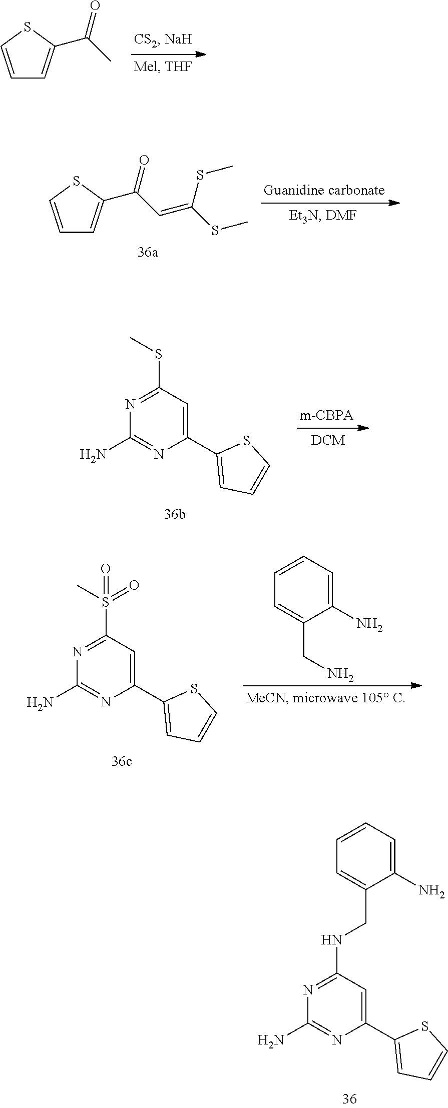

### Step 1: Preparation of 3,3-bis(methylsulfanyl)-1-(2-thienyl)prop-2-en-1-one

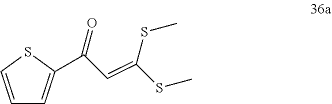

To a stirred solution of NaH (6.34 g, 159 mmol) in THF (200 ml) at 0° C. was added 1-(thiophen-2-yl)ethanone (10 g, 79.3 mmol) dropwise, the suspension was stirred for 20 min, and carbon disulfide (6.64 g, 5.41 ml, 87.2 mmol) was added dropwise. The reaction mixture was allowed to stir for 30 min, then Mel (24.7 g, 10.9 ml, 174 mmol) was added dropwise. The mixture was allowed to rt overnight. The reaction mixture was poured into ice water, extracted with EtOAc, dried over anhydrous sodium sulfate. The solvent was distilled under reduced pressure and the dithioacetal body was obtained. Recrystallization from n-hexane and EtOAc afforded Compound 36a (12.5 g, 68.5%) as a yellow solid. MS obsd. (ESI⁺) [(M+H)⁺]: 231.

### Step 2: Preparation of 4-methylsulfanyl-6-(2-thienyl)pyrimidin-2-amine

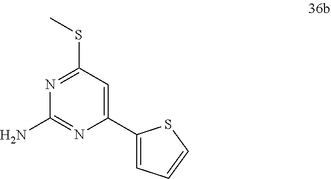

To a solution of 3,3-bis(methylthio)-1-(thiophen-2-yl)prop-2-en-1-one (Compound 36a, 1 g, 4.34 mmol) and guanidine carbonate (938 mg, 5.21 mmol) in DMF (20 ml) was added Et₃N (1.1 g, 1.51 ml, 10.9 mmol). After being heated at reflux for 2 hrs, the reaction mixture was then cooled to rt. After addition of 30 ml water, a precipitate appeared which was collected by filtration and washed with water to afford Compound 36b (0.84 g) as a yellow solid. MS obsd. (ESI⁺) [(M+H)⁺]: 224.

### Step 3: Preparation of 4-methylsulfonyl-6-(2-thienyl)pyrimidin-2-amine

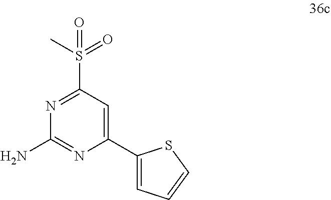 [SMILES: CS(=O)(=O)c1cc(-c2cccs2)nc(N)n1]

To a solution of 4-(methylthio)-6-(thiophen-2-yl)pyrimidin-2-amine (Compound 36b, 800 mg, 3.58 mmol) in CH₂Cl₂ (35 ml) was added 3-chlorobenzoperoxoic acid (1.82 g, 8.96 mmol). After reaction was stirred at 25° C. for 1 hr, the volume of reaction mixture was reduced in vacuo. The crude material was purified by flash chromatography (silica gel, 40 g, 0% to 40% EtOAc in hexane) to afford Compound 36c (756 mg, 82.7%) as a yellow solid. MS obsd. (ESI⁺) [(M+H)⁺]: 256.

### Step 4: Preparation of N4-[(2-aminophenyl)methyl]-6-(2-thienyl)pyrimidine-2,4-diamine

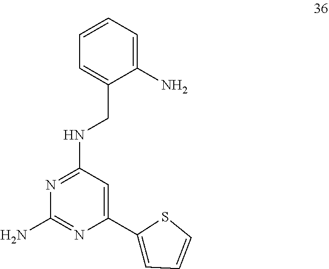 [SMILES: Nc1nc(NCc2ccccc2N)cc(-c2cccs2)n1]

To a 10 mL microwave vial was added 4-(methylsulfonyl)-6-(thiophen-2-yl)pyrimidin-2-amine (Compound 36c, 200 mg), 2-(aminomethyl)aniline (239 mg, 1.96 mmol) in MeCN (10 mL). The vial was capped and heated in the microwave at 105° C. for 30 min. The crude reaction mixture was concentrated in vacuo. The crude material was purified by preparative HPLC to afford Example 36 (81 mg, 34.1%) as a white powder. MS obsd. (ESI⁺) [(M+H)⁺]: 298. ¹H NMR (400 MHz, DMSO-d₆) δ ppm: 7.67-7.46 (m, 2H), 7.36-7.17 (m, 1H), 7.15-7.07 (m, 1H), 7.07-6.98 (m, 1H), 6.98-6.88 (m, 1H), 6.66-6.57 (m, 1H), 6.54-6.45 (m, 1H), 6.20 (s, 1H), 6.16-6.01 (m, 2H), 5.16-5.02 (m, 2H), 4.44-4.24 (m, 2H).

### Example 37

### Step 1: Preparation of ethyl 2-amino-4-[(4-chlorophenyl)methylamino]-6-(2-furyl)pyrimidine-5-carboxylate

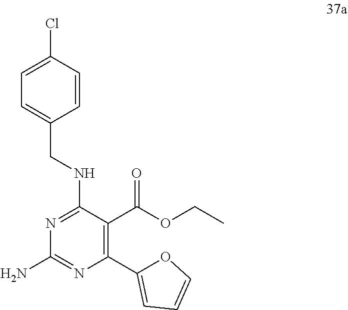 [SMILES: CCOC(=O)c1c(NCc2ccc(Cl)cc2)nc(N)nc1-c1ccco1]

To a 10 mL microwave vial was added ethyl 2-amino-4-(furan-2-yl)-6-(methylsulfonyl)pyrimidine-5-carboxylate (Int-10, 150 mg), (4-chlorophenyl)methanamine (102 mg) and DIPEA (187 mg, 252 μL, 1.45 mmol) in MeCN (5 ml). The vial was capped and heated in the microwave at 105° C. for 30 min. The crude reaction mixture was concentrated in vacuo. The crude material was purified by flash chromatography (eluting with EtOAc/PE=0 to 50%) to Compound 37a (150 mg, 83.5%) as an off-white solid. MS obsd. (ESI⁺) [(M+H)⁺]: 373.

### Step 2: Preparation of 2-amino-4-[(4-chlorophenyl)methylamino]-6-(2-furyl)pyrimidine-5-carboxylic Acid

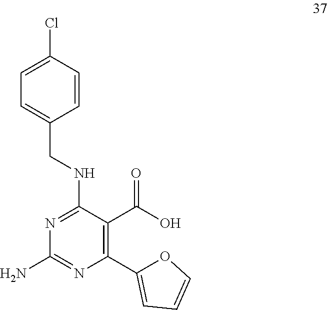 [SMILES: Nc1nc(NCc2ccc(Cl)cc2)c(C(=O)O)c(-c2ccco2)n1]

A mixture of ethyl 2-amino-4-((4-chlorobenzyl)amino)-6-(furan-2-yl)pyrimidine-5-carboxylate (compound 37a, 150 mg) and KOH (113 mg, 2.01 mmol) in dioxane (5 mL)/H₂O (3 mL) was stirred at 90° C. overnight. The reaction mixture was acidified to pH=8 with 1 M HCl. After filtration, the filtrate was purified by prep-HPLC to afford Example 37 (45 mg, 32.1%) as a white solid. MS obsd. (ESI⁺) [(M+H)⁺]: 345. ¹H NMR (400 MHz, DMSO-d₆) δ ppm: 8.31-8.15 (m, 1H), 7.72 (dd, J=0.8, 1.7 Hz, 1H), 7.41-7.32 (m, 4H), 6.78 (dd, J=0.8, 3.4 Hz, 1H), 6.62-6.51 (m, 3H), 4.59 (d, J=6.1 Hz, 2H).

### Example 38

### Step 1: Preparation of ethyl 2-amino-4-(2-furyl)-6-[[3-(trifluoromethyl)phenyl]methylamino]pyrimidine-5-carboxylate

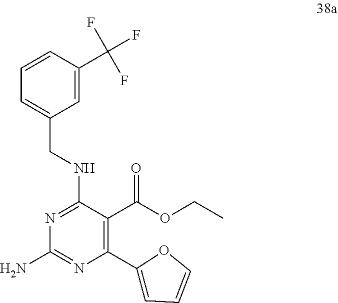 [SMILES: CCOC(=O)c1c(NCc2cccc(C(F)(F)F)c2)nc(N)nc1-c1ccco1]

The title compound was prepared in analogy to the procedure described for the preparation of Compound 37a, by using (3-(trifluoromethyl)phenyl)methanamine instead of (4-chlorophenyl)methanamine. The product was purified by preparative HPLC to afford Compound 38a (12 mg, 18%) as an off-white powder. MS obsd. (ESI⁺) [(M+H)⁺]: 407.

### Step 2: Preparation of 2-amino-4-(2-furyl)-6-[[3-(trifluoromethyl)phenyl]methylamino]pyrimidine-5-carboxylic Acid

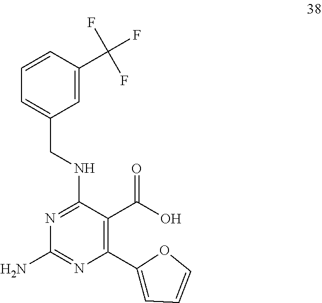 [SMILES: Nc1nc(NCc2cccc(C(F)(F)F)c2)c(C(=O)O)c(-c2ccco2)n1]

The title compound was prepared in analogy to the procedure described for the preparation of Example 37, by using ethyl 2-amino-4-(furan-2-yl)-6-((3-(trifluoromethyl)benzyl)amino)pyrimidine-5-carboxylate (Compound 38a) instead of ethyl 2-amino-4-((4-chlorobenzyl)amino)-6-(furan-2-yl)pyrimidine-5-carboxylate (Compound 37a). The product was purified by preparative HPLC to afford Example 38 (7 mg, 14.9%) as a yellow solid. MS obsd. (ESI⁺) [(M+H)⁺]: 379. ¹H NMR (400 MHz, DMSO-d₆) δ ppm: 8.76-8.43 (m, 1H), 7.92 (br s, 1H), 7.74-7.66 (m, 2H), 7.65-7.54 (m, 2H), 7.06-6.97 (m, 1H), 6.69 (br s, 1H), 4.73 (d, J=6.0 Hz, 2H).

### Example 39

### 2-amino-4-(2-furyl)-6-(o-tolylmethylamino)pyrimidine-5-carboxylic Acid

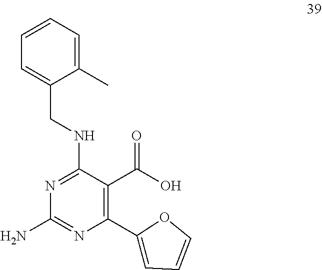 [SMILES: Cc1ccccc1CNc1nc(N)nc(-c2ccco2)c1C(=O)O]

### Step 1: Preparation of ethyl 2-amino-4-(2-furyl)-6-(o-tolylmethylamino)pyrimidine-5-carboxylate

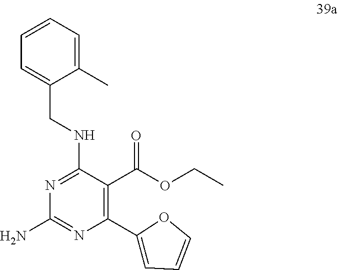 [SMILES: CCOC(=O)c1c(NCc2ccccc2C)nc(N)nc1-c1ccco1]

The title compound was prepared in analogy to the procedure described for the preparation Compound 37a, by using o-tolylmethanamine instead of (4-chlorophenyl)methanamine. The product was purified by preparative HPLC to afford Compound 39a, (115 mg, 67.7%) as an off-white solid. MS obsd. (ESI⁺) [(M+H)⁺]: 353.

### Step 2: Preparation of 2-amino-4-(2-furyl)-6-(o-tolylmethylamino)pyrimidine-5-carboxylic Acid

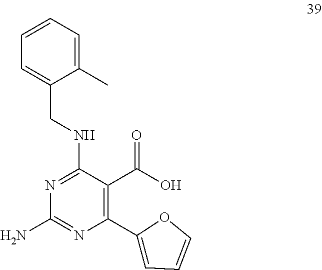 [SMILES: Cc1ccccc1CNc1nc(N)nc(-c2ccco2)c1C(=O)O]

The title compound was prepared in analogy to the procedure described for the preparation of Example 37, by using ethyl 2-amino-4-(furan-2-yl)-6-((2-methylbenzyl)amino)pyrimidine-5-carboxylate instead of ethyl 2-amino-4-((4-chlorobenzyl)amino)-6-(furan-2-yl)pyrimidine-5-carboxylate (Compound 37a). The product was purified by preparative HPLC to afford Example 39 (27 mg, 29%) as an off-white solid. MS obsd. (ESI⁺) [(M+H)⁺]: 325. ¹H NMR (400 MHz, DMSO-d₆) δ ppm: 8.32-8.09 (m, 1H), 7.73-7.69 (m, 1H), 7.26-7.12 (m, 4H), 6.77 (dd, J=0.7, 3.4 Hz, 1H), 6.53 (br dd, J=1.7, 3.3 Hz, 3H), 4.57 (d, J=5.6 Hz, 2H), 2.30 (s, 3H).

### Example 40

### 2-amino-4-(2-furyl)-6-[(2-methoxyphenyl)methylamino]pyrimidine-5-carboxylic Acid

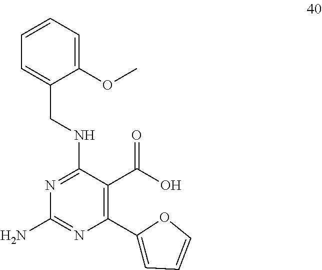 [SMILES: COc1ccccc1CNc1nc(N)nc(-c2ccco2)c1C(=O)O]

### Step 1: Preparation of ethyl 2-amino-4-(2-furyl)-6-[(2-methoxyphenyl)methylamino]pyrimidine-5-carboxylate

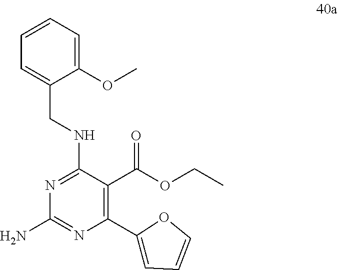 [SMILES: CCOC(=O)c1c(NCc2ccccc2OC)nc(N)nc1-c1ccco1]

The title compound was prepared in analogy to the procedure described for the preparation of Example 37, by using (2-methoxyphenyl)methanamine instead of (4-chlorophenyl)methanamine. The product was purified by preparative HPLC to afford Compound 40a (50 mg, 84.5%) as a yellow solid. MS obsd. (ESI⁺) [(M+H)⁺]: 369.

### Step 2: Preparation of 2-amino-4-(2-furyl)-6-[(2-methoxyphenyl)methylamino]pyrimidine-5-carboxylic Acid

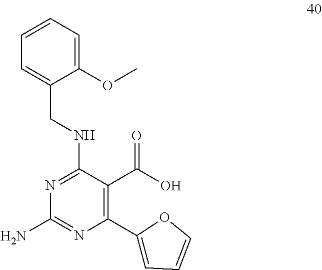 [SMILES: COc1ccccc1CNc1nc(N)nc(-c2ccco2)c1C(=O)O]

The title compound was prepared in analogy to the procedure described for the preparation of Example 37, by using ethyl 2-amino-4-(furan-2-yl)-6-((2-methoxybenzyl)amino)pyrimidine-5-carboxylate (Compound 40a) instead of ethyl 2-amino-4-((4-chlorobenzyl)amino)-6-(furan-2-yl)pyrimidine-5-carboxylate (Compound 37a). The product was purified by preparative HPLC to afford Example 40 (12 mg, 32.1%) as a white powder. MS obsd. (ESI⁺) [(M+H)⁺]: 341. ¹H NMR (400 MHz, DMSO-d₆) δ ppm: 8.74-8.56 (m, 1H), 7.98 (s, 1H), 7.33-7.23 (m, 1H), 7.22-7.14 (m, 1H), 7.09 (br s, 1H), 7.02 (d, J=7.7 Hz, 1H), 6.95-6.90 (m, 1H), 6.73 (dd, J=1.7, 3.4 Hz, 1H), 4.64 (d, J=5.9 Hz, 3H), 3.84 (s, 7H).

### Example 41

### 2-amino-4-(cyclopentylmethylamino)-6-(2-furyl)pyrimidine-5-carboxylic acid

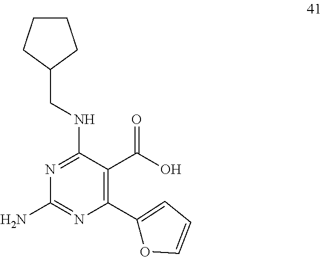 [SMILES: Nc1nc(NCC2CCCC2)c(C(=O)O)c(-c2ccco2)n1]

### Step 1: Preparation of 2-amino-4-(cyclopentylmethylamino)-6-(2-furyl)pyrimidine-5-carboxylate

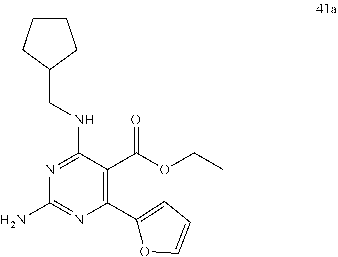 [SMILES: CCOC(=O)c1c(NCC2CCCC2)nc(N)nc1-c1ccco1]

The title compound was prepared in analogy to the procedure described for the preparation of Compound 37a, by using cyclopentylmethanamine instead of (4-chlorophenyl)methanamine. The product was purified by preparative HPLC to afford Compound 41a (100 mg, 94.23%) as a white solid. MS obsd. (ESI⁺) [(M+H)⁺]: 331.2.

### Step 2. Preparation of 2-amino-4-(cyclopentylmethylamino)-6-(2-furyl)pyrimidine-5-carboxylic Acid

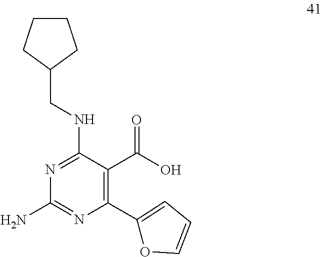 [SMILES: Nc1nc(NCC2CCCC2)c(C(=O)O)c(-c2ccco2)n1]

The title compound was prepared in analogy to the procedure described for the preparation of Example 37, by using ethyl 2-amino-4-(cyclopentylmethylamino)-6-(2-furyl)pyrimidine-5-carboxylate (Compound 41a) instead of ethyl 2-amino-4-((4-chlorobenzyl)amino)-6-(furan-2-yl)pyrimidine-5-carboxylate (Compound 37a). The product was purified by preparative HPLC to afford Example 41 (39 mg, 84.5% yield) as a white solid. MS obsd. (ESI⁺)[(M+H)⁺]: 303.1. ¹H NMR (400 MHz, DMSO-d₆) δ ppm: 8.39 (s, 1H), 7.61 (s, 1H), 6.74 (d, J=3.3 Hz, 1H), 6.46 (dd, J=3.3 Hz, 1.7 Hz, 1H), 6.12 (s, 2H), 3.26 (dd, J=6.9 Hz, 5.7 Hz, 2H), 2.03-2.15 (m, 1H), 1.66-1.76 (m, 2H), 1.47-1.64 (m, 4H), 1.22 (dd, J=12.2, 6.9 Hz, 2H).

### Example 42

### 2-amino-4-[benzyl(methyl)amino]-6-(2-furyl)pyrimidine-5-carboxylic Acid

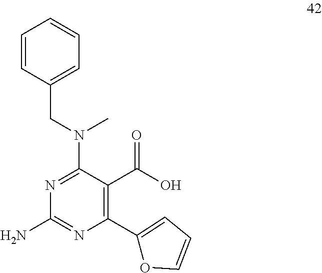 [SMILES: CN(Cc1ccccc1)c1nc(N)nc(-c2ccco2)c1C(=O)O]

### Step 1: Preparation of ethyl 2-amino-4-[benzyl(methyl)amino]-6-(2-furyl)pyrimidine-5-carboxylate

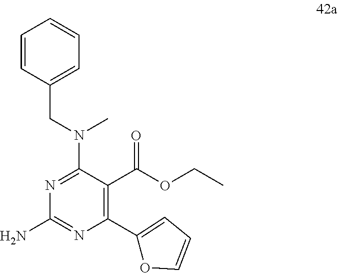 [SMILES: CCOC(=O)c1c(-c2ccco2)nc(N)nc1N(C)Cc1ccccc1]

The title compound was prepared in analogy to the procedure described for the preparation of Compound 37a, by using N-methylaniline instead of (4-chlorophenyl)methanamine. The product was purified by preparative HPLC to afford Compound 42a as a white solid. MS obsd. (ESI⁺)[(M+H)⁺]: 353.2.

### Step 2. Preparation of 2-amino-4-[benzyl(methyl)amino]-6-(2-furyl)pyrimidine-5-carboxylic Acid

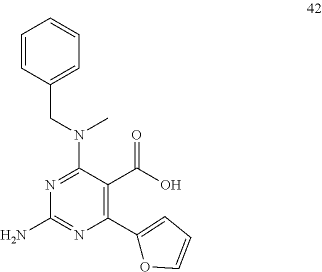 [SMILES: CN(Cc1ccccc1)c1nc(N)nc(-c2ccco2)c1C(=O)O]

The title compound was prepared in analogy to the procedure described for the preparation of Example 37, by using ethyl 2-amino-4-[benzyl(methyl)amino]-6-(2-furyl)pyrimidine-5-carboxylate (Compound 42a) instead of ethyl 2-amino-4-((4-chlorobenzyl)amino)-6-(furan-2-yl)pyrimidine-5-carboxylate (Compound 37a). The product was purified by preparative HPLC to afford Example 42 (5.8 mg) as a white solid. MS obsd. (ESI⁺)[(M+H)⁺]: 325.1. ¹H NMR (400 MHz, DMSO-d₆) δ ppm: 7.75 (s, 1H), 7.30 (m, 5H), 7.00 (s, 1H), 6.58 (s, 1H), 6.29 (s, 2H), 4.85 (s, 2H), 2.97 (s, 3H).

### Example 43

### 4-(cyclopentylmethylamino)-2-(dimethylamino)-6-(2-furyl)pyrimidine-5-carboxylic Acid

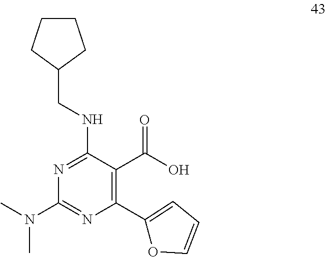 [SMILES: CN(C)c1nc(NCC2CCCC2)c(C(=O)O)c(-c2ccco2)n1]

### Step 1: Preparation of ethyl 2-(dimethylamino)-4-(2-furyl)-6-methylsulfonyl-pyrimidine-5-carboxylate

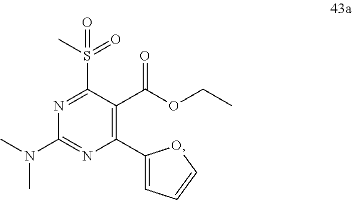 [SMILES: CCOC(=O)c1c(-c2ccco2)nc(N(C)C)nc1S(C)(=O)=O]

To a mixture of ethyl 2-amino-4-(2-furyl)-6-methylsulfonyl-pyrimidine-5-carboxylate (Int-10, 300.0 mg, 0.960 mmol) and iodomethane (410.35 mg, 2.89 mmol) in DMF (5 mL) was added sodium hydride (46.26 mg, 1.93 mmol) at 0° C. in ice bath. The mixture was stirred at 0° C. for 0.5 hr. After completion, the mixture was poured into water (30 mL), extracted with EtOAc (25 mL×2). The combined organic layer was washed with brine (25 mL×2), dried over Na₂SO₄ and purified by flash column ((eluting with EtOAc/PE=10%) to afford Compound 43a (100 mg) as a white solid. MS obsd. (ESI⁺) [(M+H)⁺]: 340.1.

### Step 2. Preparation of ethyl 4-(cyclopentylmethylamino)-2-(dimethylamino)-6-(2-furyl)pyrimidine-5-carboxylate

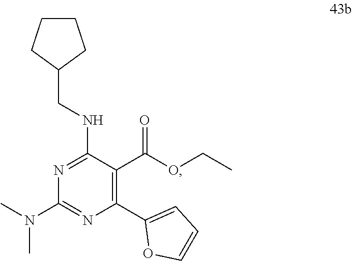 [SMILES: CCOC(=O)c1c(NCC2CCCC2)nc(N(C)C)nc1-c1ccco1]

The title compound was prepared in analogy to the procedure described for the preparation of Compound 37a, by using ethyl 2-(dimethylamino)-4-(2-furyl)-6-methylsulfonyl-pyrimidine-5-carboxylate (Compound 43a) and cyclopentylmethanamine instead of ethyl 2-amino-4-(furan-2-yl)-6-(methylsulfonyl)pyrimidine-5-carboxylate (Compound 37a) and (4-chlorophenyl)methanamine. The product was purified by preparative HPLC to afford Compound 43b (70 mg) as a white solid. MS obsd. (ESI⁺)[(M+H)⁺]: 359.2.

### Step 3: Preparation of 4-(cyclopentylmethylamino)-2-(dimethylamino)-6-(2-furyl)pyrimidine-5-carboxylic acid

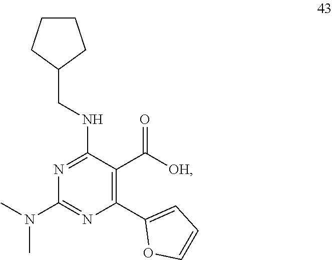 [SMILES: CN(C)c1nc(NCC2CCCC2)c(C(=O)O)c(-c2ccco2)n1]

The title compound was prepared in analogy to the procedure described for the preparation of Example 37, by using ethyl 4-(cyclopentylmethylamino)-2-(dimethylamino)-6-(2-furyl)pyrimidine-5-carboxylate (Compound 43b) instead of ethyl 2-amino-4-((4-chlorobenzyl)amino)-6-(furan-2-yl)pyrimidine-5-carboxylate (Compound 37a). The product was purified by preparative HPLC to afford Example 43 (5.1 mg) as a white solid. MS obsd. (ESI⁺)[(M+H)⁺]: 331.2. ¹H NMR (400 MHz, CDCl₃) δ ppm: 7.96 (s, 0.39H), 7.49 (s, 1H), 7.00 (dd, J=61.3, 2.8 Hz, 1H), 6.49 (s, 1H), 6.09 (s, 0.57H), 3.38-3.46 (m, 1H), 3.20, (d, J=14.7 Hz, 7H), 2.18 (dd, J=12.6, 7.4 Hz, 1H), 1.73-1.88 (m, 2H), 1.52-1.72 (m, 4H), 1.27 (m, 2H).

### Example 44

### Ethyl 2-amino-4-[(6-bromo-2,3-dimethoxy-phenyl)methylamino]-6-(2-furyl)pyrimidine-5-carboxylate

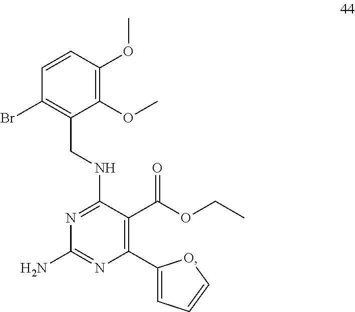 [SMILES: CCOC(=O)c1c(NCc2c(Br)ccc(OC)c2OC)nc(N)nc1-c1ccco1]

The title compound was prepared in analogy to the procedure described for the preparation of Compound 37a, by using (6-bromo-2,3-dimethoxy-phenyl)methanamine instead of (4-chlorophenyl)methanamine. The product was purified by preparative HPLC to afford Example 44 100 mg, 62%) as an off-white solid. MS obsd. (ESI⁺) [(M+H)⁺]: 478. ¹H NMR (400 MHz, DMSO-d₆)⁶ ppm: 7.79 (s, 1H), 7.67-7.54 (m, 1H), 7.39 (d, J=8.9 Hz, 1H), 7.06 (s, 3H), 6.86-6.79 (m, 1H), 6.64-6.56 (m, 1H), 4.71 (d, J=4.9 Hz, 2H), 3.99 (d, J=7.1 Hz, 2H), 3.83 (s, 3H), 3.77 (s, 3H), 0.95 (t, J=7.2 Hz, 3H).

### Example 45

### Ethyl 4-(2-furyl)-2-methylsulfanyl-6-[[3-(trifluoromethyl)phenyl]methylamino]pyrimidine-5-carboxylate

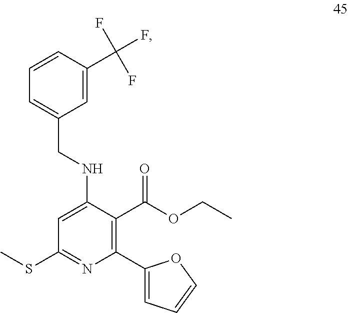 [SMILES: CCOC(=O)c1c(NCc2cccc(C(F)(F)F)c2)cc(SC)nc1-c1ccco1]

The title compound was prepared in analogy to the procedure described for the preparation of Compound 37a, by using ethyl 4-chloro-6-(2-furyl)-2-methylsulfanyl-pyrimidine-5-carboxylate (Int-8) and (3-(trifluoromethyl)phenyl)methanamine instead of ethyl 2-amino-4-(furan-2-yl)-6-(methylsulfonyl)pyrimidine-5-carboxylate (Int-10) and (4-chlorophenyl)methanamine. The product was purified by preparative HPLC to afford Example (2.8 g) as an off-white solid. MS obsd. (ESI⁺) [(M+H)⁺]: 438. ¹H NMR (400 MHz, DMSO-d₆) δ ppm: 8.08 (t, J=6.1 Hz, 1H), 7.88 (dd, J=0.7, 1.7 Hz, 1H), 7.70 (s, 1H), 7.65-7.54 (m, 3H), 7.11 (dd, J=0.7, 3.5 Hz, 1H), 6.67 (dd, J=1.7, 3.4 Hz, 1H), 4.69 (d, J=6.0 Hz, 2H), 4.29 (q, J=7.2 Hz, 2H), 2.37 (s, 3H), 1.18 (t, J=7.2 Hz, 3H).

### Example 46

### 4-(2-furyl)-2-(2-methoxyethylamino)-6-[[3-(trifluoromethyl)phenyl]methylamino]pyrimidine-5-carboxylic Acid

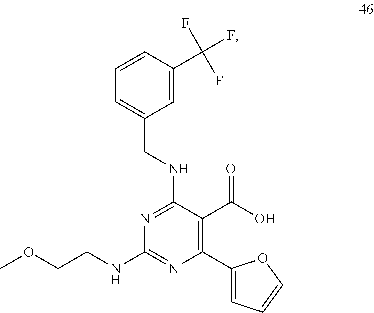 [SMILES: COCCNc1nc(NCc2cccc(C(F)(F)F)c2)c(C(=O)O)c(-c2ccco2)n1]

### Step 1: Preparation of ethyl 4-(2-furyl)-2-methylsulfonyl-6-[[3-(trifluoromethyl)phenyl]methylamino]pyrimidine-5-carboxylate

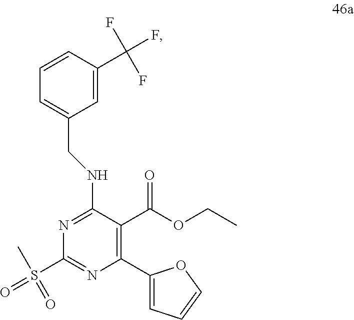 [SMILES: CCOC(=O)c1c(NCc2cccc(C(F)(F)F)c2)nc(S(C)(=O)=O)nc1-c1ccco1]

A mixture of ethyl 4-(2-furyl)-2-methylsulfanyl-6-[[3-(trifluoromethyl)phenyl]methylamino]pyrimidine-5-carboxylate (Example 45, 428 mg) and m-CPBA (439 mg, 1.96 mmol) in DCM (5 mL) was stirred at rt for 1 hr. The reaction mixture was diluted with DCM and washed with sat NaHCO₃ (3×10 mL). The organic layers were dried over MgSO4 and concentrated in vacuo to afford Compound 46a (410 mg). MS obsd. (ESI⁺) [(M+H)⁺]: 470.

### Step 2: Preparation of ethyl 4-(2-furyl)-2-(2-methoxyethylamino)-6-[[3-(trifluoromethyl)phenyl]methylamino]pyrimidine-5-carboxylate

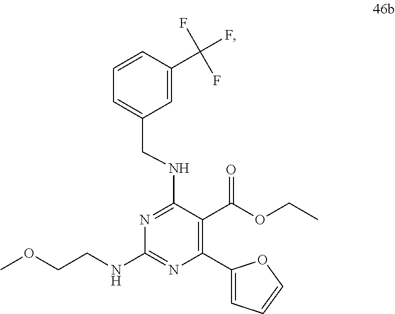 [SMILES: CCOC(=O)c1c(NCc2cccc(C(F)(F)F)c2)nc(NCCOC)nc1-c1ccco1]

A mixture of ethyl 4-(2-furyl)-2-methylsulfonyl-6-[[3-(trifluoromethyl)phenyl]methylamino]pyrimidine-5-carboxylate (Compound 46a, 300 mg), 2-methoxyethan-1-amine (48 mg) and N-ethyl-N-isopropylpropan-2-amine (82.6 mg) in THF (5 mL) was stirred at rt for 3 hrs. The crude reaction mixture was concentrated in vacuo to afford Compound 46b (300 mg) which was used in the next step without purification. MS obsd. (ESI⁺) [(M+H)⁺]: 465.

### Step 3: Preparation of 4-(2-furyl)-2-(2-methoxyethylamino)-6-[[3-(trifluoromethyl)phenyl]methylamino]pyrimidine-5-carboxylic Acid

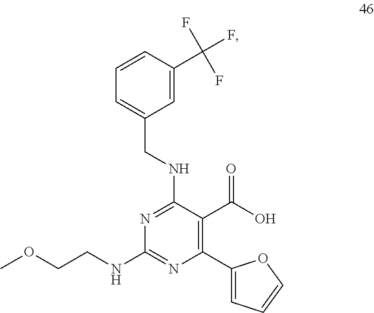 [SMILES: COCCNc1nc(NCc2cccc(C(F)(F)F)c2)c(C(=O)O)c(-c2ccco2)n1]

A mixture of ethyl 4-(furan-2-yl)-2-((2-methoxyethyl)amino)-6-((3-(trifluoromethyl)benzyl)amino)pyrimidine-5-carboxylate (Compound 46b, 300 mg) and KOH (181 mg, 3.23 mmol) in MeOH (0.5 mL)/H₂O (0.5 mL) was stirred at 90° C. for 1 hr. The reaction mixture was acidified to pH=7 with 1 M HCl. The crude material was purified by preparative HPLC to afford Example 46 (4.5 mg). MS obsd. (ESI⁺) [(M+H)⁺]: 437. ¹H NMR (400 MHz, DMSO-d₆) δ ppm: 8.59 (br s, 1H), 7.84 (br s, 1H), 7.52-7.72 (m, 4H), 7.38 (br s, 1H), 6.81-6.92 (m, 1H), 6.62 (br s, 1H), 4.71 (br d, J=5.7 Hz, 2H), 3.19-3.27 (m, 4H), 3.14 (br s, 3H).

### Example 47

### Ethyl 2-(2-azaspiro[3.3]heptan-2-yl)-4-(2-furyl)-6-[[3-(trifluoromethyl)phenyl]methylamino]pyrimidine-5-carboxylate

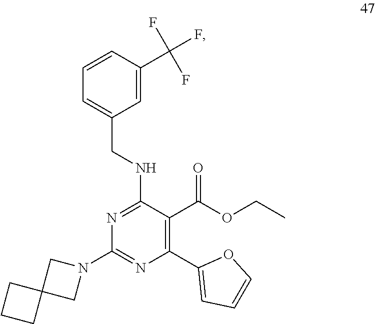 [SMILES: CCOC(=O)c1c(NCc2cccc(C(F)(F)F)c2)nc(N2CC3(CCC3)C2)nc1-c1ccco1]

The title compound was prepared in analogy to the procedure described for the preparation of Compound 46b, by using 2-azaspiro[3.3]heptane hemioxalate instead of 2-methoxyethan-1-amine. The product was purified by preparative HPLC to afford Example 47 (150 mg) as an off-white solid. MS obsd. (ESI⁺) [(M+H)⁺]: 487. ¹H NMR (400 MHz, DMSO-d₆) δ ppm: 8.01 (s, 1H), 7.79-7.73 (m, 2H), 7.67 (d, J=7.1 Hz, 1H), 7.63-7.54 (m, 2H), 6.84 (dd, J=0.7, 3.4 Hz, 1H), 6.59 (dd, J=1.8, 3.4 Hz, 1H), 5.76 (s, 3H), 4.61 (d, J=6.0 Hz, 1H), 4.67-4.56 (m, 1H), 4.10 (q, J=7.1 Hz, 2H), 3.94 (s, 4H), 2.17-2.06 (m, 4H), 1.79 (br t, J=7.5 Hz, 2H), 1.04 (t, J=7.2 Hz, 3H).

### Example 48

### 2-(2-azaspiro[3.3]heptan-2-yl)-4-(2-furyl)-6-[[3-(trifluoromethyl)phenyl]methylamino]pyrimidine-5-carboxylic Acid

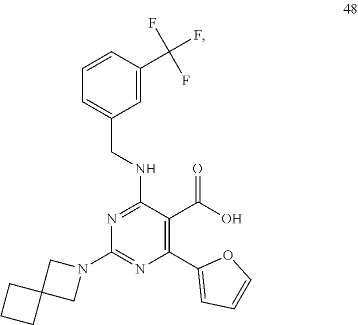 [SMILES: O=C(O)c1c(NCc2cccc(C(F)(F)F)c2)nc(N2CC3(CCC3)C2)nc1-c1ccco1]

The title compound was prepared in analogy to the procedure described for the preparation of Example 46, by using ethyl 4-(furan-2-yl)-2-(2-azaspiro[3.3]heptan-2-yl)-6-((3-(trifluoromethyl)benzyl)amino)pyrimidine-5-carboxylate (Example 47, 100 mg) instead of ethyl 4-(2-furyl)-2-(2-methoxyethylamino)-6-[[3-(trifluoromethyl)phenyl]methylamino]pyrimidine-5-carboxylate (Compound 46b). The product was purified by preparative HPLC to afford Example 48 (6 mg) as a white solid. MS obsd. (ESI⁺) [(M+H)⁺]: 459. ¹H NMR (400 MHz, DMSO-d₆) δ ppm: 8.42-8.24 (m, 1H), 7.81-7.70 (m, 2H), 7.69-7.50 (m, 3H), 6.85-6.73 (m, 1H), 6.60-6.46 (m, 1H), 4.68-4.55 (m, 2H), 3.92 (s, 4H), 2.12 (s, 4H), 1.78 (s, 2H).

### Example 49

### Ethyl 4-(2-furyl)-2-(isobutylamino)-6-[[3-(trifluoromethyl)phenyl]methylamino]pyrimidine-5-carboxylate

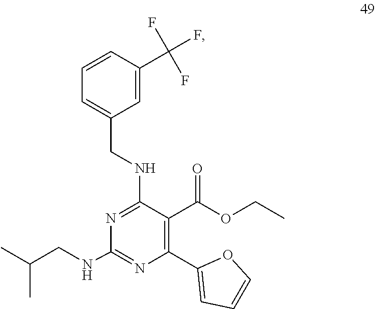 [SMILES: CCOC(=O)c1c(NCc2cccc(C(F)(F)F)c2)nc(NCC(C)C)nc1-c1ccco1]

The title compound was prepared in analogy to the procedure described for the preparation of Compound 46a, by using 2-methylpropan-1-amine instead of 2-methoxyethan-1-amine. The product was purified by preparative HPLC to afford Example 49 (150 mg) as a light yellow solid. MS obsd. (ESI⁺) [(M+H)⁺]: 463. ¹H NMR (400 MHz, DMSO-d₆) δ ppm: 8.22-8.08 (m, 1H), 7.84-7.84 (m, 1H), 7.77 (s, 1H), 7.69-7.63 (m, 1H), 7.62-7.53 (m, 3H), 7.45 (br s, 1H), 6.79 (br d, J=2.9 Hz, 1H), 6.59 (br s, 1H), 4.68 (br d, J=5.7 Hz, 2H), 4.08 (q, J=7.0 Hz, 2H), 2.93 (br t, J=6.2 Hz, 2H), 1.72-1.52 (m, 1H), 1.03 (t, J=7.1 Hz, 3H), 0.71 (br d, J=6.6 Hz, 6H).

### Example 50

### 4-(2-furyl)-2-(isobutylamino)-6-[[3-(trifluoromethyl)phenyl]methylamino]pyrimidine-5-carboxylic Acid

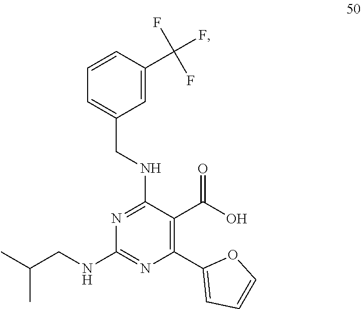 [SMILES: CC(C)CNc1nc(NCc2cccc(C(F)(F)F)c2)c(C(=O)O)c(-c2ccco2)n1]

The title compound was prepared in analogy to the procedure described for the preparation of Example 46, by using ethyl 4-(2-furyl)-2-(isobutylamino)-6-[[3-(trifluoromethyl)phenyl]methylamino]pyrimidine-5-carboxylate (Example 49) instead of ethyl 4-(2-furyl)-2-(2-methoxyethylamino)-6-[[3-(trifluoromethyl)phenyl]methylamino]pyrimidine-5-carboxylate (Compound 46b). The product was purified by preparative HPLC to afford Example 50 (7.5 mg) as a white solid. MS obsd. (ESI⁺) [(M+H)⁺]: 435. ¹H NMR (400 MHz, DMSO-d₆) δ ppm: 12.55-12.31 (m, 1H), 8.46-8.26 (m, 1H), 7.84-7.72 (m, 1H), 7.72-7.48 (m, 4H), 7.47-7.33 (m, 1H), 6.81-6.68 (m, 1H), 6.64-6.47 (m, 1H), 4.79-4.56 (m, 2H), 3.01-2.88 (m, 2H), 1.71-1.53 (m, 1H), 0.71 (br d, J=6.4 Hz, 6H).

### Example 51

### Step 1: Preparation of ethyl 4-(2-furyl)-2-(3-methoxypropylamino)-6-[[3-(trifluoromethyl)phenyl]methylamino]pyrimidine-5-carboxylate

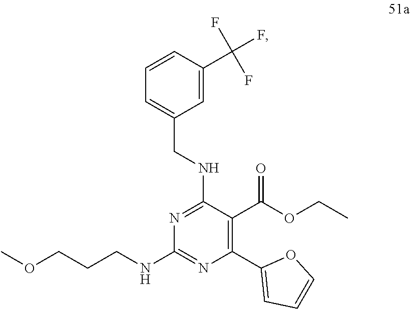 [SMILES: CCOC(=O)c1c(NCc2cccc(C(F)(F)F)c2)nc(NCCCOC)nc1-c1ccco1]

The title compound was prepared in analogy to the procedure described for the preparation of Compound 46b, by using 3-methoxypropan-1-amine instead of 2-methoxyethan-1-amine. The product was purified by preparative HPLC to afford Compound 51a (210 mg) as a yellow solid. Which was used in the next step directly without further purification. MS obsd. (ESI⁺) [(M+H)⁺]: 479.

### Step 2: Preparation of 4-(2-furyl)-2-(3-methoxypropylamino)-6-[[3-(trifluoromethyl)phenyl]methylamino]pyrimidine-5-carboxylic Acid

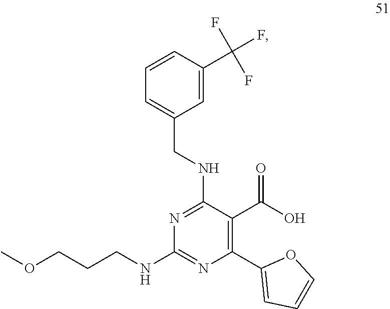 [SMILES: COCCCNc1nc(NCc2cccc(C(F)(F)F)c2)c(C(=O)O)c(-c2ccco2)n1]

The title compound was prepared in analogy to the procedure described for the preparation of Example 46, by using ethyl 4-(furan-2-yl)-2-((3-methoxypropyl)amino)-6-((3-(trifluoromethyl)benzyl)amino)pyrimidine-5-carboxylate (Compound 51a) instead of ethyl 4-(2-furyl)-2-(2-methoxyethylamino)-6-[[3-(trifluoromethyl)phenyl]methylamino]pyrimidine-5-carboxylate (Compound 46b). The product was purified by preparative HPLC to afford Example 51 (15 mg) as a white powder. MS obsd. (ESI⁺) [(M+H)⁺]: 451. ¹H NMR (400 MHz, DMSO-d₆) δ ppm: 8.68-8.37 (m, 1H), 7.78-7.47 (m, 5H), 7.23-6.87 (m, 1H), 6.87-6.69 (m, 1H), 6.61-6.37 (m, 1H), 4.75-4.61 (m, 2H), 3.18 (s, 7H), 1.76-1.48 (m, 2H).

### Example 52

### [2-amino-4-(2-furyl)-6-[[3-(trifluoromethyl)phenyl]methylamino]pyrimidin-5-yl]-(azetidin-1-yl)methanone

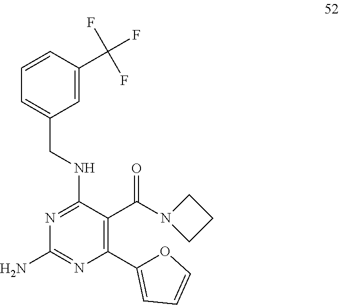 [SMILES: Nc1nc(NCc2cccc(C(F)(F)F)c2)c(C(=O)N2CCC2)c(-c2ccco2)n1]

To a solution of DIPEA (109 mg, 148 μl), 2-amino-4-(furan-2-yl)-6-((3-(trifluoromethyl)benzyl)amino)pyrimidine-5-carboxylic acid (Example 38, 80 mg) and 2-(3H-[1,2,3]triazolo[4,5-b]pyridin-3-yl)-1,1,3,3-tetramethylisouronium hexafluorophosphate (V) (161 mg) was added azetidine (12.1 mg) in DMF (5 ml) at rt. The resulting orange solution was stirred at rt for 3 hrs. The reaction mixture was concentrated and diluted with EtOAc, washed sequentially with water and sat. brine. The organic layer was dried over MgSO₄, filtered and evaporated. The crude material was purified by preparative HPLC to afford Example 52 (57 mg) as an off-white solid. MS obsd. (ESI⁺) [(M+H)⁺]: 418. ¹H NMR (400 MHz, DMSO-d₆) δ ppm: 7.85 (dd, J=0.9, 1.7 Hz, 1H), 7.70-7.53 (m, 4H), 7.20 (t, J=6.2 Hz, 1H), 6.91 (dd, J=0.8, 3.5 Hz, 1H), 6.63 (dd, J=1.7, 3.4 Hz, 1H), 6.34 (s, 2H), 4.63 (br s, 2H), 4.01 (br t, J=7.5 Hz, 2H), 3.80-3.40 (m, 2H), 2.08 (br s, 2H).

### Example 53

### [2-amino-4-(2-furyl)-6-[[3-(trifluoromethyl)phenyl]methylamino]pyrimidin-5-yl]-pyrrolidin-1-yl-methanone

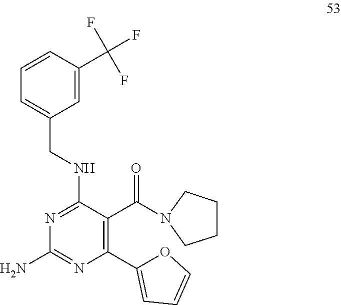 [SMILES: Nc1nc(NCc2cccc(C(F)(F)F)c2)c(C(=O)N2CCCC2)c(-c2ccco2)n1]

The title compound was prepared in analogy to the procedure described for the preparation of Example 52, by using pyrrolidine instead of azetidine. The product was purified by preparative HPLC to afford Example 53 (39 mg) as a white solid. MS obsd. (ESI⁺) [(M+H)⁺]: 432. ¹H NMR (400 MHz, DMSO-d₆) δ ppm: 7.76 (dd, J=0.8, 1.7 Hz, 1H), 7.66-7.60 (m, 2H), 7.59-7.52 (m, 2H), 7.04 (s, 1H), 6.85 (dd, J=0.7, 3.4 Hz, 1H), 6.58 (dd, J=1.8, 3.4 Hz, 1H), 6.25 (s, 2H), 4.63 (d, J=6.1 Hz, 2H), 3.53 (s, 1H), 3.46 (s, 1H), 3.12-3.00 (m, 1H), 2.86 (s, 1H), 1.95-1.63 (m, 4H).

### Example 54

### [2-amino-4-(2-furyl)-6-[[3-(trifluoromethyl)phenyl]methylamino]pyrimidin-5-yl]-(1-piperidyl)methanone

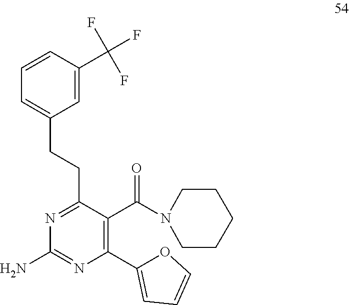 [SMILES: Nc1nc(CCc2cccc(C(F)(F)F)c2)c(C(=O)N2CCCCC2)c(-c2ccco2)n1]

The title compound was prepared in analogy to the procedure described for the preparation of Example 52, by using piperidine instead of azetidine. The product was purified by preparative HPLC to afford Example 54 (49 mg) as a white solid. MS obsd. (ESI⁺) [(M+H)⁺]: 446. ¹H NMR (400 MHz, DMSO-d₆) δ ppm: 7.79 (dd, J=0.8, 1.7 Hz, 1H), 7.64-7.52 (m, 4H), 6.95 (t, J=6.2 Hz, 1H), 6.87 (dd, J=0.8, 3.4 Hz, 1H), 6.59 (dd, J=1.7, 3.4 Hz, 1H), 6.23 (s, 2H), 4.62 (dd, J=6.1, 15.6 Hz, 2H), 3.82-3.41 (m, 2H), 3.19-2.96 (m, 2H), 1.50 (br s, 4H), 1.33-0.92 (m, 2H).

### Example 55

### [2-amino-4-(2-furyl)-6-[[3-(trifluoromethyl)phenyl]methylamino]pyrimidin-5-yl]-morpholino-methanone

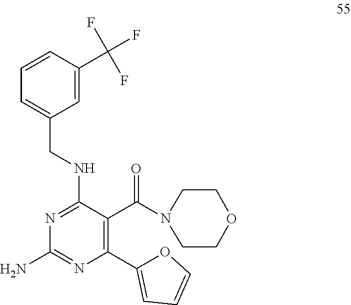 [SMILES: Nc1nc(NCc2cccc(C(F)(F)F)c2)c(C(=O)N2CCOCC2)c(-c2ccco2)n1]

The title compound was prepared in analogy to the procedure described for the preparation of Example 52, by using morpholine instead of azetidine. The product was purified by preparative HPLC to afford Example 55 (43 mg) as a white solid. MS obsd. (ESI⁺) [(M+H)⁺]: 448. ¹H NMR (400 MHz, DMSO-d₆) δ ppm: 7.83 (dd, J=0.7, 1.7 Hz, 1H), 7.65-7.52 (m, 4H), 7.04 (t, J=6.2 Hz, 1H), 6.89 (dd, J=0.8, 3.4 Hz, 1H), 6.61 (dd, J=1.7, 3.4 Hz, 1H), 6.28 (s, 2H), 4.64 (br d, J=5.4 Hz, 2H), 3.73-3.56 (m, 4H), 3.43-3.34 (m, 1H), 3.23-3.14 (m, 2H), 3.01 (br dd, J=6.4, 10.2 Hz, 1H).

### Example 56

### tert-butyl4-[2-amino-4-(2-furyl)-6-[[3-(trifluoromethyl)phenyl]methylamino]pyrimidine-5-carbonyl]piperazine-1-carboxylate

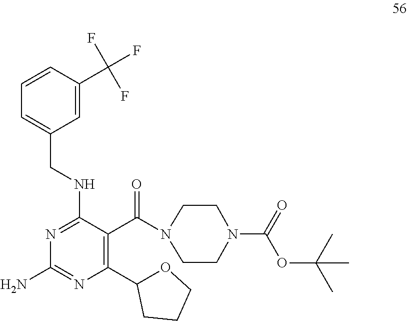 [SMILES: CC(C)(C)OC(=O)N1CCN(C(=O)c2c(NCc3cccc(C(F)(F)F)c3)nc(N)nc2C2CCCO2)CC1]

The title compound was prepared in analogy to the procedure described for the preparation of Example 52, by using tert-butyl piperazine-1-carboxylate instead of azetidine. The product was purified by preparative HPLC to afford Example 56 (40 mg) as a white solid. MS obsd. (ESI⁺) [(M+H)⁺]: 547. ¹H NMR (400 MHz, DMSO-d₆) δ ppm: 7.81 (dd, J=0.7, 1.7 Hz, 1H), 7.64-7.52 (m, 4H), 7.01 (s, 1H), 6.89 (dd, J=0.7, 3.4 Hz, 1H), 6.59 (dd, J=1.8, 3.4 Hz, 1H), 6.29 (s, 2H), 4.75-4.53 (m, 2H), 3.73-3.46 (m, 3H), 3.39-3.32 (m, 1H), 3.22-3.10 (m, 2H), 3.10-2.84 (m, 2H), 1.38 (s, 9H).

### Example 57

### Ethyl 4-[(2-chlorophenyl)methylamino]-6-(2-furyl)pyrimidine-5-carboxylate

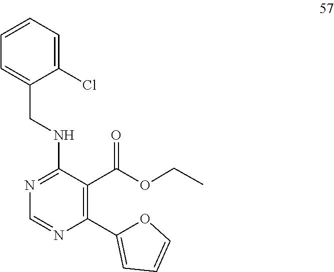 [SMILES: CCOC(=O)c1c(NCc2ccccc2Cl)ncnc1-c1ccco1]

The title compound was prepared in analogy to the procedure described for the preparation of Example 1, by using ethyl 4-chloro-6-(furan-2-yl)pyrimidine-5-carboxylate and (2-chlorophenyl)methanamine instead of 2-amino-4-chloro-6-(furan-2-yl)pyrimidine-5-carbonitrile (Int-1) and 1-phenylethanamine. The product was purified by preparative HPLC to afford Example 57 (10 mg) as a white solid. MS obsd. (ESI⁺) [(M+H)⁺]: 358. ¹H NMR (400 MHz, DMSO-d₆) δ ppm: 8.45 (s, 1H), 7.91 (dd, J=0.8, 1.8 Hz, 1H), 7.82 (t, J=5.9 Hz, 1H), 7.48-7.43 (m, 1H), 7.29 (s, 2H), 7.25-7.20 (m, 1H), 7.17 (dd, J=0.7, 3.4 Hz, 1H), 6.69 (dd, J=1.8, 3.5 Hz, 1H), 4.70 (d, J=6.0 Hz, 2H), 4.35 (q, J=7.1 Hz, 2H), 1.24 (t, J=7.1 Hz, 3H).

### Example 58

### 4-(2-furyl)-2-(2-methoxyethylamino)-6-[[1-[3-(trifluoromethyl)phenyl]cyclopropyl]amino]pyrimidine-5-carboxylic Acid

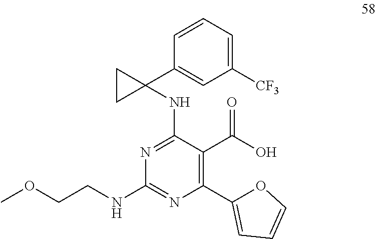 [SMILES: COCCNc1nc(NC2(c3cccc(C(F)(F)F)c3)CC2)c(C(=O)O)c(-c2ccco2)n1]

The title compound was prepared according to the following scheme:

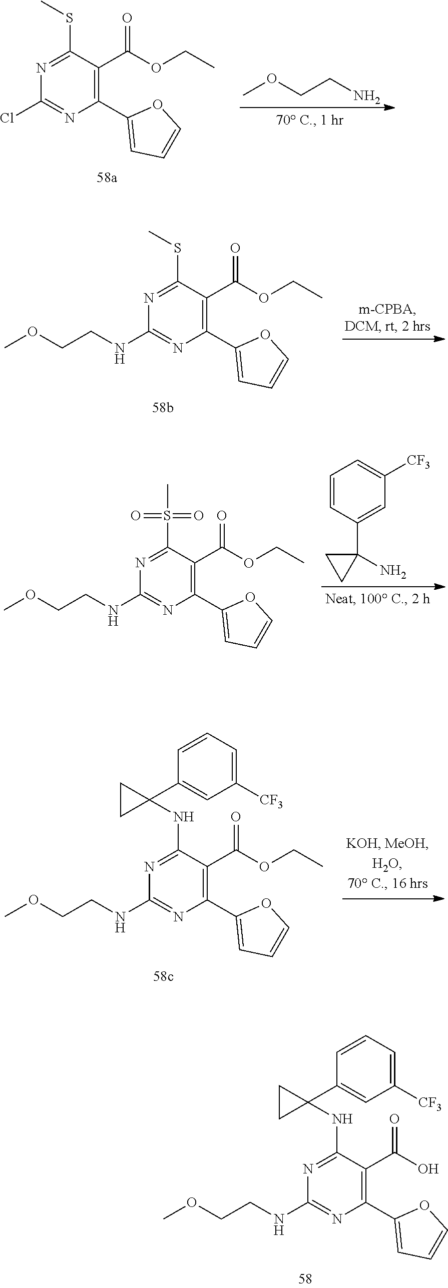

### Step 1: Preparation of ethyl 4-(2-furyl)-2-(2-methoxyethylamino)-6-methylsulfanyl-pyrimidine-5-carboxylate

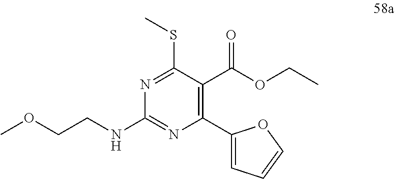 [SMILES: CCOC(=O)c1c(SC)nc(NCCOC)nc1-c1ccco1]

To a solution of ethyl 2-chloro-4-(2-furyl)-6-methylsulfanyl-pyrimidine-5-carboxylate (99.0 mg, 0.330 mmol) in MeCN (10 mL) was added triethylamine (0.23 mL, 1.66 mmol) and 2-methoxyethylamine (99.56 mg, 1.33 mmol). After being heated at 80° C. for 1 hr, the reaction mixture was concentrated by reduced pressure and purified by flash column ((eluting with EtOAc/PE=10%) to afford Compound 58a (91 mg, 81.39%) as a light yellow liquid. MS obsd. (ESI⁺)[(M+H)⁺]: 338.1.

### Step 2: Preparation of ethyl 4-(2-furyl)-2-(2-methoxyethylamino)-6-methylsulfonyl-pyrimidine-5-carboxylate

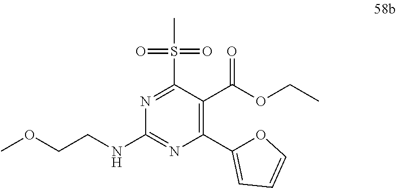 [SMILES: CCOC(=O)c1c(-c2ccco2)nc(NCCOC)nc1S(C)(=O)=O]

To a mixture of 3-chloroperoxybenzoic acid (124.29 mg, 0.720 mmol) in DCM (3 mL) was added ethyl 4-(2-furyl)-2-(2-methoxyethylamino)-6-methylsulfanyl-pyrimidine-5-carboxylate (81.0 mg, 0.240 mmol) in portions. The mixture was stirred at 25° C. for 3 hrs. After completion, the mixture was washed with Na₂SO₃ (sat. aq. 25 mL) and dried over Na₂SO₄, purified by flash column ((eluting with EtOAc/PE=0 to 70%) to afford Compound 58b (95 mg, 86.77%) as a yellow liquid. MS obsd. (ESI⁺)[(M+H)⁺]: 370.1.

### Step 3: Preparation of ethyl 4-(2-furyl)-2-(2-methoxyethylamino)-6-[[1-[3-(trifluoromethyl)phenyl]cyclopropyl]amino]pyrimidine-5-carboxylate

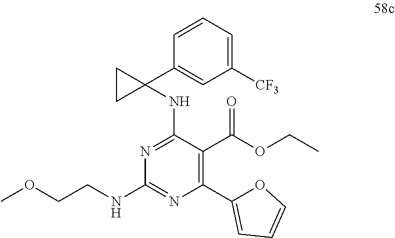 [SMILES: CCOC(=O)c1c(NC2(c3cccc(C(F)(F)F)c3)CC2)nc(NCCOC)nc1-c1ccco1]

The mixture of ethyl 4-(2-furyl)-2-(2-methoxyethylamino)-6-methylsulfonyl-pyrimidine-5-carboxylate (65.0 mg, 0.180 mmol), 1-[3-(trifluoromethyl)phenyl]cyclopropanamine (70.81 mg, 0.350 mmol) and DIPEA (45.48 mg, 0.350 mmol) were stirred at 100° C. for 4 hrs. The crude product was purified by flash column chromatography ((eluting with EtOAc/PE=0 to 60%) to afford Compound 58c (62 mg) as a colorless oil. MS obsd. (ESI⁺)[(M+H)⁺]: 491.2.

### Step 4: Preparation of 4-(2-furyl)-2-(2-methoxyethylamino)-6-[[1-[3-(trifluoromethyl)phenyl]cyclopropyl]amino]pyrimidine-5-carboxylic Acid

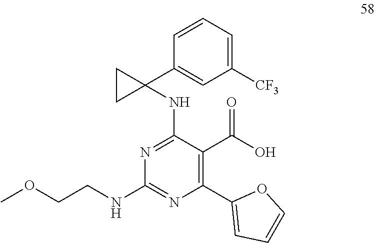 [SMILES: COCCNc1nc(NC2(c3cccc(C(F)(F)F)c3)CC2)c(C(=O)O)c(-c2ccco2)n1]

To the mixture of ethyl 4-(2-furyl)-2-(2-methoxyethylamino)-6-[[1-[3-(trifluoromethyl)phenyl]cyclopropyl]amino]pyrimidine-5-carboxylate (Compound 58c, 62.0 mg, 0.130 mmol) in methanol (15 mL) and water (3 mL) was added potassium hydroxide (21.28 mg, 0.380 mmol). After being heated to 70° C. and stirred at this temperature for 16 hrs, the mixture was concentrated. The aqueous phase was acidified to pH=7 with 1 M HCl and purified by prep-HPLC (Column: Gemini C18 100×21.2 mm, 5 μm; Mobile phase: MeCN˜ H₂O (0.1% FA); Rate: 25 mL/min; Gradient: 30-40) to afford Example 58 (23 mg) as a white solid. MS obsd. (ESI⁺): 463.1 [(M+H)⁺]. ¹H NMR (400 MHz, DMSO-d₆) δ ppm: 8.97 (s, 1H), 7.96 (s, 1H), 7.53 (d, J=6.3 Hz, 5H), 7.16 (s, 1H), 6.72 (s, 1H), 3.08 (d, J=15.6 Hz, 7H), 1.39 (d, J=23.3 Hz, 4H). ¹⁹F NMR (376 MHz, DMSO-d₆) δ ppm: −60.92.

### Example 59

### 4-[(3-chlorophenyl)methylamino]-6-(2-furyl)-2-(2-methoxyethylamino)pyrimidine-5-carboxylic Acid

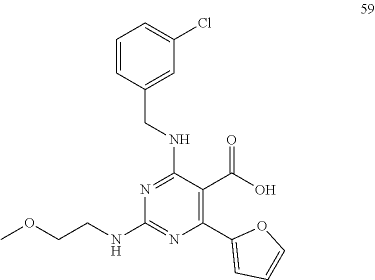 [SMILES: COCCNc1nc(NCc2cccc(Cl)c2)c(C(=O)O)c(-c2ccco2)n1]

### Step 1: Preparation of ethyl 4-[(3-chlorophenyl)methylamino]-6-(2-furyl)-2-(2-methoxyethylamino)pyrimidine-5-carboxylate

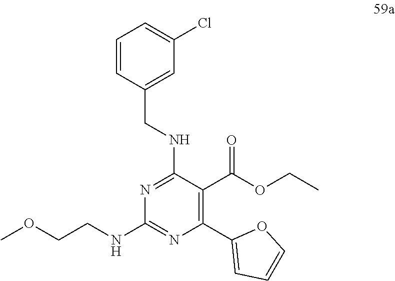 [SMILES: CCOC(=O)c1c(NCc2cccc(Cl)c2)nc(NCCOC)nc1-c1ccco1]

The title compound was prepared in analogy to the procedure described for the preparation of Compound 58c, by 3-chlorobenzylamine instead of 1-[3-(trifluoromethyl)phenyl]cyclopropanamine. The product was purified by preparative HPLC to afford Compound 59a (124 mg) as colorless oil. MS obsd. (ESI⁺) [(M+H)⁺]: 433.1

### Step 2: Preparation of 4-[(3-chlorophenyl)methylamino]-6-(2-furyl)-2-(2-methoxyethylamino)pyrimidine-5-carboxylic Acid

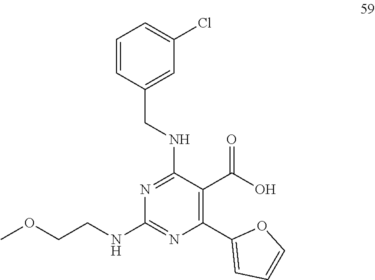 [SMILES: COCCNc1nc(NCc2cccc(Cl)c2)c(C(=O)O)c(-c2ccco2)n1]

The title compound was prepared in analogy to the procedure described for the preparation of Example 58, by using ethyl 4-[(3-chlorophenyl)methylamino]-6-(2-furyl)-2-(2-methoxyethylamino)pyrimidine-5-carboxylate (compound 59a) instead of ethyl 4-(2-furyl)-2-(2-methoxyethylamino)-6-[[1-[3-(trifluoromethyl)phenyl]cyclopropyl]amino]pyrimidine-5-carboxylate (compound 58c. The product was purified by preparative HPLC to afford Example (70.4 mg, 64.31%) as a light yellow solid. MS obsd. (ESI⁺)[(M+H)⁺]: 403.1. ¹H NMR (400 MHz, CD₃OD) δ ppm: 7.59 (dd, J=1.7, 0.7 Hz, 1H), 7.36 (s, 1H), 7.25-7.29 (m, 2H), 7.17-7.23 (m, 1H), 7.03 (d, J=3.0 Hz, 1H), 6.52 (dd, J=3.5, 1.8 Hz, 1H), 4.63 (s, 2H), 3.48 (t, J=5.4 Hz, 2H), 3.38 (t, J=5.4 Hz, 2H), 3.29 (s, 3H).

### Example 60

### 2-(ethylamino)-4-(2-furyl)-6-[[1-[3-(trifluoromethyl)phenyl]cyclopropyl]amino]pyrimidine-5-carboxylic Acid

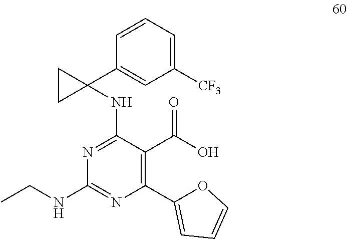 [SMILES: CCNc1nc(NC2(c3cccc(C(F)(F)F)c3)CC2)c(C(=O)O)c(-c2ccco2)n1]

The title compound was prepared according to the following scheme:

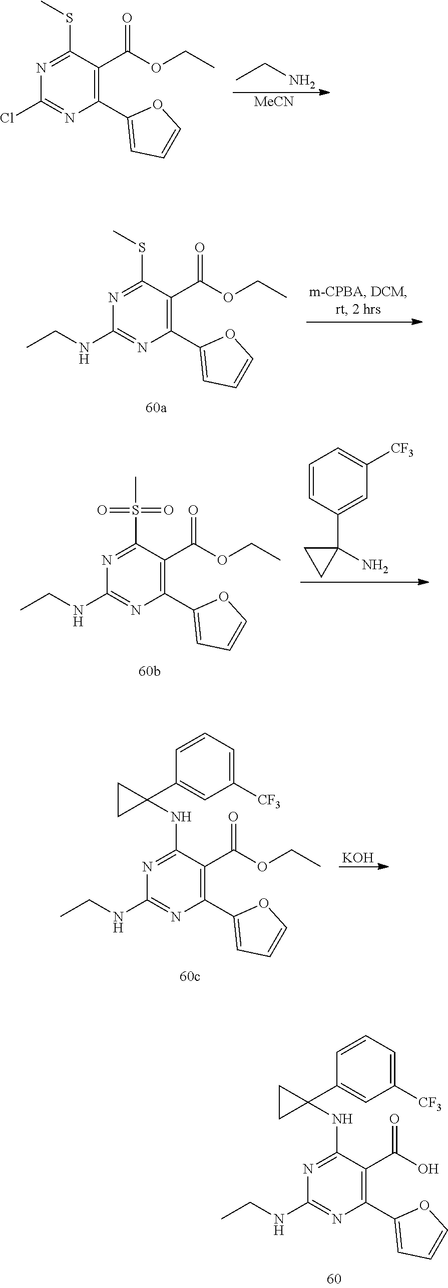

### Step 1: Preparation of ethyl 2-(ethylamino)-4-(2-furyl)-6-methylsulfanyl-pyrimidine-5-carboxylate

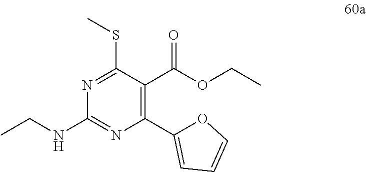 [SMILES: CCNc1nc(SC)c(C(=O)OCC)c(-c2ccco2)n1]

The title compound was prepared in analogy to the procedure described for the preparation of Compound 58a, by using ethylamine instead of 2-methoxyethylamine. The product was purified by preparative HPLC to afford Compound 60a (86 mg, 83.59%) as a light yellow liquid. MS obsd. (ESI⁺)[(M+H)⁺]: 308.1.

### Step 2: Preparation of ethyl 2-(ethylamino)-4-(2-furyl)-6-methylsulfonyl-pyrimidine-5-carboxylate

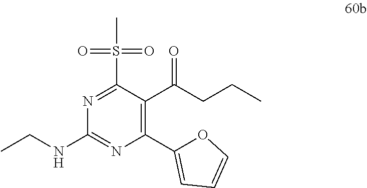 [SMILES: CCCC(=O)c1c(-c2ccco2)nc(NCC)nc1S(C)(=O)=O]

The title compound was prepared in analogy to the procedure described for the preparation of Compound 58b, by using ethyl 2-(ethylamino)-4-(2-furyl)-6-methylsulfanyl-pyrimidine-5-carboxylate instead of ethyl 4-(2-furyl)-2-(2-methoxyethylamino)-6-methylsulfanyl-pyrimidine-5-carboxylate. The product was purified by preparative HPLC to afford Compound 60b (45 mg) as a light yellow liquid. MS obsd. (ESI⁺)[(M+H)⁺]: 340.1.

### Step 3: Preparation of ethyl 2-(ethylamino)-4-(2-furyl)-6-[[1-[3-(trifluoromethyl)phenyl]cyclopropyl]amino]pyrimidine-5-carboxylate

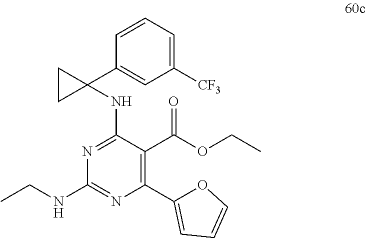 [SMILES: CCNc1nc(NC2(c3cccc(C(F)(F)F)c3)CC2)c(C(=O)OCC)c(-c2ccco2)n1]

The title compound was prepared in analogy to the procedure described for the preparation of compound 58c, by using ethyl 2-(ethylamino)-4-(2-furyl)-6-methylsulfonyl-pyrimidine-5-carboxylate (Compound 60b) instead of ethyl 4-(2-furyl)-2-(2-methoxyethylamino)-6-methylsulfonyl-pyrimidine-5-carboxylate (Compound 58b). The product was purified by preparative HPLC to afford Compound 60c (17 mg) as a colorless liquid. MS obsd. (ESI⁺)[(M+H)⁺]: 461.2.

### Step 4: Preparation of 2-(ethylamino)-4-(2-furyl)-6-[[1-[3-(trifluoromethyl)phenyl]cyclopropyl]amino]pyrimidine-5-carboxylic Acid

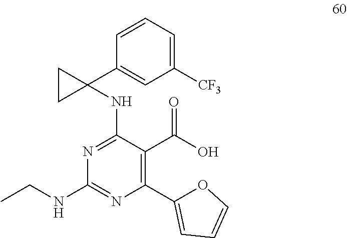 [SMILES: CCNc1nc(NC2(c3cccc(C(F)(F)F)c3)CC2)c(C(=O)O)c(-c2ccco2)n1]

The title compound was prepared in analogy to the procedure described for the preparation of Example 58, by using ethyl 2-(ethylamino)-4-(2-furyl)-6-[[1-[3-(trifluoromethyl)phenyl]cyclopropyl]amino]pyrimidine-5-carboxylate (Compound 60c) instead of ethyl 4-(2-furyl)-2-(2-methoxyethylamino)-6-[[1-[3-(trifluoromethyl)phenyl]cyclopropyl]amino]pyrimidine-5-carboxylate (Compound 58c). The product was purified by preparative HPLC to afford Example 60 (12 mg) as a white solid. MS obsd. (ESI⁺)[(M+H)⁺]: 433.2. ¹H NMR (400 MHz, DMSO-d₆) δ ppm: 8.53 (s, 1H), 7.74 (s, 1H), 7.42-7.62 (m, 4H), 7.25 (s, 1H), 0.80 (s, 3H), 6.76 (s, 1H), 6.55 (s, 1H), 3.06 (s, 2H), 1.34 (d, J=30.1 Hz, 4H). ¹⁹F NMR (376 MHz, DMSO-d₆) δ ppm: −60.94.

### Example 61

### Ethyl 4-(2-furyl)-2-methyl-6-[[3-(trifluoromethyl)phenyl]methylamino]pyrimidine-5-carboxylate

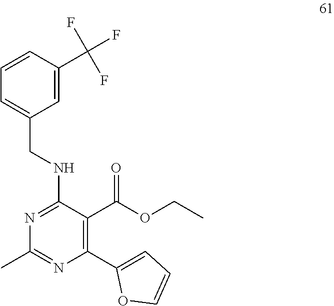 [SMILES: CCOC(=O)c1c(NCc2cccc(C(F)(F)F)c2)nc(C)nc1-c1ccco1]

The title compound was prepared in analogy to the procedure described for the preparation of Example 1, by using ethyl 4-chloro-6-(2-furyl)-2-methyl-pyrimidine-5-carboxylate (Int-11) and 3-(trifluoromethyl)benzylamine instead of 2-amino-4-chloro-6-(furan-2-yl)pyrimidine-5-carbonitrile (Int-1) and 1-phenylethanamine. The product was purified by preparative HPLC to afford Example 61 (120 mg) as a white solid. MS (ESI⁺) [(M+H)⁺]: 406.1. ¹H NMR (400 MHz, CDCl₃) δ ppm: 7.63 (s, 2H), 7.49-7.58 (m, 3H), 7.41-7.49 (m, 1H), 7.11 (s, 1H), 6.54 (dd, J=3.4, 1.7 Hz, 1H), 4.81 (d, J=5.8 Hz, 2H), 4.21 (q, J=7.1 Hz, 2H), 2.57 (s, 3H), 1.13 (t, J=7.1 Hz, 3H).

### Example 62

### Ethyl 4-[(6-bromo-2,3-dimethoxy-phenyl)methylamino]-6-(2-furyl)-2-isopropyl-pyrimidine-5-carboxylate

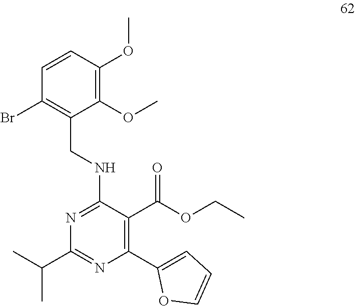 [SMILES: CCOC(=O)c1c(NCc2c(Br)ccc(OC)c2OC)nc(C(C)C)nc1-c1ccco1]

The title compound was prepared in analogy to the procedure described for the preparation of Example 1, by using ethyl 4-chloro-6-(2-furyl)-2-isopropyl-pyrimidine-5-carboxylate (Int-12) and (6-bromo-2,3-dimethoxy-phenyl)methanamine instead of 2-amino-4-chloro-6-(furan-2-yl)pyrimidine-5-carbonitrile (Int-1) and 1-phenylethanamine. The product was purified by preparative HPLC to afford Example 62 (130 mg) as a yellow oil. MS obsd. (ESI⁺)[(M+H)⁺]: 504.1. ¹H NMR (400 MHz, CDCl₃) δ ppm: 7.54 (s, 1H), 7.27-7.38 (m, 1H), 7.20 (s, 1H) 7.14 (s, 1H), 6.99 (s, 1H), 6.59 (s, 1H), 4.75 (d, J=6.0 Hz, 2H), 4.23 (dd, J=14.4, 7.3 Hz, 2H), 3.86-3.95 (m, 7H), 1.36 (d, J=6.3 Hz, 6H), 1.13 (t, J=7.0 Hz, 3H).

### BIOLOGICAL EXAMPLES

### BIO-Example 1: Cryopreserved Primary Human Hepatocytes (PHH) Assay

This assay is used to confirm the anti-HBV effect of the compounds in HBV PHH infection assay. Cryopreserved PHH (BioreclamationIVT, Lot YJM) was thawed at 37° C. and gently transferred into pre-warmed InVitroGRO HT medium (BioreclamationIVT, Cat. S03317). The mixture was centrifuged at 70 relative centrifugal force (RCF) for 3 min at RT, and the supernatant was discarded. Pre-warmed InVitroGRO CP medium (BioreclamationIVT, Cat #S03316) was added to the cell pellet to gently re-suspend cells. The cells were seeded at the density of 5.8×10⁴ cells per well to collagen I coated 96-well plate (Gibco, Cat. A1142803) with the InVitroGRO CP medium. All plates were incubated at 37° C. with 5% CO₂ and 85% humidity.

At 20 hrs after plating, the medium was changed to PHH culture medium (Dulbecco's Modified Eagle Medium (DMEM)/F12 (1:1) (Gibco, Cat. 11320-033), 10% fetal bovine serum (Gibco Cat. 10099141), 100 U/mL penicillin, 100 μg/mL streptomycin (Gibco, Cat. 151401-122), ng/mL human epidermal growth factor (Invitrogen Cat. PHG0311L), 20 ng/mL dexamethasone (Sigma, Cat. D4902) and 250 ng/mL human recombinant insulin (Gibco, Cat. 12585-014)). And the cells were incubated at 37° C. with 5% CO₂ and 85% humidity for 4 hrs. The medium was then changed to pre-warmed PHH culture medium containing 4% polyethylene glycol (PEG) MW8000 (Sigma, Cat. P1458-50 ML) and 1% DMSO (Sigma, Cat. D2650). 5.8×10⁶ genomic equivalents of HBV were added into the medium.

At 24 hrs post-infection, the cells were gently washed with PBS and refreshed with PHH culture medium supplemented with 1% DMSO, and 0.25 mg/mL Matrix gel (Corning, Cat. 356237) at 200 μL per well. All plates were immediately placed in at 37° C. CO₂ incubator.

24 hrs later, serial dilutions of compounds made with DMSO were further diluted with the same culture medium (PHH culture medium supplemented with 1% DMSO and 0.25 mg/mL Matrix gel as described above) before they were added to the cells to reach desired final compound concentrations and 1% DMSO concentration. The medium containing the compounds were refreshed every three days.

At 9 days post-compound treatment, extracellular HBsAg level were measured with Chemiluminescence Immuno Assay (CLIA) kit (Autobio, HBsAg Quantitative CLIA).

HBsAg IC₅₀ was derived from the dose-response curve using 4 parameter logistic curve fit method. The compounds of formula (I) have HBsAg IC₅₀<20 μM, particularly <1 μM. Results of Cryopreserved PHH assay are given in Table 1.

TABLE 1
HBsAg IC₅₀ data in Cryopreserved PHH assay

| Example | HBsAg | Example | HBsAg |
|---|---|---|---|
| No. | IC₅₀ (μM) | No. | IC50 (μM) |
| 1 | 5.93 | 32 | 3.49 |
| 2 | 3.28 | 33 | 7.58 |
| 3 | 1.67 | 34 | 3.49 |
| 4 | 11.4 | 35 | 7.85 |
| 5 | 0.47 | 36 | 6.62 |
| 6 | 10.1 | 37 | 2.93 |
| 7 | 1.78 | 38 | 2.49 |
| 8 | 10.24 | 39 | 9.07 |
| 9 | 8.68 | 40 | 6.67 |
| 10 | 3.62 | 41 | 3.24 |
| 11 | 2.35 | 42 | 5.27 |
| 12 | 3.89 | 43 | 5.55 |
| 13 | 3.93 | 44 | 8.06 |
| 14 | 2.76 | 45 | 8.16 |
| 15 | 3.01 | 46 | 4.31 |
| 16 | 6.75 | 47 | 3.26 |
| 17 | 3.35 | 48 | 6.37 |
| 18 | 6.02 | 49 | 5.35 |
| 19 | 2.42 | 50 | 8.62 |
| 20 | 2.66 | 51 | 9.61 |
| 21 | 6.66 | 52 | 7.94 |
| 22 | 2.73 | 53 | 9.25 |
| 23 | 6.89 | 54 | 6.59 |
| 24 | 3.07 | 55 | 10.1 |
| 25 | 2.03 | 56 | 9.66 |
| 26 | 5.7 | 57 | 4.26 |
| 27 | 6.83 | 58 | 5.96 |
| 28 | 0.42 | 59 | 3.71 |
| 29 | 2.61 | 60 | 6.5 |
| 30 | 9.06 | 61 | 7.38 |
| 31 | 0.27 | 62 | 10.2 |

1. A compound of the formula (I), 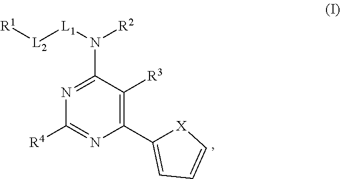
wherein
R¹ is C₁₋₆ alkyl, C₃₋₇ cycloalkyl, phenyl or 4-6 membered heterocyclyl; wherein C₁₋₆ alkyl, C₃₋₇ cycloalkyl, phenyl and 4-6 membered heterocyclyl are unsubstituted or substituted by one or two or three or four substituents independently selected from C₁₋₆ alkyl, C₁₋₆ alkoxy, halogen, haloC₁₋₆ alkyl, C₁₋₆ alkylsulfonyl and amino;
R² is H, halogen, C₁₋₆ alkyl or haloC₁₋₆ alkyl;
R³ is H, halogen, C₁₋₆ alkyl, haloC₁₋₆ alkyl, CN, C₁₋₆ alkoxy, carboxy, C₁₋₆ alkoxycarbonyl, (4-6 membered heterocyclyl)carbonyl or C₁₋₆ alkoxycarbonyl(4-6 membered heterocyclyl)carbonyl;
R⁴ is H, halogen, C₁₋₆ alkyl, haloC₁₋₆ alkyl, C₁₋₆ alkoxy, amino, C₁₋₆ alkylamino, (C₁₋₆ alkyl)₂ amino, haloC₁₋₆ alkylamino, C₁₋₆ alkoxyC₁₋₆ alkylamino, C₁₋₆ alkylsulfanyl or 2-azaspiro[3.3]heptanyl;
L₁ is a bond, —CH₂—, —CH₂CH₂—, —CH(CH₃)—, —CH₂CH₂CH₂— or —CH₂CH(halogen)CH₂—;
L₂ is a bond, 4-6 membered heterocyclyl or C₃₋₇ cycloalkyl;
X is NH, O or S;
or a pharmaceutically acceptable salt thereof.

2. A compound according to claim 1, or a pharmaceutically acceptable salt thereof, wherein R¹ is C₁₋₆ alkyl, C₃₋₇ cycloalkyl, phenyl or tetrahydropyranyl; wherein C₃₋₇ cycloalkyl, phenyl and tetrahydropyranyl are unsubstituted or substituted by one or two or three or four substituents independently selected from C₁₋₆ alkyl, C₁₋₆ alkoxy, halogen, haloC₁₋₆ alkyl, C₁₋₆ alkylsulfonyl and amino.

3. A compound according to claim 1 or 2, or a pharmaceutically acceptable salt thereof, wherein R¹ is C₃₋₇ cycloalkyl, phenyl or tetrahydropyranyl; wherein phenyl and tetrahydropyranyl are unsubstituted or substituted by one or two or three or four substituents independently selected from C₁₋₆ alkyl, halogen, haloC₁₋₆ alkyl, C₁₋₆ alkylsulfonyl and amino.

4. A compound according to any one of claims 1-3, or a pharmaceutically acceptable salt thereof, R¹ is cyclopentyl, phenyl or tetrahydropyranyl; wherein phenyl and tetrahydropyranyl are unsubstituted or substituted by one or two or three or four substituents independently selected from methyl, Cl, CF₃, methylsulfonyl and amino.

5. A compound according to any one of claims 1-4, or a pharmaceutically acceptable salt thereof, wherein R² is H or C₁₋₆ alkyl.

6. A compound according to any one of claims 1-5, or a pharmaceutically acceptable salt thereof, wherein R² is H.

7. A compound according to any one of claims 1-6, or a pharmaceutically acceptable salt thereof, wherein R³ is H, CN, C₁₋₆ alkoxy, carboxy, C₁₋₆ alkoxycarbonyl, azetidinylcarbonyl, pyrrolidinylcarbonyl, piperidylcarbonyl, morpholinocarbonyl or C₁₋₆ alkoxycarbonylpiperazinylcarbonyl.

8. A compound according to any one of claims 1-7, or a pharmaceutically acceptable salt thereof, wherein R³ is CN or carboxy.

9. A compound according to any one of claims 1-8, or a pharmaceutically acceptable salt thereof, wherein R⁴ is H, C₁₋₆ alkyl, C₁₋₆ alkoxy, amino, C₁₋₆ alkylamino, (C₁₋₆ alkyl)₂ amino, haloC₁₋₆ alkylamino, C₁₋₆ alkoxyC₁₋₆ alkylamino, C₁₋₆ alkylsulfanyl or 2-azaspiro[3.3]heptanyl.

10. A compound according to any one of claims 1-9, or a pharmaceutically acceptable salt thereof, wherein R⁴ is H, C₁₋₆ alkyl, C₁₋₆ alkoxy, amino, C₁₋₆ alkylamino or C₁₋₆ alkoxyC₁₋₆ alkylamino.

11. A compound according to any one of claims 1-10, or a pharmaceutically acceptable salt thereof, wherein R⁴ is H, methyl, butoxy, amino, ethylamino or methoxyethylamino.

12. A compound according to any one of claims 1-11, or a pharmaceutically acceptable salt thereof, wherein L₁ is a bond, —CH₂—, —CH₂CH₂—, —CH(CH₃)— or —CH₂CH(F)CH₂—.

13. A compound according to any one of claims 1-12, or a pharmaceutically acceptable salt thereof, wherein L₁ is a bond, —CH₂— or —CH(CH₃)—.

14. A compound according to any one of claims 1-13, or a pharmaceutically acceptable salt thereof, wherein L₂ is a bond or C₃₋₇ cycloalkyl.

15. A compound according to any one of claims 1-14, or a pharmaceutically acceptable salt thereof, wherein L₂ is a bond or cyclopropyl.

16. A compound according to any one of claims 1-15, or a pharmaceutically acceptable salt thereof, wherein X is O or S.

17. A compound according to any one of claims 1-16, or a pharmaceutically acceptable salt thereof, wherein X is O.

18. A compound according to claim 1, wherein
R¹ is C₁₋₆ alkyl, C₃₋₇ cycloalkyl, phenyl or tetrahydropyranyl; wherein C₃₋₇ cycloalkyl, phenyl and tetrahydropyranyl are unsubstituted or substituted by one or two or three or four substituents independently selected from C₁₋₆ alkyl, C₁₋₆ alkoxy, halogen, haloC₁₋₆ alkyl, C₁₋₆ alkylsulfonyl and amino;
R² is H or C₁₋₆ alkyl;
R³ is H, CN, C₁₋₆ alkoxy, carboxy, C₁₋₆ alkoxycarbonyl, azetidinylcarbonyl, pyrrolidinylcarbonyl, piperidylcarbonyl, morpholinocarbonyl or C₁₋₆ alkoxycarbonylpiperazinylcarbonyl;
R⁴ is H, C₁₋₆ alkyl, C₁₋₆ alkoxy, amino, C₁₋₆ alkylamino, (C₁₋₆ alkyl)₂ amino, haloC₁₋₆ alkylamino, C₁₋₆ alkoxyC₁₋₆ alkylamino, C₁₋₆ alkylsulfanyl or 2-azaspiro[3.3]heptanyl;
L₁ is a bond, —CH₂—, —CH₂CH₂—, —CH(CH₃)— or —CH₂CH(F)CH₂—;
L₂ is a bond or C₃₋₇ cycloalkyl;
X is O or S;
or a pharmaceutically acceptable salt thereof.

19. A compound according to claim 1, wherein
R¹ is C₃₋₇ cycloalkyl, phenyl or tetrahydropyranyl; wherein phenyl and tetrahydropyranyl are unsubstituted or substituted by one or two or three or four substituents independently selected from C₁₋₆ alkyl, halogen, haloC₁₋₆ alkyl, C₁₋₆ alkylsulfonyl and amino;
R² is H;
R³ is CN or carboxy;
R⁴ is H, C₁₋₆ alkyl, C₁₋₆ alkoxy, amino, C₁₋₆ alkylamino or C₁₋₆ alkoxyC₁₋₆ alkylamino;
L₁ is a bond, —CH₂— or —CH(CH₃)—;
L₂ is a bond or C₃₋₇ cycloalkyl;
X is O;
or a pharmaceutically acceptable salt thereof.

20. A compound according to claim 1, wherein
R¹ is cyclopentyl, phenyl or tetrahydropyranyl; wherein phenyl and tetrahydropyranyl are unsubstituted or substituted by one or two or three or four substituents independently selected from methyl, Cl, CF₃, methylsulfonyl and amino;
R² is H;
R³ is CN or carboxy;
R⁴ is H, methyl, butoxy, amino, ethylamino or methoxyethylamino;
L₁ is a bond, —CH₂— or —CH(CH₃)—;
L₂ is a bond or cyclopropyl;
X is O;
or a pharmaceutically acceptable salt thereof.

21. A compound according to claim 1, selected from
2-amino-4-(2-furyl)-6-(1-phenylethylamino)pyrimidine-5-carbonitrile;
2-amino-4-[(3-bromo-4-isopropoxy-phenyl)methylamino]-6-(2-furyl)pyrimidine-5-carbonitrile;
2-amino-4-(2-furyl)-6-[(2,2,6,6-tetramethyltetrahydropyran-4-yl)methylamino]pyrimidine-5-carbonitrile;
2-amino-4-[[3-(2-chlorophenyl)-2-fluoro-propyl]amino]-6-(2-furyl)pyrimidine-5-carbonitrile;
2-amino-4-(2-furyl)-6-[1-[3-(trifluoromethyl)phenyl]ethylamino]pyrimidine-5-carbonitrile;
2-amino-4-(2-furyl)-6-[(3-methoxyphenyl)methyl-methyl-amino]pyrimidine-5-carbonitrile;
2-amino-4-(2-furyl)-6-[[1-(2-methylsulfonylphenyl)cyclopropyl]amino]pyrimidine-5-carbonitrile;
2-amino-4-[(3,3-difluorocyclopentyl)methylamino]-6-(2-furyl)pyrimidine-5-carbonitrile;
2-amino-4-(cyclopentylamino)-6-(2-furyl)pyrimidine-5-carbonitrile;
2-amino-4-(cyclohexylmethylamino)-6-(2-furyl)pyrimidine-5-carbonitrile;
4-(2-furyl)-2-methylsulfanyl-6-[[3-(trifluoromethyl)phenyl]methylamino]pyrimidine-5-carbonitrile;
4-(2-furyl)-2-(2-methoxyethylamino)-6-[[3-(trifluoromethyl)phenyl]methylamino]pyrimidine-5-carbonitrile;
4-(2-furyl)-2-(2,2,2-trifluoroethylamino)-6-[[3-(trifluoromethyl)phenyl]methylamino]pyrimidine-5-carbonitrile;
2-(ethylamino)-4-(2-furyl)-6-[[3-(trifluoromethyl)phenyl]methylamino]pyrimidine-5-carbonitrile;
4-(2-furyl)-2-(propylamino)-6-[[3-(trifluoromethyl)phenyl]methylamino]pyrimidine-5-carbonitrile;
2-(dimethylamino)-4-(2-furyl)-6-[[3-(trifluoromethyl)phenyl]methylamino]pyrimidine-5-carbonitrile;
4-(2-furyl)-2-methoxy-6-[[3-(trifluoromethyl)phenyl]methylamino]pyrimidine-5-carbonitrile;
2-ethoxy-4-(2-furyl)-6-[[3-(trifluoromethyl)phenyl]methylamino]pyrimidine-5-carbonitrile;
2-butoxy-4-(2-furyl)-6-[[3-(trifluoromethyl)phenyl]methylamino]pyrimidine-5-carbonitrile;
4-[(6-bromo-2,3-dimethoxy-phenyl)methylamino]-6-(2-furyl)-2-methyl-pyrimidine-5-carbonitrile;
4-[(3-bromo-4-isopropoxy-phenyl)methylamino]-6-(2-furyl)-2-methyl-pyrimidine-5-carbonitrile;
4-(2-furyl)-2-methyl-6-[[1-(2-methylsulfonylphenyl)cyclopropyl]amino]pyrimidine-5-carbonitrile;
4-(benzylamino)-6-(2-furyl)-2-methyl-pyrimidine-5-carbonitrile;
4-[(2-chlorophenyl)methylamino]-6-(2-furyl)pyrimidine-5-carbonitrile;
4-(benzylamino)-6-(2-furyl)pyrimidine-5-carbonitrile;
4-(2-furyl)-6-(isobutylamino)pyrimidine-5-carbonitrile;
4-(cyclohexylmethylamino)-6-(2-furyl)pyrimidine-5-carbonitrile;
4-(2-furyl)-6-(2-phenylethylamino)pyrimidine-5-carbonitrile;
4-(cyclopentylmethylamino)-6-(2-furyl)pyrimidine-5-carbonitrile;
4-(2-furyl)-6-[(1-phenylcyclopentyl)methylamino]pyrimidine-5-carbonitrile;
4-[(2-aminophenyl)methylamino]-6-(2-furyl)pyrimidine-5-carbonitrile;
6-(2-furyl)-N2,N2-dimethyl-N4-[1-[3-(trifluoromethyl)phenyl]ethyl]pyrimidine-2,4-diamine;
N4-[(2-aminophenyl)methyl]-6-(2-furyl)-N2,N2-dimethyl-pyrimidine-2,4-diamine;
6-(2-furyl)-2-methoxy-N-[1-[3-(trifluoromethyl)phenyl]ethyl]pyrimidin-4-amine;
6-(2-furyl)-5-methoxy-N4-[[3-(trifluoromethyl)phenyl]methyl]pyrimidine-2,4-diamine;
N4-[(2-aminophenyl)methyl]-6-(2-thienyl)pyrimidine-2,4-diamine;
2-amino-4-[(4-chlorophenyl)methylamino]-6-(2-furyl)pyrimidine-5-carboxylic acid;
2-amino-4-(2-furyl)-6-[[3-(trifluoromethyl)phenyl]methylamino]pyrimidine-5-carboxylic acid;
2-amino-4-(2-furyl)-6-(o-tolylmethylamino)pyrimidine-5-carboxylic acid;
2-amino-4-(2-furyl)-6-[(2-methoxyphenyl)methylamino]pyrimidine-5-carboxylic acid;
2-amino-4-(cyclopentylmethylamino)-6-(2-furyl)pyrimidine-5-carboxylic acid;
2-amino-4-[benzyl(methyl)amino]-6-(2-furyl)pyrimidine-5-carboxylic acid;
4-(cyclopentylmethylamino)-2-(dimethylamino)-6-(2-furyl)pyrimidine-5-carboxylic acid;
ethyl 2-amino-4-[(6-bromo-2,3-dimethoxy-phenyl)methylamino]-6-(2-furyl)pyrimidine-5-carboxylate;
ethyl 4-(2-furyl)-2-methylsulfanyl-6-[[3-(trifluoromethyl)phenyl]methylamino]pyrimidine-5-carboxylate;
4-(2-furyl)-2-(2-methoxyethylamino)-6-[[3-(trifluoromethyl)phenyl]methylamino]pyrimidine-5-carboxylic acid;
ethyl 2-(2-azaspiro[3.3]heptan-2-yl)-4-(2-furyl)-6-[[3-(trifluoromethyl)phenyl]methylamino]pyrimidine-5-carboxylate;
2-(2-azaspiro[3.3]heptan-2-yl)-4-(2-furyl)-6-[[3-(trifluoromethyl)phenyl]methylamino]pyrimidine-5-carboxylic acid;
ethyl 4-(2-furyl)-2-(isobutylamino)-6-[[3-(trifluoromethyl)phenyl]methylamino]pyrimidine-5-carboxylate;
4-(2-furyl)-2-(isobutylamino)-6-[[3-(trifluoromethyl)phenyl]methylamino]pyrimidine-5-carboxylic acid;
4-(2-furyl)-2-(3-methoxypropylamino)-6-[[3-(trifluoromethyl)phenyl]methylamino]pyrimidine-5-carboxylic acid;
[2-amino-4-(2-furyl)-6-[[3-(trifluoromethyl)phenyl]methylamino]pyrimidin-5-yl]-(azetidin-1-yl)methanone;
[2-amino-4-(2-furyl)-6-[[3-(trifluoromethyl)phenyl]methylamino]pyrimidin-5-yl]-pyrrolidin-1-yl-methanone;
[2-amino-4-(2-furyl)-6-[[3-(trifluoromethyl)phenyl]methylamino]pyrimidin-5-yl]-(1-piperidyl)methanone;
[2-amino-4-(2-furyl)-6-[[3-(trifluoromethyl)phenyl]methylamino]pyrimidin-5-yl]-morpholino-methanone;
1 tert-butyl 4-[2-amino-4-(2-furyl)-6-[[3-(trifluoromethyl)phenyl]methylamino]pyrimidine-5-carbonyl]piperazine-1-carboxylate;
ethyl 4-[(2-chlorophenyl)methylamino]-6-(2-furyl)pyrimidine-5-carboxylate;
4-(2-furyl)-2-(2-methoxyethylamino)-6-[[1-[3-(trifluoromethyl)phenyl]cyclopropyl]amino]pyrimidine-5-carboxylic acid;
4-[(3-chlorophenyl)methylamino]-6-(2-furyl)-2-(2-methoxyethylamino)pyrimidine-5-carboxylic acid;
2-(ethylamino)-4-(2-furyl)-6-[[1-[3-(trifluoromethyl)phenyl]cyclopropyl]amino]pyrimidine-5-carboxylic acid;
ethyl 4-(2-furyl)-2-methyl-6-[[3-(trifluoromethyl)phenyl]methylamino]pyrimidine-5-carboxylate; and
ethyl 4-[(6-bromo-2,3-dimethoxy-phenyl)methylamino]-6-(2-furyl)-2-isopropyl-pyrimidine-5-carboxylate;
or a pharmaceutically acceptable salt thereof.

22. A compound according to any one of claims 1 to 20, selected from
2-amino-4-(2-furyl)-6-[(2,2,6,6-tetramethyltetrahydropyran-4-yl)methylamino]pyrimidine-5-carbonitrile;
2-amino-4-(2-furyl)-6-[1-[3-(trifluoromethyl)phenyl]ethylamino]pyrimidine-5-carbonitrile;
2-amino-4-(2-furyl)-6-[[1-(2-methylsulfonylphenyl)cyclopropyl]amino]pyrimidine-5-carbonitrile;
2-(ethylamino)-4-(2-furyl)-6-[[3-(trifluoromethyl)phenyl]methylamino]pyrimidine-5-carbonitrile;
2-butoxy-4-(2-furyl)-6-[[3-(trifluoromethyl)phenyl]methylamino]pyrimidine-5-carbonitrile;
4-(2-furyl)-2-methyl-6-[[1-(2-methylsulfonylphenyl)cyclopropyl]amino]pyrimidine-5-carbonitrile;
4-[(2-chlorophenyl)methylamino]-6-(2-furyl)pyrimidine-5-carbonitrile;
4-(benzylamino)-6-(2-furyl)pyrimidine-5-carbonitrile;
4-(cyclopentylmethylamino)-6-(2-furyl)pyrimidine-5-carbonitrile;
4-[(2-aminophenyl)methylamino]-6-(2-furyl)pyrimidine-5-carbonitrile;
2-amino-4-[(4-chlorophenyl)methylamino]-6-(2-furyl)pyrimidine-5-carboxylic acid;
2-amino-4-(2-furyl)-6-[[3-(trifluoromethyl)phenyl]methylamino]pyrimidine-5-carboxylic acid; and
4-[(3-chlorophenyl)methylamino]-6-(2-furyl)-2-(2-methoxyethylamino)pyrimidine-5-carboxylic acid;
or a pharmaceutically acceptable salt thereof.

23. A process for the preparation of a compound according to any one of claims 1 to 22 comprising at least one of the following steps:
(a) Substitution of a compound of formula (V), 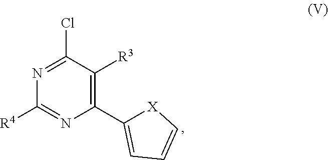
with a compound of formula (III), 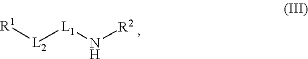
in the presence of a base;
(b) Coupling of a compound of formula (IV), 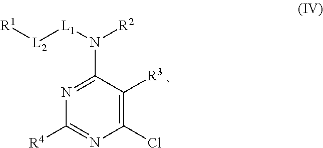
with a compound of formula (II), 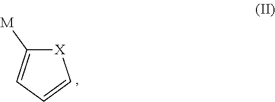
in the presence of Pd catalyst;
(c) Substitution of a compound of formula (XV), 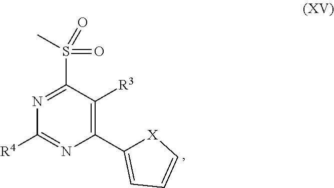
with a compound of formula (III), in the presence of a base;
(d) Substitution of a compound of formula (XII), 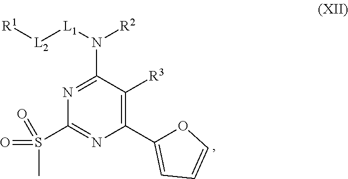
with a compound of formula (XII-1), R⁴H (XII-1);
(e) Hydrolysis of a compound of formula (I-3), 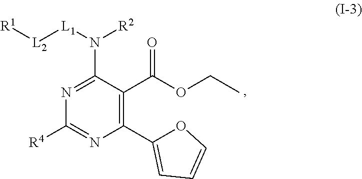
in the presence of a base;
(f) Coupling of a compound of formula (I-4), 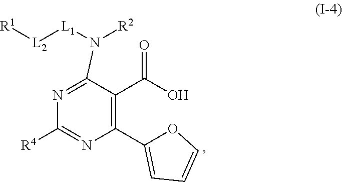
with a compound of formula (XVI), R⁵H (XVI), in the presence of a coupling reagent and a base;
wherein R¹ to R⁴, L₁, L₂ and X are defined as any one of claims 1 to 20; R⁵ is C₁₋₆ alkoxy, 4-6 membered heterocyclyl or C₁₋₆ alkoxycarbonyl(4-6 membered heterocyclyl); M is B(OH)₂ or SnBu₃.

24. A compound according to any one of claims 1 to 22 for use as therapeutically active substance.

25. A pharmaceutical composition comprising a compound in accordance with any one of claims 1 to 22 and a therapeutically inert carrier.

26. The use of a compound according to any one of claims 1 to 22 for the treatment or prophylaxis of HBV infection.

27. The use of a compound according to any one of claims 1 to 22 for the preparation of a medicament for the treatment or prophylaxis of HBV infection.

28. The use of a compound according to any one of claims 1 to 22 for the inhibition of cccDNA.

29. The use of a compound according to any one of claims 1 to 22 for the inhibition of HBeAg.

30. The use of a compound according to any one of claims 1 to 22 for the inhibition of HBsAg.

31. The use of a compound according to any one of claims 1 to 22 for the inhibition of HBV DNA.

32. A compound according to any one of claims 1 to 22 for use in the treatment or prophylaxis of HBV infection.

33. A compound according to any one of claims 1 to 22, when manufactured according to a process of claim 23.

34. A method for the treatment or prophylaxis of HBV infection, which method comprises administering an effective amount of a compound as defined in any one of claims 1 to 22.
